(19) United States
(12) Patent Application Publication
(10) Pub. No.: US 2022/0152176 A1
(43) Pub. Date: May 19, 2022

(54) CANCER BIOMARKERS FOR DURABLE CLINICAL BENEFIT

(71) Applicant: BIONTECH US INC., Cambridge, MA (US)

(72) Inventors: Lakshmi SRINIVASAN, Lexington, MA (US); Ying Sonia TING, Malden, MA (US); Meghan E. BUSHWAY, Somerville, MA (US); Kristen BALOGH, Somerville, MA (US); Julian SCHERER, Sherborn, MA (US)

(21) Appl. No.: 17/599,179

(22) PCT Filed: Mar. 27, 2020
(86) PCT No.: PCT/US2020/025497

§ 371 Date: Sep. 28, 2021

Related U.S. Application Data
(60) Provisional application No. 62/826,813, filed on Mar. 29, 2019.
(60) Provisional application No. 62/914,767, filed on Oct. 14, 2019.
(60) Provisional application No. 62/986,418, filed on Mar. 6, 2020.

(51) Int. Cl. A61K 39/00; A61K 45/06; A61P 17/00; A61P 35/00; C12Q 1/6886

(52) U.S. Cl. CPC A61K 39/0011; A61K 45/06; A61P 17/00; A61K 39/39558; C12Q 1/6886; A61K 2039/70; A61K 2039/876; A61P 35/00

(57) ABSTRACT

The present disclosure concerns methods of treating cancer with neoantigenic peptides such that durable clinical benefits are obtained, and compositions and methods for determining whether DCB can be predicted or assessed for a patient to be treated with a therapeutic comprising neoantigen.

### CROSS REFERENCE

This application claims the benefit of U.S. Provisional Application No. 62/826,813, filed on Mar. 29, 2019; U.S. Provisional Application No. 62/914,767, filed on Oct. 14, 2019; and U.S. Provisional Application No. 62/986,418, filed on Mar. 6, 2020, all of which are incorporated herein by reference in its entirety.

### BACKGROUND

The tumor microenvironment (TME) is complex and is considerably different from a comparable non-tumor tissue in both its physiology and architecture. On one hand the TME is conducive to tumor growth, but the anti-tumor agents are concentrated in the region as well. The latter includes various cell types, cytokines, chemokines, growth factors, cell-to-cell signaling agents, extracellular matrix components and soluble factors. Critical analysis of the pro-tumor and anti-tumor agents in this complex milieu of a tumor can provide useful TME signatures for accurately determining the state of a tumor and can be used to manipulate an on-treatment clinical procedure or direct a future clinical strategy. More importantly, TME signature can help determine clinical procedures towards a durable clinical benefit (DCB).

Precise evaluation of the immune response at the primary tumor site could be useful for understanding the development and monitoring of immune therapies for this disease.

### SUMMARY

The present disclosure provides, inter alia, a set of signatures or biomarkers associated with a tumor, a combination or subset of which may be used to determine the likelihood that a patient having the tumor would respond favorably to a treatment, such as treatment with a therapeutic agent comprising neoantigen peptides. In one aspect, the present disclosure provides one or more biomolecular signatures from a biological sample of a subject having or like to have a tumor, the one or more biological signatures are from a pre-treatment time-point with a therapeutic agent, a time-point during the treatment, and/or at the time after a certain treatment has been administered, and wherein the signature(s) relates to the subject's likelihood of responding to the treatment. In some embodiments, the therapeutic agent comprises (a) a one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein. Knowing and understanding the tumor and TME of a patient can directly affect clinical procedures. In some embodiment, a patient can be administered a first therapeutic agent comprising one or more neoantigen peptides and may be administered an altered dose of the first therapeutic agent, or administered the first therapeutic agent at an altered time interval of dosing, or may be administered a second therapeutic agent with or without the one or more neoantigenic peptides.

In one aspect, provided herein is a method of treating a patient having a tumor comprising: determining if a biological sample collected from the patient is positive or negative for a signature or biomarker which predicts that the patient is likely to have an anti-tumor response to a first therapeutic agent comprising (i) a one or more peptides comprising a neoepitope of a protein, (ii) a polynucleotide encoding the one or more peptides, (iii) one or more APCs comprising the one or more peptides or the polynucleotide encoding the one or more peptides, or (iv) a T cell receptor (TCR) specific for a neoepitope of the one or more peptides in complex with an HLA protein, and treating the patient with a therapeutic regimen that comprises the first therapeutic agent if the signature or biomarker is present; or treating the patient with a therapeutic regimen that does not include the first therapeutic agent if the signature or biomarker is absent, wherein the biomarker comprises at least a tumor microenvironment (TME) signature. In some embodiments, absence of a particular biomarker may be the signature for that biomarker, and the method of treating a patient, as described herein may include, for example, treating the patient with a therapeutic regimen that comprises the first therapeutic agent if the biomarker is absent; or treating the patient with a therapeutic regimen that does not include the first therapeutic agent if the biomarker is present.

In some embodiments, the signature or biomarker may include, inter alia, a tumor cell signature or biomarker, for example, determined in a biological sample excised from the tumor. In some embodiments, the signature or biomarker may include a signature or biomarker present in peripheral blood, for example, determined in a peripheral blood sample, or a biological sample collected from a distal or peripheral tissue, cell or body fluid.

In some embodiments, the TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, an MEW class II signature or a functional Ig CDR3 signature.

In some embodiments, the B-cell signature comprises expression of a gene comprising CD20, CD21, CD3, CD22, CD24, CD27, CD38, CD40, CD72, CD79a, IGKC, IGHD, MZB1, MS4A1, CD138, BLK, CD19, FAM30A, FCRL2, MS4A1, PNOC, SPIB, TCL1A, TNFRSF17 or combinations thereof.

In some embodiments, the TLS signature indicates formation of tertiary lymphoid structures. In some embodiments, the tertiary lymphoid structure represents aggregates of lymphoid cells.

In some embodiments, the TLS signature comprises expression of a gene comprising CCL18, CCL19, CCL21, CXCL13, LAMP3, LTB, MS4A1, or combinations thereof.

In some embodiments, the TIS signature comprises an inflammatory gene, a cytokine, a chemokine, a growth factor, a cell surface interaction protein, a granulation factor, or a combination thereof.

In some embodiments, the TIS signature comprises CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT or a combination thereof.

In some embodiments, the effector/memory-like CD8+ T cell signature comprises expression of a gene comprising CCR7, CD27, CD45RO, CCR7, FLT3LG, GRAP2, IL16, IL7R, LTB, S1PR1, SELL, TCF7, CD62L, or any combination thereof.

In some embodiments, the HLA-E/CD94 signature comprises expression of a gene CD94 (KLRD1), CD94 ligand, HLA-E, KLRC1 (NKG2A), KLRB1 (NKG2C) or any combination thereof.

In some embodiments, the HLA-E/CD94 signature further comprises an HLA-E:CD94 interaction level.

In some embodiments, the NK cell signature comprises expression of a gene CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2 or a combination thereof.

In some embodiments, the MHC class II signature comprises expression of a gene that is an HLA comprising HLA-DMA, HLA-DOA, HLA-DPA1, HLA-DPB1, HLA-DQB1, HLA-DRA, HLA-DRB1, HLA-DRB5 or a combination thereof.

In some embodiments, the biomarker comprises a subset of TME gene signature comprising a Tertiary Lymphoid Structures (TLS) signature; wherein the TLS signature comprises a gene CCL18, CCL19, CCL21, CXCL13, LAMP3, LTB, MS4A1, or combinations thereof.

In some embodiments, the functional Ig CDR3 signature comprises an abundance of functional Ig CDR3s.

In some embodiments, the abundance of functional Ig CDR3s is determined by RNA-seq. In some embodiments, the abundance of functional Ig CDR3s is an abundance of functional Ig CDR3s from cells of a TME sample from a subject. In some embodiments, the abundance of functional Ig CDR3s is 2\hat{ }7 or more functional Ig CDR3s.

In some embodiments, the method further comprises: administering to the biomarker positive patient the first therapeutic agent, an altered dose or time interval of the first therapeutic agent, or a second therapeutic agent.

In some embodiments, the method further comprises: not administering to the biomarker negative patient the first therapeutic agent or a second therapeutic agent.

In some embodiments, the method further comprises administering to the biomarker positive patient, an increased dose of the first therapeutic agent.

In some embodiments, the method further comprises modifying a time interval of administration of the first therapeutic agent to the biomarker positive or negative patient.

In one aspect, provided herein is a method for testing a patient having a tumor for the presence or absence of a baseline biomarker that predicts that the patient is likely to have an anti-tumor response to a treatment with a therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, the method comprising: obtaining a baseline sample that has been isolated from the tumor of the patient; measuring the baseline expression level of each gene in a tumor microenvironment (TME) gene or a subset of said genes; normalizing the measured baseline expression levels; calculating a baseline signature score for the TME gene signature from the normalized expression levels; comparing the baseline signature score to a reference score for the TME gene signature; and, classifying the patient as biomarker positive or biomarker negative for an outcome related to a durable clinical benefit (DCB) from the therapeutic agent.

In some embodiments, the TME signature comprises a signature described herein or a subset thereof.

In one aspect, provided herein is a pharmaceutical composition for use in treating cancer in a patient who tests positive for a biomarker, wherein the composition the therapeutic agent comprises (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein; and at least one pharmaceutically acceptable excipient; and wherein the biomarker is an on-treatment biomarker which comprises a gene signature selected from the group consisting of TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature. In some embodiments, a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature provides a signature for a predictive durable clinical benefit (DCB) for the treatment.

In some embodiments, the TME signature comprises a signature described herein or a subset thereof.

In one aspect, provided herein is a method of treating cancer in a subject in need thereof, comprising: administering a therapeutically effective amount of a cancer therapeutic agent, wherein the subject has an increased likelihood of responding to the cancer therapeutic agent, wherein the subject's increased likelihood of responding to the cancer therapeutic agent is associated with the presence of one or more peripheral blood mononuclear cell signatures prior to treatment with the cancer therapeutic agent; and wherein at least one of the one or more peripheral blood mononuclear cell signatures comprises a threshold value for a ratio of cell counts of a first mononuclear cell type to a second mononuclear cell type in the peripheral blood of the subject.

In some embodiments, the cancer is melanoma.

In some embodiments, the cancer is non-small cell lung cancer.

In some embodiments, the cancer is bladder cancer.

In some embodiments, the cancer therapeutic comprises a neoantigen peptide vaccine.

In some embodiments, the cancer therapeutic comprises an anti-PD1 antibody.

In some embodiments, the cancer therapeutic comprises a combination of the neoantigen vaccine and the anti-PD1 antibody.

In some embodiments, the anti-PD1 antibody is nivolumab.

In some embodiments, the threshold value is a maximum threshold value.

In some embodiments, the threshold value is a minimum threshold value.

In some embodiments, at least one of the one or more peripheral blood mononuclear cell signatures comprises a maximum threshold value for a ratio of naïve CD8+ T cells to total CD8+ T cells in a peripheral blood sample from the subject.

In some embodiments, the maximum threshold value for the ratio of naïve CD8+ T cells to total CD8+ T cells in the peripheral blood sample from the subject is about 20:100.

In some embodiments, the peripheral blood sample from the subject has a ratio of naïve CD8+ T cells to total CD8+ T cells that is 20:100 or less or less than 20:100.

In some embodiments, at least one of the one or more peripheral blood mononuclear cell signatures comprises a minimum threshold value for a ratio of effector memory CD8+ T cells to total CD8+ T cells in a peripheral blood sample from the subject.

In some embodiments, the minimum threshold value for the ratio of effector memory CD8+ T cells to total CD8+ T cells in the peripheral blood sample from the subject is about 40:100.

In some embodiments, the peripheral blood sample from the subject has a ratio of effector memory CD8+ T cells to total CD8+ T cells that is 40:100 or more or more than 40:100.

In some embodiments, at least one of the one or more peripheral blood mononuclear cell signatures comprises a minimum threshold value for a ratio of class-switched memory B cells to total CD19+ B cells in a peripheral blood sample from the subject.

In some embodiments, the minimum threshold value for the ratio of class-switched memory B cells to total CD19+ B cells in the peripheral blood sample from the subject is about 10:100.

In some embodiments, the peripheral blood sample from the subject has a ratio of class-switched memory B cells to total CD19+ B cells that is 10:100 or more or more than 10:100.

In some embodiments, at least one of the one or more peripheral blood mononuclear cell signatures comprises a maximum threshold value for a ratio of naïve B cells to total CD19+ B cells in a peripheral blood sample from the subject.

In some embodiments, the maximum threshold value for the ratio of naïve B cells to total CD19+ B cells in the peripheral blood sample from the subject is about 70:100.

In some embodiments, the peripheral blood sample from the subject has a ratio of naïve B cells to total CD19+ B cells that is 70:100 or less or less than 70:100.

In some embodiments, the cancer is a melanoma.

In some embodiments, at least one of the one or more peripheral blood mononuclear cell signatures comprises a maximum threshold value for a ratio of plasmacytoid dendritic cells to total Lin−/CD11c− cells in a peripheral blood sample from the subject.

In some embodiments, the maximum threshold value for the ratio of plasmacytoid dendritic cells to total Lin−/CD11c− cells in the peripheral blood sample from the subject is about 3:100.

In some embodiments, the peripheral blood sample from the subject has a ratio of plasmacytoid dendritic cells to total Lin−/CD11c− cells that is 3:100 or less or less than 3:100.

In some embodiments, at least one of the one or more peripheral blood mononuclear cell signatures comprises a maximum threshold value for a ratio of CTLA4+ CD4 T cells to total CD4+ T cells in a peripheral blood sample from the subject

In some embodiments, the maximum threshold value for the ratio of CTLA4+ CD4 T cells to total CD4+ T cells in the peripheral blood sample from the subject is about 9:100.

In some embodiments, the peripheral blood sample from the subject has a ratio of CTLA4+ CD4 T cells to total CD4+ T cells that is 9:100 or less or less than 9:100.

In some embodiments, the cancer is a non-small cell lung cancer.

In some embodiments, at least one of the one or more peripheral blood mononuclear cell signatures comprises a minimum threshold value for a ratio of memory CD8+ T cells to total CD8+ T cells in a peripheral blood sample from the subject.

In some embodiments, the minimum threshold value for the ratio of memory CD8+ T cells to total CD8+ T cells in the peripheral blood sample from the subject is about 40:100.

In some embodiments, the peripheral blood sample from the subject has a ratio of memory CD8+ T cells to total CD8+ T cells that is 40:100 or more or more than 40:100. In some embodiments, the peripheral blood sample from the subject has a ratio of memory CD8+ T cells to total CD8+ T cells that is 55:100 or more or more than 55:100.

In some embodiments, the cancer is a bladder cancer.

Also provided herein is a method of treating cancer in a subject in need thereof, comprising: administering to the subject a therapeutically effective amount of a cancer therapeutic agent, wherein the subject has an increased likelihood of responding to the cancer therapeutic agent, and wherein the subject's increased likelihood of responding to the cancer therapeutic agent is associated with a clonal composition characteristic of TCR repertoires analyzed from peripheral blood sample of the subject at least at a timepoint prior to administering the cancer therapeutic agent. In some embodiments, the clonal composition characteristic of the TCR repertoires provides a signature for a predictive durable clinical benefit (DCB) for the treatment.

In some embodiments, the clonal composition characteristic of TCR repertoires in a prospective patient is defined by a relatively low TCR diversity versus the TCR diversity in healthy donors.

In some embodiments, the clonal composition characteristic is analyzed by a method comprising sequencing the TCRs or fragments thereof.

In some embodiments, the clonal composition characteristic of TCR repertoires is defined by the clonal frequency distribution of the TCRs.

In some embodiments, the clonal composition characteristic of the TCR repertoires is further analyzed by calculating the frequency distribution pattern of TCR clones.

In some embodiments, the frequency distribution pattern of TCR clones is analyzed using one or more of: Gini Coefficient, Shannon entropy, DE50, Sum of Squares, and Lorenz curve.

In some embodiments, the subject's increased likelihood of responding to the cancer therapeutic agent is associated with increased clonality of the TCRs.

In some embodiments, the subject's increased likelihood of responding to the cancer therapeutic agent is associated with increased frequency of medium and/or large and/or hyperexpanded sized TCR clones.

In some embodiments, the subject's increased likelihood of responding to the cancer therapeutic agent is associated with a clonal composition characteristic of TCR repertoires according to any one of embodiments described, wherein the clonal composition characteristic is analyzed from peripheral blood sample of the subject prior to administering a therapeutically effective amount of a cancer therapeutic agent.

In some embodiments, a clonal composition characteristic of TCR repertoires comprises a measure of the clonal stability of the TCRs.

In some embodiments, the clonal stability of the TCRs is analyzed as TCR turnover between a first and a second timepoints, wherein the first timepoint is prior to administering the cancer therapeutic agent and the second timepoint is a timepoint during the duration of the treatment.

In some embodiments, the second timepoint is prior to administering the vaccine.

In some embodiments, the clonal stability of TCRs is analyzed using a Jensen-Shannon Divergence.

In some embodiments, the subject's increased likelihood of responding to the cancer therapeutic agent is associated with higher TCR stability.

In some embodiments, the subject's increased likelihood of responding to the cancer therapeutic agent is associated with reduced turnover of T cell clones between the first timepoint and the second timepoint.

In some embodiments, the clonal composition characteristic is analyzed from peripheral blood sample of the subject prior to administering a vaccine, wherein the vaccine comprises at least one peptide or a polynucleotide encoding a peptide, wherein the cancer therapeutic agent comprises a combination of a neoantigen vaccine and an anti-PD1 antibody, wherein the neoantigen vaccine is administered or co-administered after a period of administering anti-PD1 antibody alone.

In one aspect, provided herein is a method of treating cancer in a subject in need thereof, comprising: administering a therapeutically effective amount of a cancer therapeutic agent to the subject, wherein the subject has an increased likelihood of responding to the cancer therapeutic agent, wherein the subject's increased likelihood of responding to the cancer therapeutic agent is associated with the presence of one or more genetic variations in the subject, wherein the subject has been tested for a presence of the one or more genetic variations with an assay and has been identified as having the one or more genetic variations, wherein the one or more genetic variations comprise an ApoE allele genetic variation comprising (i) an ApoE2 allele genetic variation comprising a sequence encoding a R158C ApoE protein or (ii) an ApoE4 allele genetic variation comprising a sequence encoding a C112R ApoE protein.

In some embodiments, the cancer therapeutic agent comprises a neoantigen peptide vaccine. In some embodiments, the cancer therapeutic agent further comprises an anti-PD1 antibody. In some embodiments, the cancer therapeutic agent does not comprise an anti-PD1 antibody monotherapy.

In some embodiments, the cancer is melanoma.

In some embodiments, the subject is homozygous for the ApoE2 allele genetic variation. In some embodiments, the subject is heterozygous for the ApoE2 allele genetic variation. In some embodiments, the subject is homozygous for the ApoE4 allele genetic variation. In some embodiments, the subject is heterozygous for the ApoE4 allele genetic variation. In some embodiments, the subject comprises an ApoE allele comprising a sequence encoding a ApoE protein that is not a R158C ApoE protein or a C112R ApoE protein. In some embodiments, the subject comprises an ApoE3 allele comprising a sequence encoding a ApoE protein that is not a R158C ApoE protein or a C112R ApoE protein.

In some embodiments, the subject has rs7412-T and rs449358-T.

In some embodiments, the subject has rs7412-C and rs449358-C.

In some embodiments, a reference subject that is homozygous for the ApoE3 allele has a decreased likelihood of responding to the cancer therapeutic agent.

In some embodiments, the assay is a genetic assay.

In some embodiments, the cancer therapeutic agent comprises one or more peptides comprising a cancer epitope.

In some embodiments, the cancer therapeutic agent comprises a polynucleotide encoding one or more peptides comprising a cancer epitope, or, (ii) one or more APCs comprising the one or more peptides or the polynucleotide encoding the one or more peptides, or (iii) a T cell receptor (TCR) specific for a cancer epitope of the one or more peptides in complex with an HLA protein.

In some embodiments, the cancer therapeutic agent further comprises an immunomodulatory agent.

In some embodiments, the immunotherapeutic agent is an anti-PD1 antibody.

In some embodiments, the cancer therapeutic agent is not nivolumab alone or pembrolizumab alone.

In some embodiments, the one or more genetic variations comprises chr19:44908684 T>C; wherein chromosome positions of the one or more genetic variations are defined with respect to UCSC hg38.

In some embodiments, the one or more genetic variations comprises chr19:44908822 C>T; wherein chromosome positions of the one or more genetic variations are defined with respect to UCSC hg38.

In some embodiments, the method further comprises testing the subject for the presence of the one or more genetic variations with the assay prior to the administering.

In some embodiments, the ApoE2 allele genetic variation is a germline variation.

In some embodiments, the ApoE4 allele genetic variation is a germline variation.

In one aspect, provided herein is a method treating a cancer in a subject, comprising: administering to the subject a cancer therapeutic agent comprising one or more peptides comprising a cancer epitope; wherein the subject is determined as having the germline ApoE4 allelic variant.

In some embodiments, the therapeutic agent further comprises one or more of: an adjuvant therapy, a cytokine therapy, or an immunomodulator therapy.

In some embodiments, the immunomodulator therapy is a PD1 inhibitor, such as an anti-PD1 antibody. In some embodiments, the therapeutic agent does not comprise a PD1 inhibitor monotherapy.

In some embodiments, the method further comprises administering an agent that promotes ApoE activity or comprises ApoE activity. In some embodiments, the method further comprises administering an agent that promotes ApoE-like activity or comprises ApoE-like activity. In some embodiments, a subject that is homozygous for the ApoE4 allele has an increased likelihood of responding to the cancer therapeutic agent. In some embodiments, the method further comprises administering an agent that promotes ApoE4 activity or comprises ApoE4 activity. In some embodiments, the method further comprises administering an agent that promotes ApoE4-like activity or comprises ApoE4-like activity. In some embodiments, a reference subject having reduced NMDA or AMPA receptor functions may have an increased likelihood of responding to the cancer therapeutic agent. For example, the method can further comprise administering an agent that reduces NMDA or AMPA receptor functions. In some embodiments, a subject having higher intracellular calcium levels in neuronal cells may have an increased likelihood of responding to the cancer therapeutic agent. In some embodiments, the method can further comprise administering an agent that increases intracellular calcium levels in neuronal cells. In some embodiments, the method can further comprise administering an agent that alters calcium response to NMDA in neuronal cells. In some embodiments, a subject having impaired glutamatergic neurotransmission may have an increased likelihood of responding to the cancer therapeutic agent. In some embodiments, the method can further comprise administering an agent that impairs glutamatergic neurotransmission. In some embodiments, a subject having an enhanced Aβ oligomerization may have an increased likelihood of responding to the cancer therapeutic agent. In some embodiments, a subject having a predisposition to Alzheimer's disease may have an increased likelihood of responding to the cancer therapeutic agent. In some embodiments, a subject having increased serum vitamin D levels may have an increased likelihood of responding to the cancer therapeutic agent. In some embodiments, the method can further comprise administering an agent that increases serum vitamin D levels. In some embodiments, a subject having cells with low cholesterol efflux may have an increased likelihood of responding to the cancer therapeutic agent. In some embodiments, the method can further comprise administering an agent that lowers cholesterol efflux from cells of the subject. In some embodiments, a subject having high total cholesterol (TC) levels (e.g., higher total cholesterol (TC) levels than a subject having ApoE3 homozygous genotype) may have an increased likelihood of responding to the cancer therapeutic agent. In some embodiments, the method can further comprise administering an agent that increases TC levels. In some embodiments, a subject having high LDL levels (e.g., higher LDL levels than a subject having ApoE3 homozygous genotype) may have an increased likelihood of responding to the cancer therapeutic agent. In some embodiments, the method can further comprise administering an agent that increases LDL levels. In some embodiments, a subject having low HDL levels (e.g., lower HDL levels than a subject having ApoE3 homozygous genotype) may have an increased likelihood of responding to the cancer therapeutic agent. In some embodiments, the method can further comprise administering an agent that decreases HDL levels. In some embodiments, a reference subject may have an lower TC, and/or a lower LDL and/or a higher HDL level compared to a subject having ApoE3 homozygous genotype, and may have a decreased likelihood of responding to the cancer therapeutic agent. In some embodiments, a reference subject may have a higher TC, and/or a higher LDL and/or a lower HDL level compared to a subject having ApoE3 homozygous genotype, and may have an increased likelihood of responding to the cancer therapeutic agent. In some embodiments, a subject having low APOE levels in the cerebrospinal fluid (CSF) plasma or interstitial fluid (e.g., lower APOE levels in the cerebrospinal fluid (CSF) plasma or interstitial fluid) than a subject having ApoE3 homozygous genotype) may have an increased likelihood of responding to the cancer therapeutic agent. In some embodiments, the method can further comprise administering an agent that decreases APOE levels in the CSF, plasma or interstitial fluid.

In some embodiments, the method further comprises administering an agent that inhibits ApoE activity. In some embodiments, the method further comprises administering an agent that inhibits ApoE4 activity. In some embodiments, the method further comprises administering an agent that inhibits ApoE2 activity. In some embodiments, the method further comprises administering an agent that inhibits ApoE3 activity.

In one aspect, provided herein is a method of treating a patient having a tumor comprising: determining if a sample collected from the patient is positive or negative for a biomarker which predicts that the patient is likely to have an anti-tumor response to a first therapeutic agent comprising (i) a one or more peptides comprising a neoepitope of a protein, (ii) a polynucleotide encoding the one or more peptides, (iii) one or more APCs comprising the one or more peptides or the polynucleotide encoding the one or more peptides, or (iv) a T cell receptor (TCR) specific for a neoepitope of the one or more peptides in complex with an HLA protein, and (b) treating the patient with a therapeutic regimen that comprises the first therapeutic agent if the biomarker is present; or, treating the patient with a therapeutic regimen that does not include the first therapeutic agent if the biomarker is absent, wherein the biomarker comprises a TME signature.

In some embodiments, the TME signature comprises the TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, or an MHC class II signature.

In some embodiments, the B-cell signature comprises expression of a gene from the genes comprising: CD19, CD21, CD22, CD24, CD27, CD38, CD40, CD72, CD3, CD79a, CD79b, IGKC, IGHD, MZB1, TNFRSF17, MS4A1 (cd20), CD138, TNFRSR13B, GUSPB11, BAFFR, AID, IGHM, IGHE, IGHA1, IGHA2, IGHA3, IGHA4, BCL6, FCRLA or combinations thereof.

In some embodiments, the TLS signature comprises expression of a gene from the genes comprising CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, or combinations thereof.

In some embodiments, the TIS signature comprises CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT or a combination thereof.

In some embodiments, the effector/memory-like CD8+ T cell signature comprises expression of a gene from the genes or gene encoding comprising: CCR7, CD27, CD45RO, FLT3LG, GRAP2, IL16, IL7R, LTB, S1PR1, SELL, TCF7, CD62L, PLAC8, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PERI, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, or any combination thereof.

In some embodiments, the HLA-E/CD94 signature comprises expression of a gene from the genes CD94 (KLRD1), CD94 ligand, HLA-E, KLRC1 (NKG2A), KLRB1 (NKG2C) or any combination thereof.

In some embodiments, the HLA-E/CD94 signature further comprises an HLA-E:CD94 interaction level.

In some embodiments, the NK cell signature comprises expression of a gene from the genes CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, NCAM1, or a combination thereof.

In some embodiments, the MHC class II signature comprises expression of a gene from the genes that is an HLA comprising HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5 or a combination thereof.

In one embodiment, the method contemplated herein comprises (i) determining if a sample collected from the patient is positive or negative for a biomarker which predicts that the patient is likely to have an anti-tumor response to a first therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, and (ii) treating the patient with a therapeutic regimen that comprises the first therapeutic agent if the biomarker is present or treating the patient with a therapeutic regimen that does not include the first therapeutic agent if the biomarker is absent; wherein the biomarker comprises a subset of TME gene signature comprising a Tertiary Lymphoid Structures (TLS) signature; wherein the TLS signature comprises a genes from the genes CCL18, CCL19, CCL21, CXCL13, LAMP3, LTB, MS4A1, or combinations thereof.

In one aspect, provided herein is a method of treating cancer in a subject in need thereof, comprising: administering a therapeutically effective amount of a cancer therapeutic agent to the subject, wherein the subject has an increased likelihood of responding to the cancer therapeutic agent, wherein the subject's increased likelihood of responding to the cancer therapeutic agent is associated with the presence of one or more genetic variations in the subject, wherein the subject has been tested for a presence of the one or more genetic variations with an assay and has been identified as having the one or more genetic variations, wherein the one or more genetic variations comprise an ApoE allele genetic variation comprising (i) an ApoE2 allele genetic variation comprising a sequence encoding a R158C ApoE protein or (ii) an ApoE4 allele genetic variation comprising a sequence encoding a C112R ApoE protein. In some embodiments, the cancer is melanoma.

In some embodiments, the subject is homozygous for the ApoE2 allele genetic variation. In some embodiments, the subject is heterozygous for the ApoE2 allele genetic variation. In some embodiments, the subject is homozygous for the ApoE4 allele genetic variation. In some embodiments, the subject is heterozygous for the ApoE4 allele genetic variation. In some embodiments, the subject comprises an ApoE allele comprising a sequence encoding a ApoE protein that is not a R158C ApoE protein or a C112R ApoE protein. In some embodiments, the subject comprises an ApoE3 allele comprising a sequence encoding a ApoE protein that is not a R158C ApoE protein or a C112R ApoE protein. In some embodiments, the subject has rs7412-T and rs429358-T. In some embodiments, the subject has rs7412-C and rs429358-C. In some embodiments, a reference subject that is homozygous for the ApoE3 allele has a decreased likelihood of responding to the cancer therapeutic agent

In some embodiments, the assay is a genetic assay.

In some embodiments, the cancer therapeutic agent comprises (i) one or more peptides comprising a cancer epitope of a protein, (ii) a polynucleotide encoding the one or more peptides, (iii) one or more APCs comprising the one or more peptides or the polynucleotide encoding the one or more peptides, or (iv) a T cell receptor (TCR) specific for a cancer epitope of the one or more peptides in complex with an HLA protein.

In some embodiments, the cancer therapeutic agent comprises an immunosuppressive agent.

In some embodiments, the cancer therapeutic agent comprises an anti-PD1 antibody.

In some embodiments, the cancer therapeutic agent comprises nivolumab or pembrolizumab.

In some embodiments, the one or more genetic variations comprises chr19:44908684 T>C; wherein chromosome positions of the one or more genetic variations are defined with respect to UCSC hg38.

In some embodiments, the one or more genetic variations comprises chr19:44908822 C>T; wherein chromosome positions of the one or more genetic variations are defined with respect to UCSC hg38.

In some embodiments, the method further comprises testing the subject for the presence of the one or more genetic variations with the assay prior to the administering.

In some embodiments, the method further comprises administering to the biomarker positive patient the first therapeutic agent, an altered dose or time interval of the first therapeutic agent, or a second therapeutic agent.

In some embodiments, the method further comprises not administering to the biomarker positive patient the first therapeutic agent, an altered dose or time interval of the first therapeutic agent, or a second therapeutic agent.

In some embodiments, the method further comprises administering to the biomarker positive patient, an increased dose of the first therapeutic agent.

In some embodiments, the method further comprises modifying a time interval of administration of the first therapeutic agent to the biomarker positive or negative patient.

In one aspect, provided herein is a method testing a patient having a cancer or a tumor for the presence or absence of an on-treatment biomarker that predicts that the patient is likely to have an anti-tumor response to administering a first therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, the method comprising: (i) obtaining a representative baseline sample from a tumor collected from the patient; (ii) measuring in the baseline sample a baseline expression level of each gene in a TME signature; (iii) normalizing the measured baseline expression levels; (iv) calculating a baseline TME gene signature score for the TME gene signature from the normalized baseline expression levels; (v) obtaining a representative sample from the tumor that has been collected from the patient at a time post-treatment; (vi) measuring the post-treatment expression level of each gene in the TME gene signature in representative sample from the tumor that has been collected from the patient at a time period post-treatment; (vii) normalizing each of the measured post-treatment expression levels; (viii) calculating a post-treatment TME gene signature score for each gene in the TME gene signature from the normalized expression levels; (ix) calculating a post-treatment TME gene signature score for each gene in the TME gene signature from the measured expression levels; (x) comparing the post-treatment TME gene signature score to the baseline TME gene signature score, and (xi) classifying the patient as biomarker positive or biomarker negative for an outcome related to durable clinical benefit (DCB) from the first therapeutic agent; wherein obtaining, measuring, normalizing and calculating the baseline TME gene signature score can be performed before or concurrently with obtaining, measuring, normalizing and calculating the post-treatment TME gene signature score; and wherein a biomarker positive patient is determined to be likely experience a DCB with the first therapeutic agent.

In some embodiments, higher normalized expression of a gene compared to a normalized baseline expression in the TME gene signature is associated with a positive biomarker classification for DCB with the therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein.

In some embodiments, a patient with DCB has a higher normalized gene expression in B cell activation signature compared to a normalized baseline expression.

In some embodiments, a patient with DCB has a higher normalized gene expression in MHC class II signature compared to a normalized baseline expression.

In some embodiments, a patient with DCB has a higher normalized gene expression in NK cell signature compared to a normalized baseline expression.

In some embodiments, a patient with DCB has a higher normalized gene expression of CD94, and/or of HLA-E compared to a normalized baseline expression; and/or a higher HLA-E interaction with CD94.

In some embodiments, the method comprises a higher normalized gene expression of any one or more of genes or genes encoding CD19, CD20, CD21, CD3, CD22, CD24, CD27, CD38, CD40, CD72, CD79a, IGKC, IGHD, MZB1, TNFRSF17, MS4A1, CD138, CCL18, CCL19, CCL21, CXCL13, LAMP3, LTB, MS4A1, CCR7, CD27, CD45RO, FLT3LG, GRAP2, IL16, IL7R, LTB, S1PR1, SELL, TCF7, CD62L, CD94 (KLRD1), KLRC1 (NKG2A), KLRB1 (NKG2C), HLA-E, HLA-DMA, HLA-DOA, HLA-DPA1, HLA-DPB1, HLA-DQB1, HLA-DRA, CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, CCL5, CD27, CD274, CD276, CD8A, CMKLR1, CXCL9, CXCR6, HLA-DQA1, HLA-DRB1, HLA-DRB5, HLA-E, IDO1, LAG3, NKG7, PDCD1LG2, PSMB10, STAT1, TIGIT compared to a normalized baseline expression is associated with a positive biomarker classification for DCB with the therapeutic agent.

In some embodiments, a lower normalized expression of a gene compared to a normalized baseline expression in the TME gene signature is associated with a positive biomarker classification for DCB with the therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein.

In some embodiments, a lower normalized expression of B7-H3 is associated with a positive biomarker classification for DCB with the therapeutic agent.

In some embodiments, the increase in normalized expression of a gene compared to a normalized baseline expression ranges from about 1.1 to about 100 fold.

In some embodiments, the decrease in normalized expression of a gene compared to a normalized baseline expression ranges from about 1.1 to 100 fold.

In some embodiments, the cancer or the tumor is a melanoma.

In some embodiments, the gene signature from a tumor, a tumor microenvironment, or peripheral blood comprises a set of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or about 50 genes or gene products. In some embodiments, determination of durable clinical benefit of a treatment on a subject requires determination of gene signatures from a tumor, a tumor microenvironment, and/or peripheral blood comprising a set of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or about 50 genes or gene products.

In some embodiments, the therapeutic agent comprises one or more peptides comprising a neoepitope of a protein are selected from a group of peptides predicted by a HLA binding predictive platform, neonmhc (RECON) version 1, 2, or 3, wherein the HLA binding predictive platform is a computer based program with a machine learning algorithm, and where in the machine learning algorithm integrates a multitude of information related to a peptide and a human leukocyte antigen to which it associates, comprising peptide amino acid sequence information, structural information, association and or dissociation kinetics information and mass spectrometry information.

The method of any one of the preceding embodiments, wherein the one or more peptides comprising a neoepitope of a protein are shared neoantigens.

In some embodiments, the one or more peptides comprising a neoepitope of a protein are patient-specific neoantigens.

In some embodiments, the one or more peptides comprising a neoepitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or about 50 peptides. In some embodiments, the one or more peptides comprising a neoepitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or about 50 peptides encoded by multiple genes.

In some embodiments, the representative biological sample from the tumor comprises a tumor biopsy sample.

In some embodiments, the representative sample from the tumor comprises total RNA extracted from a cell, tissue, or fluid in a tumor.

In some embodiments, detecting within the representative sample from the TME gene signature of DCB is by real time quantitative PCR.

In some embodiments, detecting within the representative sample from the TME gene signature of DCB is by flow cytometry.

In some embodiments, detecting within the representative sample from the TME signature of DCB is by microarray analysis.

In some embodiments, detecting within the representative sample from the TME gene signature of DCB is by nanostring assay.

In some embodiments, detecting within the representative sample from the TME gene signature of DCB is by RNA sequencing.

In some embodiments, detecting within the representative sample from the TME gene signature of DCB is by single cell RNA sequencing.

In some embodiments, detecting within the representative sample from the TME gene signature of DCB is by ELISA.

In some embodiments, detecting within the representative sample from the TME gene signature of DCB is by ELISPOT.

In some embodiments, detecting within the representative sample from the TME gene signature of DCB is by mass spectrometry.

In some embodiments, detecting within the representative sample from the TME gene signature of DCB is by confocal microscopy.

In some embodiments, detecting within the representative sample from the TME gene signature of DCB is cellular cytotoxicity assay.

In some embodiments, co-administering to the patient one or more additional anti-tumor therapy.

In some embodiments, the obtaining the representative sample from the tumor comprises obtaining from an apheresis sample of the patient.

In some embodiments, the obtaining the representative sample from the tumor comprises obtaining a tumor biopsy sample.

In some embodiments, the obtaining a representative sample from the tumor comprises obtaining blood from the patient.

In some embodiments, the obtaining a representative sample from the tumor comprises obtaining a tissue fluid from the patient.

In some embodiments, the representative biological sample of the patient is isolated on day 0, or at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 1 month, 2 months, 3 months, 4 months, 5 months, 6 months, 1 year or at least 2 years after administering the therapeutic, wherein the therapeutic is the first therapeutic.

In some embodiments, comparing the post-treatment TME gene signature score to the baseline TME gene signature score comprises comparing a weighted average of TME gene signature score of a set of genes.

In some embodiments, the set of genes comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or about 50 genes.

In one aspect, provided herein is a method for determining induction of tumor neoantigen specific T cells in a tumor, the method comprising: detecting one or more tumor microenvironment (TME) signatures of durable clinical benefit (DCB) comprising: a B-cell signature, a Tertiary Lymphoid Structures (TLS) signature, an effector/memory-like CD8+ T cell signature, a HLA-E/CD94 interaction signature, a NK cell signature, and an MHC class II signature, wherein at least one of the signatures is altered compared to a corresponding representative sample before administering the composition.

In some embodiments, the one or more tumor microenvironment (TME) gene signatures of durable clinical benefit (DCB) further comprises a higher gene expression of CD107a, IFN-γ, or TNF-α, GZMA, GZMB, PRF1 compared to baseline measurements.

In some embodiments, the therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein comprises a neoantigen peptide vaccine.

In some embodiments, the representative baseline sample is the sample that has been collected from the patient at a time prior to treatment.

In some embodiments, the treatment comprises administration of the therapeutic agent comprising: (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein.

In some embodiments, the representative baseline sample is an archived sample.

In some embodiments, the representative baseline sample is archived sample from the patient.

In one aspect, provided herein is a pharmaceutical composition for use in treating cancer in a patient who tests positive for a biomarker, wherein the composition the therapeutic agent comprises (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein; and at least one pharmaceutically acceptable excipient; and wherein the biomarker is an on-treatment biomarker which comprises a gene signature selected from the group consisting of TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, the therapeutic agent is a neoantigen peptide vaccine.

In some embodiments, the TME gene signature comprises: a B-cell signature that comprises a gene comprising CD19, CD20, CD21, CD3, CD22, CD24, CD27, CD38, CD40, CD72, CD79a, IGKC, IGHD, MZB1, MS4A1, CD138, BLK, FAM30A, FCRL2, MS4A1, PNOC, SPIB, TCL1A, TNFRSF17 or combinations thereof; a TLS signature that comprises a gene comprising CCL18, CCL19, CCL21, CXCL13, LAMP3, LTB, MS4A1, or combinations thereof; an effector/memory-like CD8+ T cell signature that comprises a gene comprising CCR7, CD27, CD45RO, CCR7, FLT3LG, GRAP2, IL16, IL7R, LTB, S1PR1, SELL, TCF7, CD62L, or a combination thereof; an HLA-E/CD94 signature that comprises a gene comprising CD94 (KLRD1), CD94 ligand, HLA-E, KLRC1 (NKG2A), KLRB1 (NKG2C) or a combination thereof or a HLA-E/CD94 signature comprising an HLA-E:CD94 interaction level; a NK cell signature that comprises a gene comprising CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2 or a combination thereof; an MHC class II signature that comprises a gene that is an HLA comprising HLA-DMA, HLA-DOA, HLA-DPA1, HLA-DPB1, HLA-DQB1, HLA-DRA, HLA-DRB1, HLA-DRB5 or a combination thereof; or a subset of the above.

In another aspect, provided herein is a drug product which comprises a pharmaceutical composition, wherein the pharmaceutical composition comprises (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein; and at least one pharmaceutically acceptable excipient; and wherein the pharmaceutical composition is indicated for treating cancer in a patient who has a positive test result for a baseline biomarker or an on-treatment biomarker, wherein the baseline biomarker or the on-treatment biomarker comprises a gene signature comprising: a B-cell signature that comprises expression of a gene selected from CD19, CD21, CD22, CD24, CD27, CD38, CD40, CD72, CD3, CD79a, CD79b, IGKC, IGHD, MZB1, TNFRSF17, MS4A1 (cd20), CD138, TNFRSR13B, GUSPB11, BAFFR, AID, IGHM, IGHE, IGHA1, IGHA2, IGHA3, IGHA4, BCL6, FCRLA and combinations thereof; a TLS signature that comprises expression of a gene selected from CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, and combinations thereof; an effector/memory-like CD8+ T cell signature that comprises expression of a gene selected from CCR7, CD27, CD45RO, FLT3LG, GRAP2, IL16, IL7R, LTB, S1PR1, SELL, TCF7, CD62L, PLAC8, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PERI, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, and combinations thereof; an HLA-E/CD94 signature that comprises expression of a gene selected from CD94 (KLRD1), CD94 ligand, HLA-E, and combinations thereof, or an HLA-E:CD94 interaction level; a NK cell signature that comprises expression of a gene selected from CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, NCAM1, and combinations thereof; an MHC class II signature that comprises expression of a gene selected from HLA-DMA, HLA-DOA, HLA-DPA1, HLA-DPB1, HLA-DQB1, HLA-DRA, HLA-DRB1, HLA-DRB5 and combinations thereof; or a combination or subset of any of the above.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended embodiments. A better understanding of the features and advantages of the present invention will be obtained by reference to the following detailed description that sets forth illustrative embodiments, in which the principles of the invention are utilized, and the accompanying drawings (also “FIG.” and “Fig.” herein), of which:

FIG. 1 is an exemplary schematic of treatment regimen and assessment schedule using neoantigen peptide vaccine and nivolumab. Abbreviations used: NSCLC, non-small cell lung cancer.

FIG. 2 is a graph showing an 18-gene TIS signature that measures a pre-existing but suppressed adaptive immune response within tumors in samples from pre-treated melanoma patients with and without DCB [left panel]. The right panel depicts an exemplary graph of tumor mutational burden (TMB) within pre-treatment tumor samples from melanoma patients with and without DCB.

FIG. 3A depicts an exemplary graph of a CD8+ T cell signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). The CD8+ T cell signature is increased in melanoma patients with DCB.

FIG. 3B depicts an exemplary graph of a memory and/or effector-like TCF7+ CD8+ T cell signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine) The TCF7+ CD8+ T cell signature is increased in melanoma patients with DCB. The memory and/or effector-like TCF7+ CD8 T cell associated signature was derived from CD8+ T cell sub-clusters that express genes consistent with a memory- and/or effector-like phenotype and express the stem-like transcription factor TCF7. Higher expression of this gene signature is associated with DCB and predicts outcome of metastatic melanoma patients.

FIG. 4A depicts a representative series of photomicrographs of multiplexed immunohistochemistry of melanoma tumor biopsies. Markers for CD8+ T cells, TCF7, tumor cells (S100), and nuclear stain DAPI were simultaneously used to examine expression of TCF7 in CD8+ T cells in patients with DCB and no DCB at pre-treatment, pre-vaccine, and post-vaccine timepoints. A representative patient from each cohort is shown. Scale bar represents 50 μm

FIG. 4B depicts a graph showing the differential levels of TCF7+ CD8+ T cell signature between DCB and no-DCB patient samples before (pre-treatment) and after vaccination with a neoantigen peptide vaccine (post-vaccine).

FIG. 4C depicts two photomicrographs of the same patients presented in FIG. 4A, representing multiplex immunohistochemistry for tumor marker S100, CD8+ T cell marker CD8, the transcription factor TCF7 and nucleus stain DAPI on tumor biopsies at pre-treatment.

FIG. 5A depicts graphs showing a comparison of B cell signatures of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). The data shows that higher B cell signatures are associated with DCB in melanoma patients. Patients with DCB have a higher IO360 B cell signature at pre-treatment and over the course of treatment.

FIG. 5B depicts a heat map of individual gene expression of B cell-associated genes of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). Expression of individual genes associated with B cells is also increased in patients with DCB over the course of treatment

FIG. 6 depicts graphs showing a comparison of a TLS signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). The data shows that the TLS signature is associated with patients who have DCB. The TLS signature was derived and calculated using genes associated with TLS, including chemokines, cytokines, and specific cell populations.

FIG. 7 depicts a graph showing that the TLS signature highly correlates with the B cell signature within the TME and is independent of lymph node biopsies.

FIG. 8A depicts a representative series of photomicrographs of multiplexed immunohistochemistry of melanoma tumor biopsies. Markers for B cells (CD20), T cells (CD3), tumor cells (S100), and nuclear stain DAPI were simultaneously used to examine TLS in a melanoma patient with DCB and a melanoma patient with no DCB at pre-treatment, pre-vaccine and post-vaccine timepoints. Clusters or individual B cells are indicated by white arrows, and T cells are denoted by yellow arrows. Scale bar represents 50 μm.

FIG. 8B depicts graphs showing a comparison of B cell signatures of melanoma patients (with DCB and without DCB) prior to receiving treatment (left graph, pre-treatment), and after treatment with a neoantigen peptide vaccine (right graph, post-vaccine).

FIG. 8C depicts two photomicrographs of the same patients presented in FIG. 8A, representing multiplex immunohistochemistry for tumor marker S100, B-cell marker CD20, T-cell marker CD3 and nucleus stain DAPI on tumor biopsies before vaccination.

FIG. 9 depicts graphs showing a comparison of a cytotoxic CD56dim NK cell signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). Gene expression associated with cytotoxic CD56dim NK cells is higher in patients with DCB. Expression of genes associated with cytolytic CD56dim NK cells is increased in patients with DCB post-treatment (post-vaccine) and is significantly higher than patients with no DCB at the post-vaccine time point. Cytolytic CD56dim NK cells can recognize and kill tumor cells through ADCC, suggesting a potential role with B cells, and direct cell lysis via NCRs.

FIG. 10A depicts graphs showing a comparison of a MHC-II gene signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). MHC Class II gene expression is associated with DCB. Patients with DCB have higher expression of MHC Class II, and this expression at pre-treatment is predictive of outcome.

FIG. 10B depicts photomicrographs that shows MHC-II expression in tumor biopsies at pre-treatment in a patient with DCB and a patient without DCB. MHC Class II is expressed on tumor cells in patients with DCB.

FIG. 11 depicts graphs showing a comparison of an inhibitory ligand B7-H3 signature of melanoma patients (with DCB and without DCB) prior to receiving treatment (left graph, pre-treatment), after nivolumab treatment (middle graph, pre-vaccine), and after treatment with nivolumab and a neoantigen peptide vaccine (right graph, post-vaccine). B7-H3 gene expression is higher in patients with no DCB.

FIG. 12A depicts exemplary data showing the percent change in the total number of target lesions in melanoma subjects over time after nivolumab treatment and after treatment with a neoantigen peptide vaccine.

FIG. 12B is an exemplary graph that shows the percent of vaccine peptides administered per patient that generated an immune response in the patient.

FIG. 13A depicts a graph of the number of spot forming cells per 1×10⁶ PBMCs from subjects prior to treatment with vaccine and after treatment with vaccine.

FIG. 13B is an exemplary depiction of a FACS analysis of percentage of neoantigen-specific CD4− T cells and neoantigen-specific CD8− T cells from samples from the subjects shown in FIG. 13A treated with vaccine.

FIG. 14A is an exemplary depiction of a FACS analysis of tetramer positivity before and after treatment with a neoantigen peptide vaccine.

FIG. 14B depicts the number of sequence reads (normalized) of neoantigen-specific TCR prior to receiving treatment, after nivolumab treatment, and after treatment with nivolumab and a neoantigen peptide vaccine.

FIG. 14C is an exemplary graph depicting percent Caspase 3 positive A375-B51-01 cells after stimulation with PBMCs from a patient prior to treatment and transduced with a mutant RICTOR peptide-specific TCR.

FIG. 15 shows an exemplary pathology scores in biopsies taken from melanoma patients (with DCB and without DCB) prior to receiving treatment (left graph), after nivolumab treatment (middle graph), and after treatment with nivolumab and a neoantigen peptide vaccine (right graph).

FIG. 16A depicts results showing the percentage of naïve T cells (CD19−, CD3+, CD8+, CD62L+ and CD45RA+) as percent of total CD8+ T cells (bottom right) in a peripheral blood sample from melanoma patients (with DCB and without DCB) prior to receiving treatment, after nivolumab treatment, and after treatment with nivolumab and a neoantigen peptide vaccine. The results indicate that treatment of melanoma patients with a naïve T cell population of greater than 20% of total CD8+ T cells may be less likely to receive durable clinical benefit. The results indicate that treatment of melanoma cancer patients with a naïve T cell population of 20% or less of total CD8+ T cells may be more likely to receive durable clinical benefit.

Also depicted are results showing the percentage of effector memory T cells (CD19−, CD3+, CD8+, CD62L− and CD45RA−) as percent of total CD8+ T cells (bottom left) in a peripheral blood sample from melanoma patients (with DCB and without DCB) prior to receiving treatment, after nivolumab treatment, and after treatment with nivolumab and a neoantigen peptide vaccine. The results indicate that melanoma patients with an effector memory T cell population of less than 40% of total CD8+ T cells may be less likely to receive durable clinical benefit. The results indicate that treatment of melanoma cancer patients with an effector memory T cell population of 40% or greater of total CD8+ T cells may be more likely to receive durable clinical benefit.

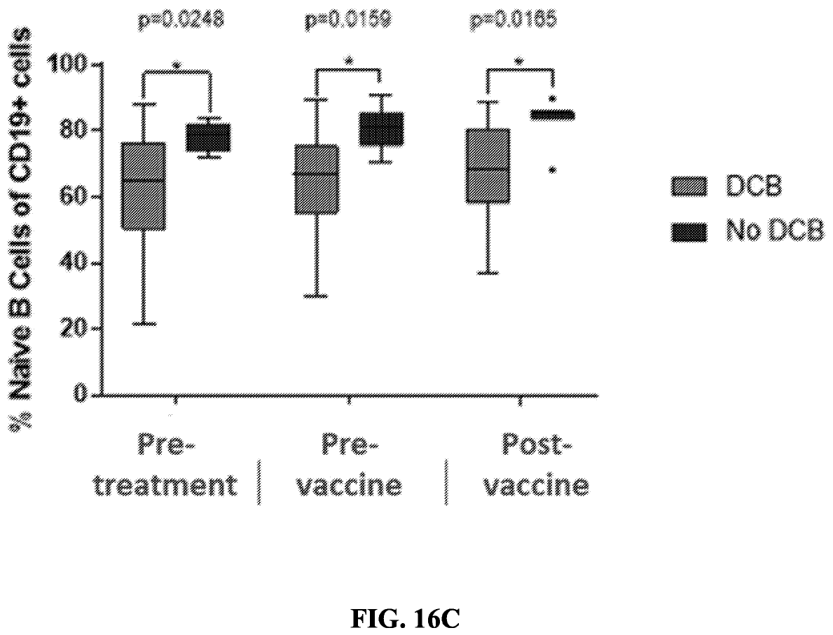

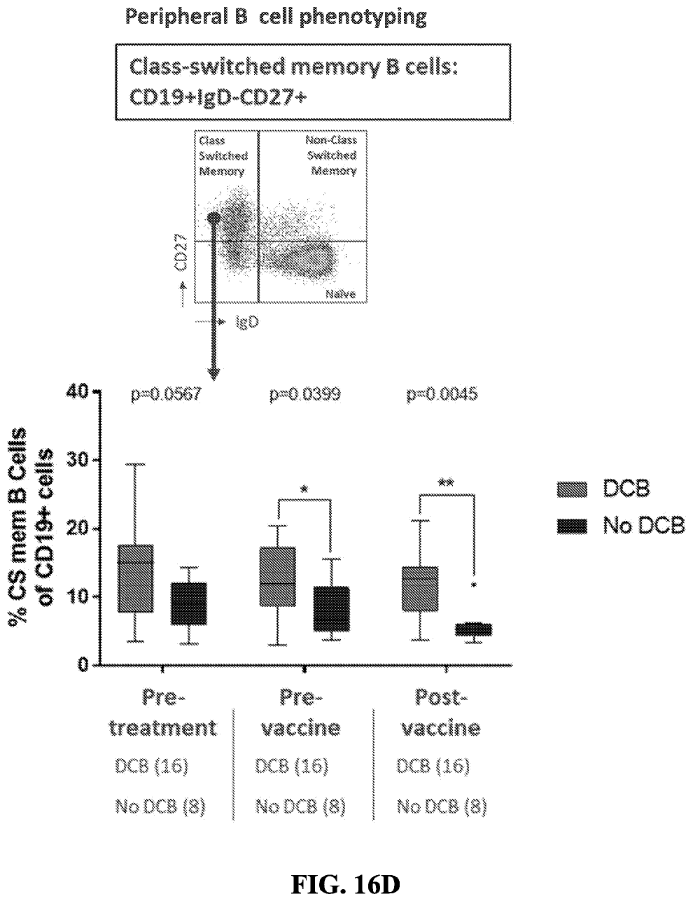

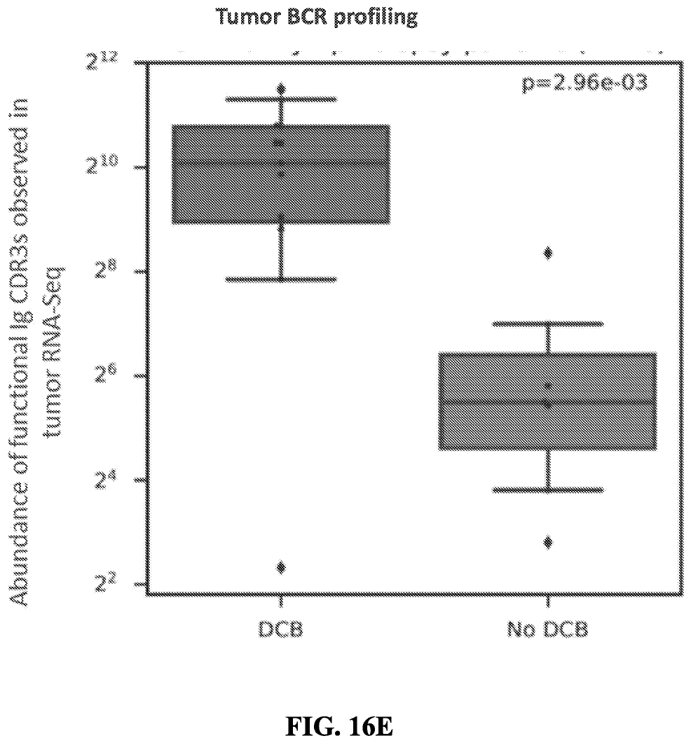

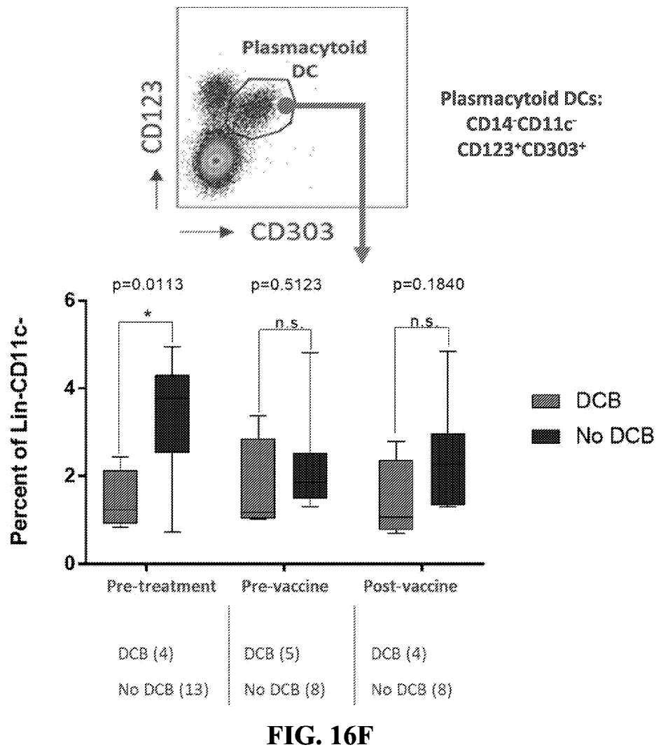

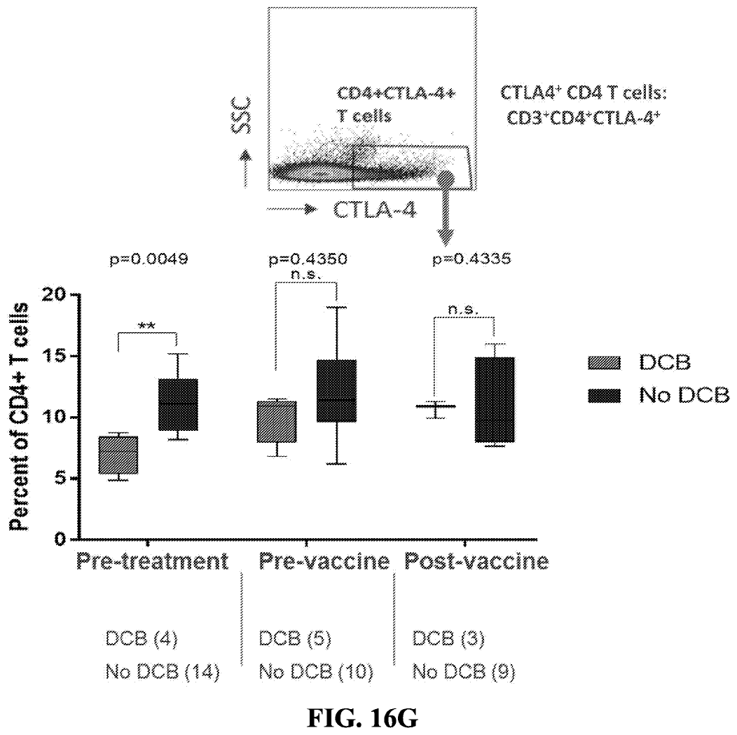

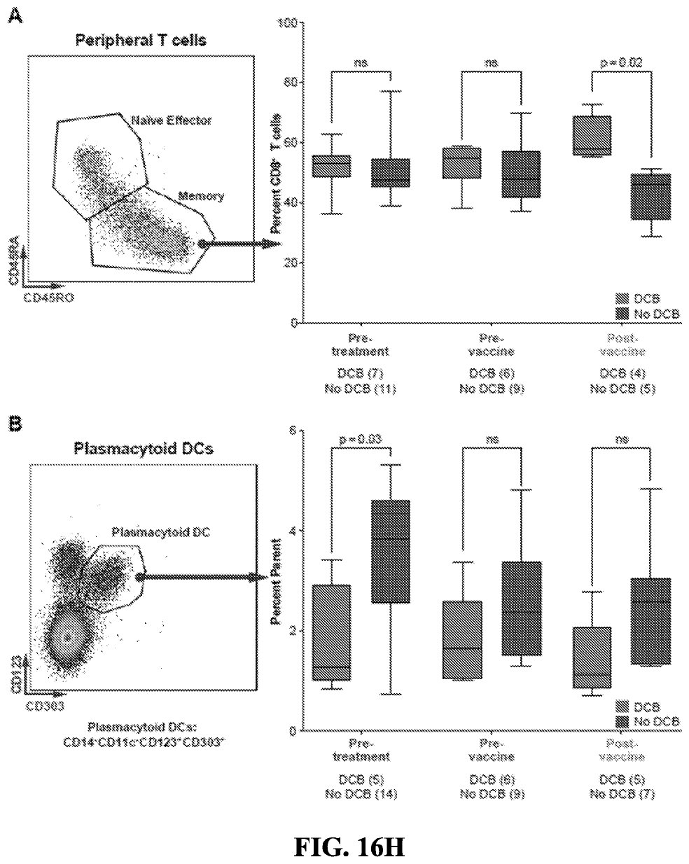

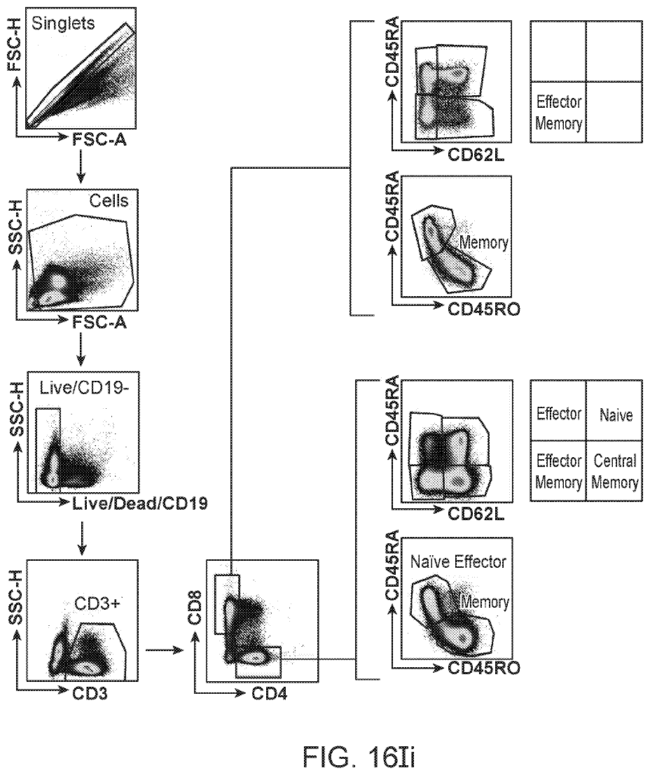

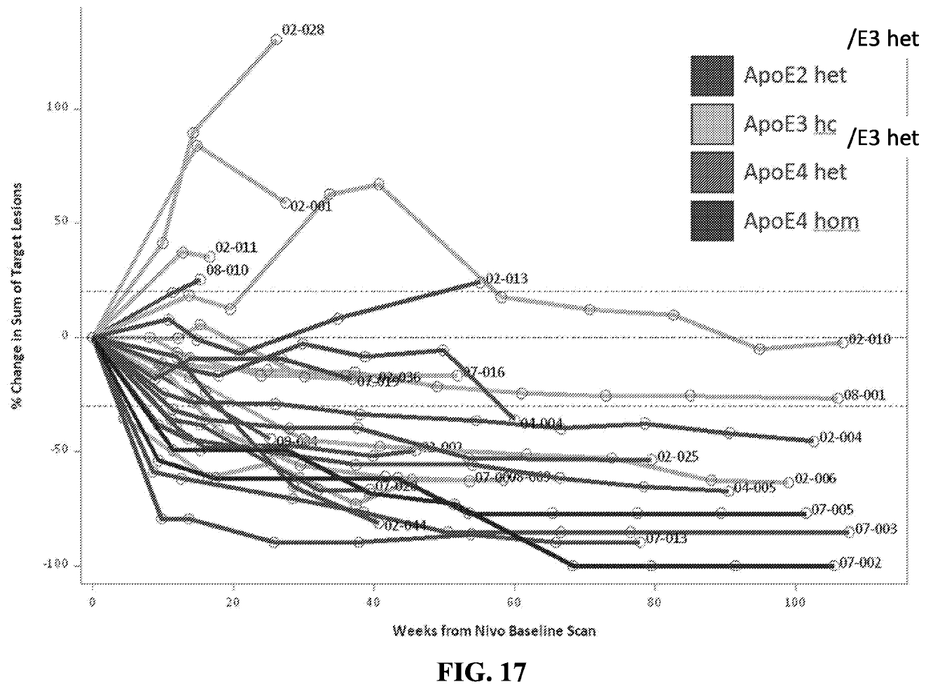

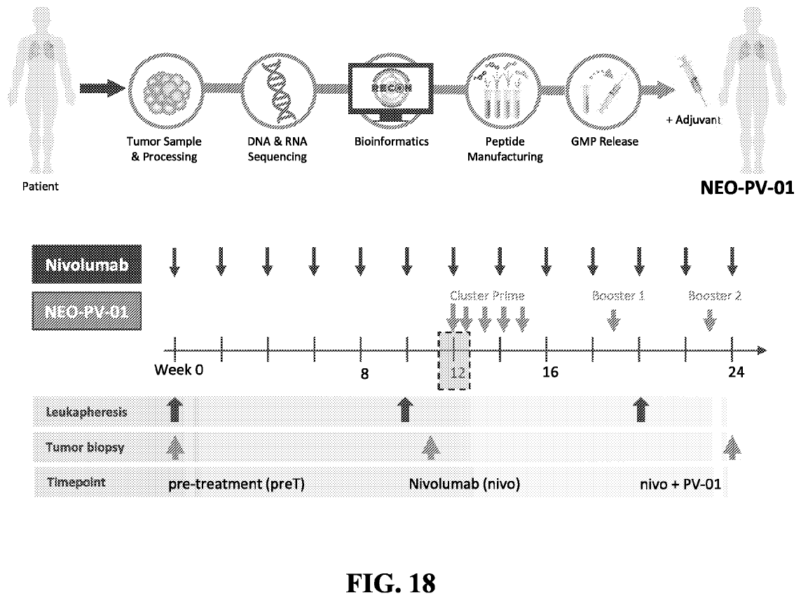

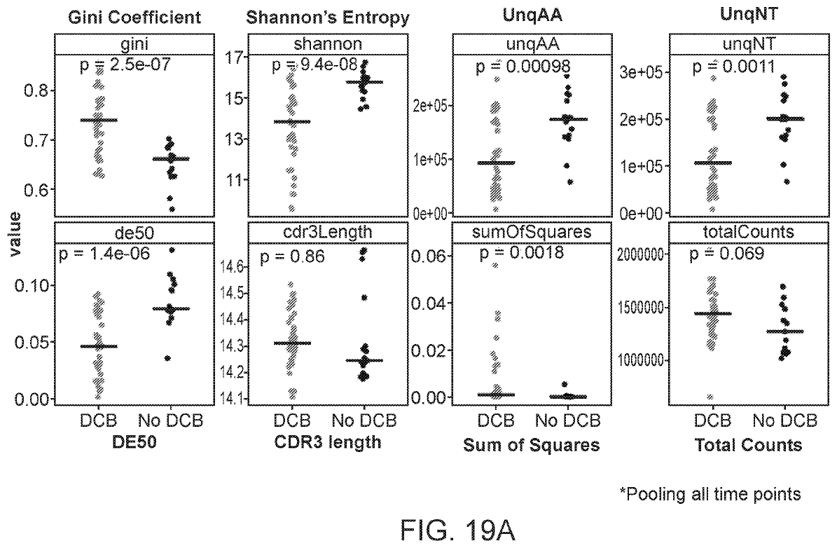

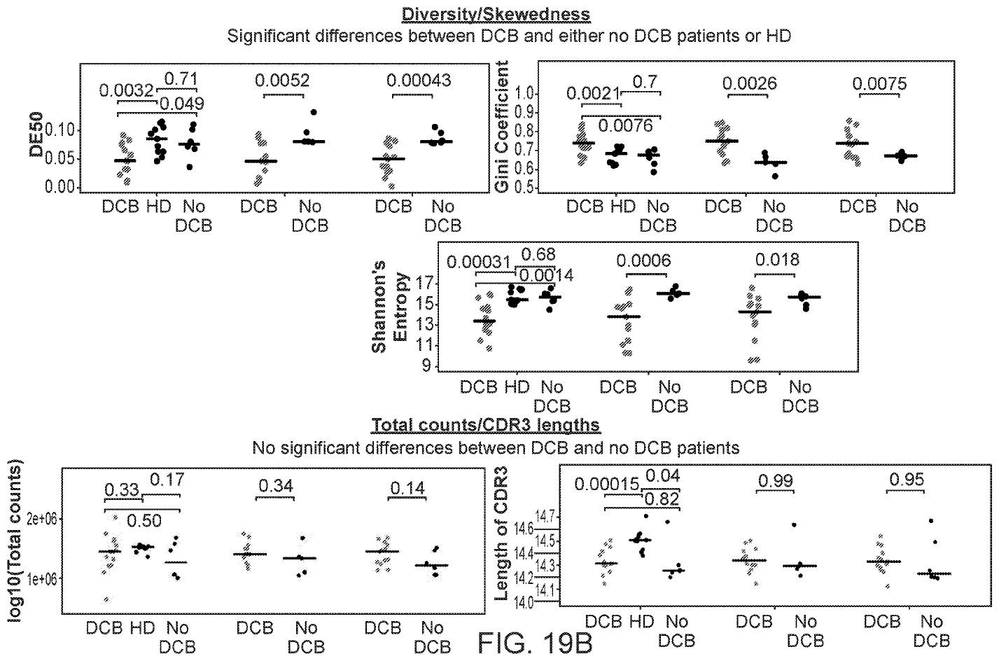

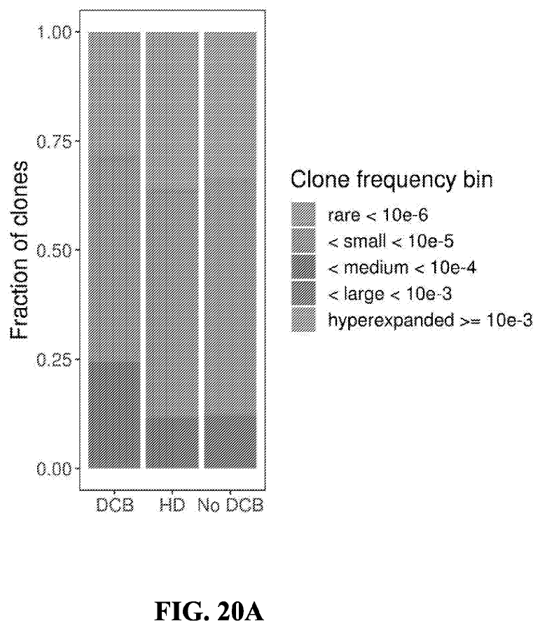

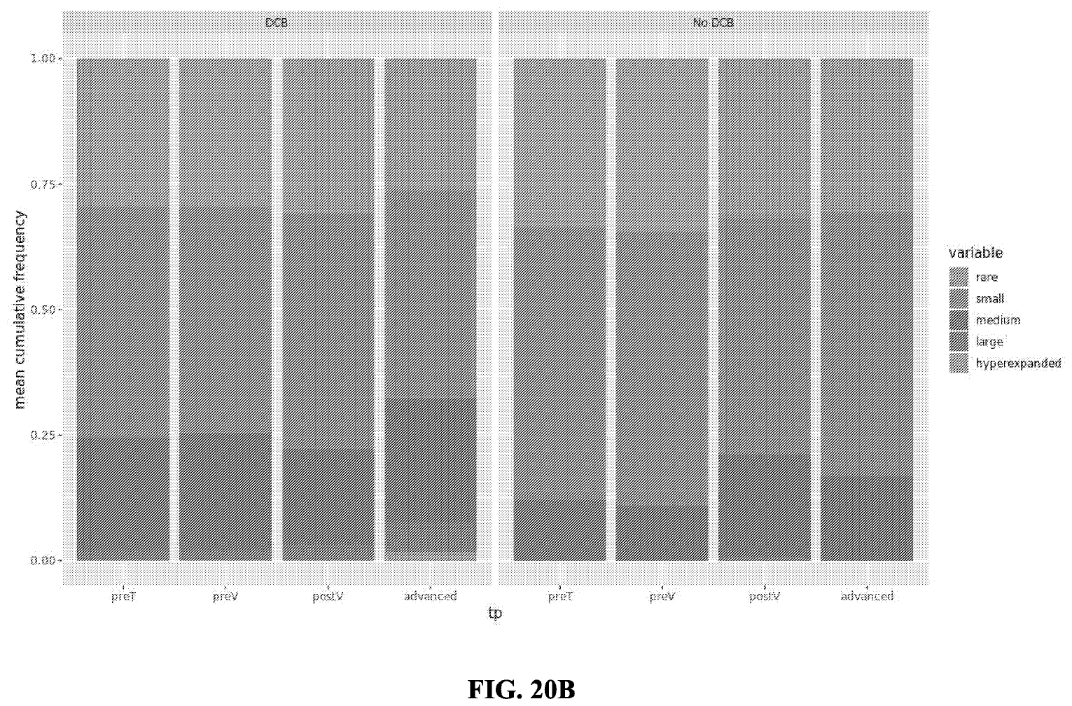

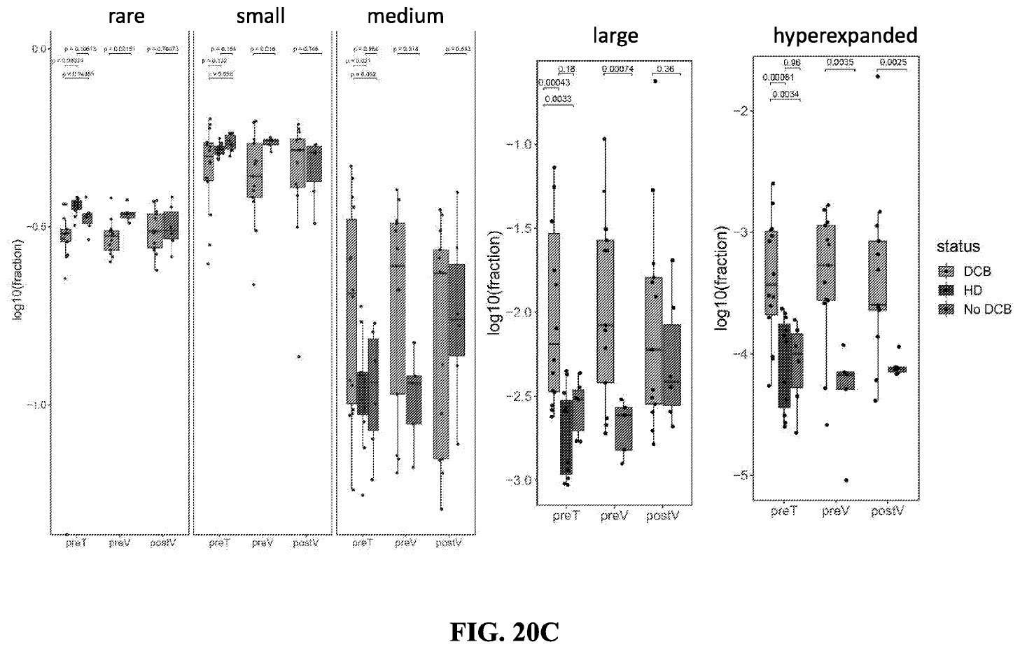

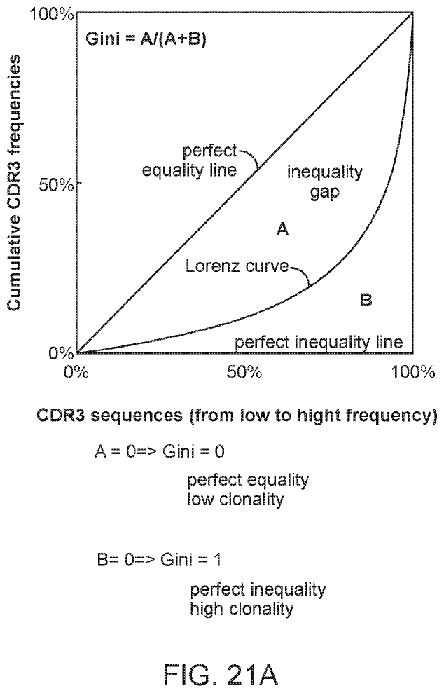

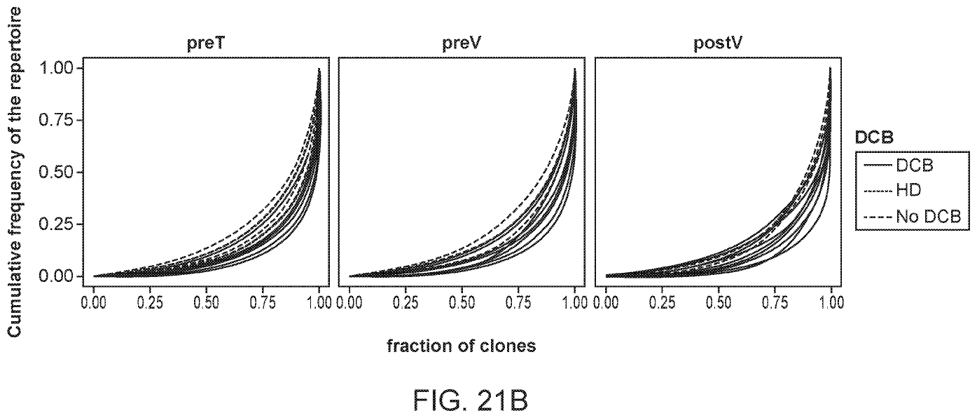

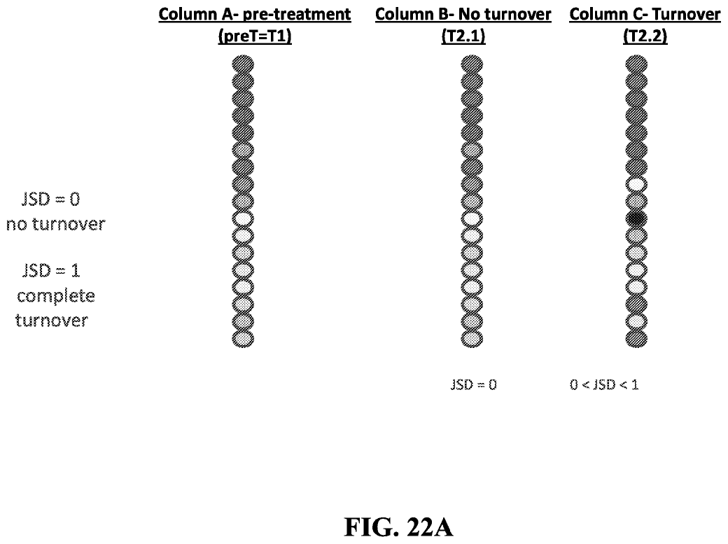

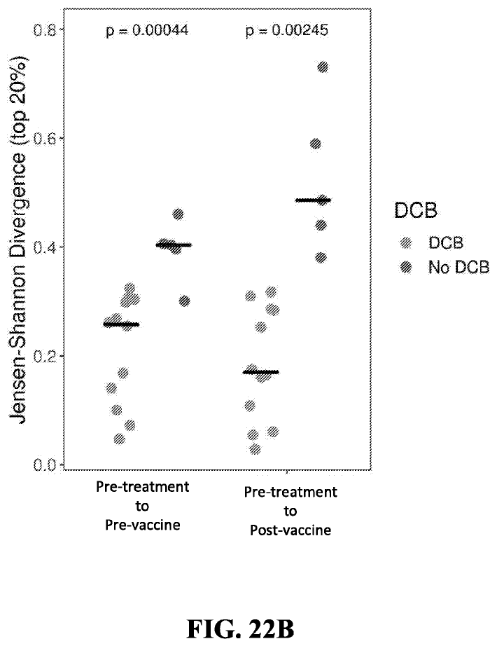

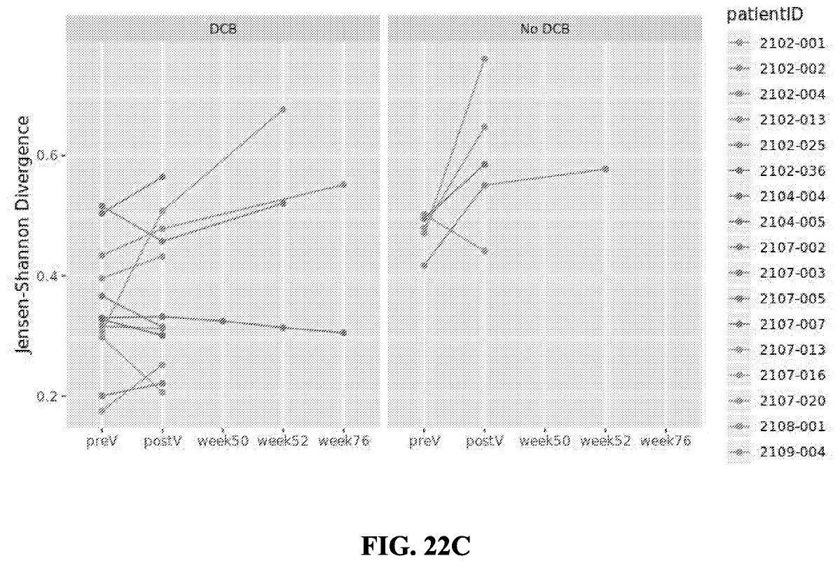

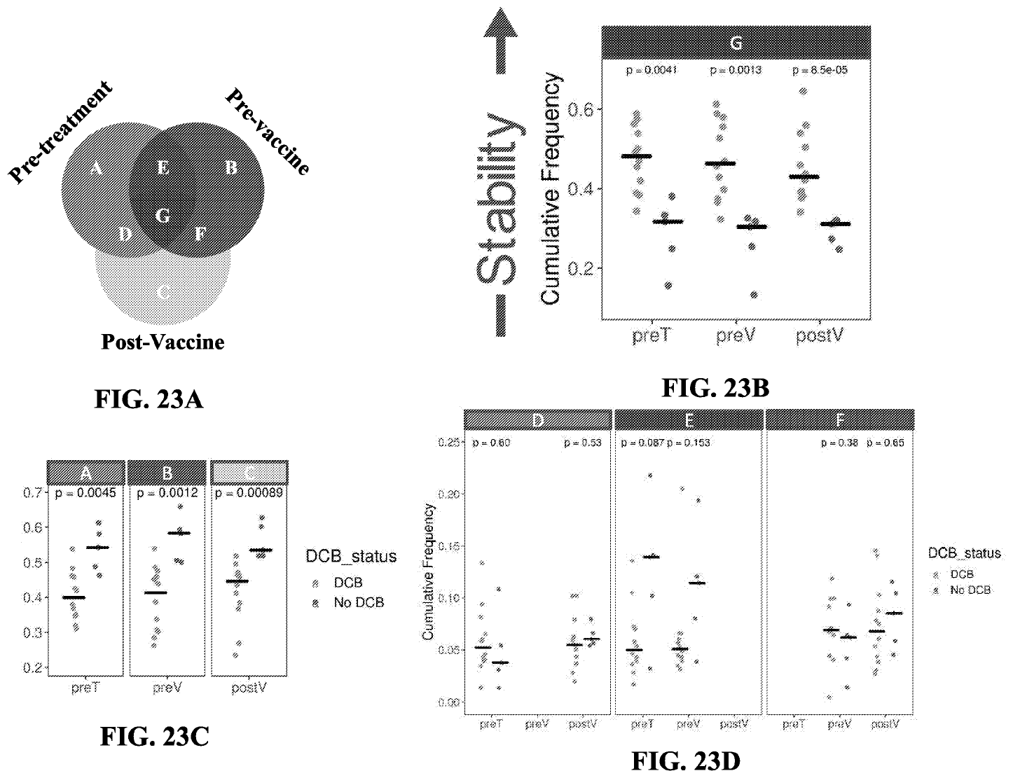

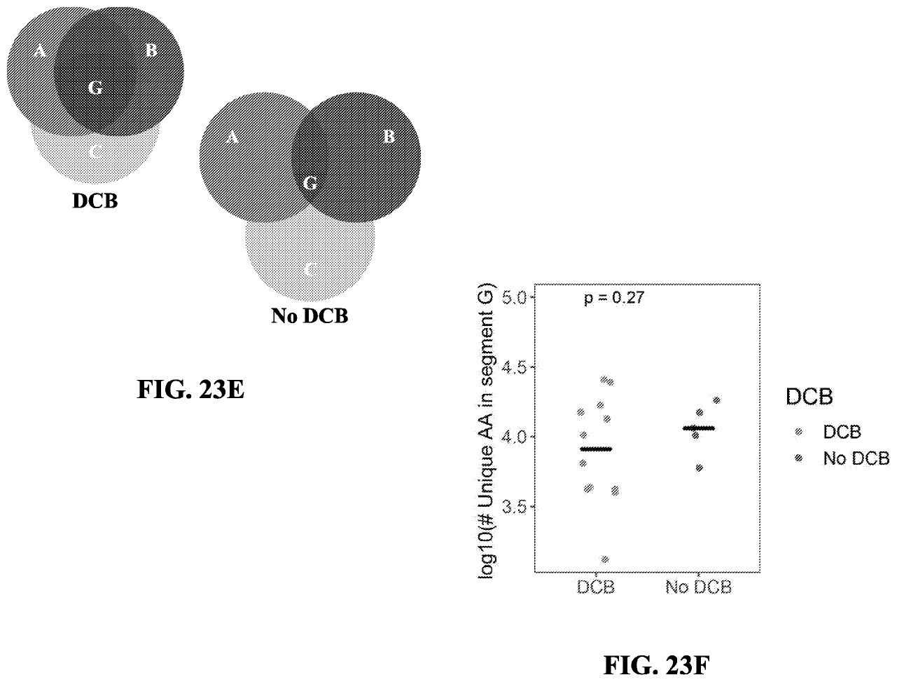

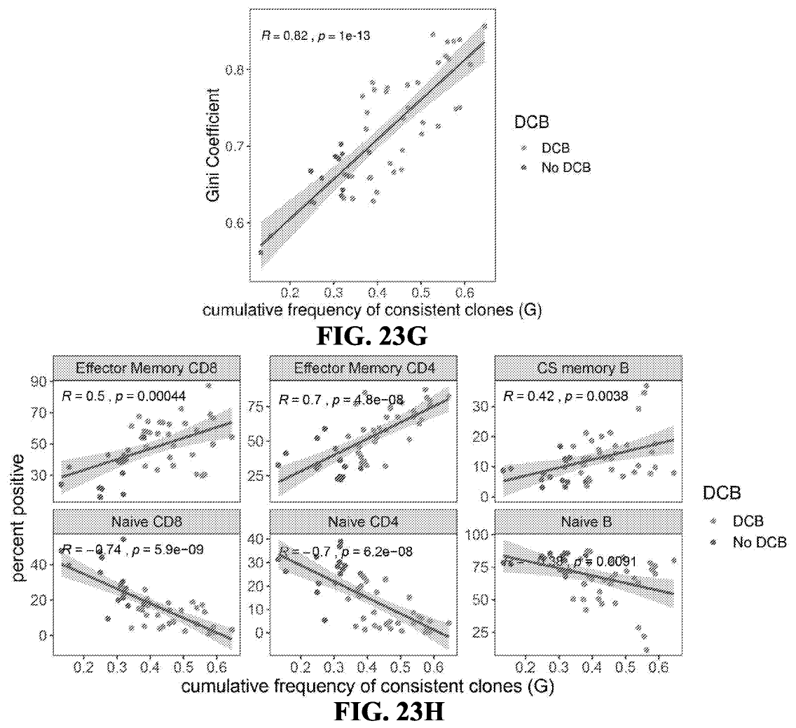

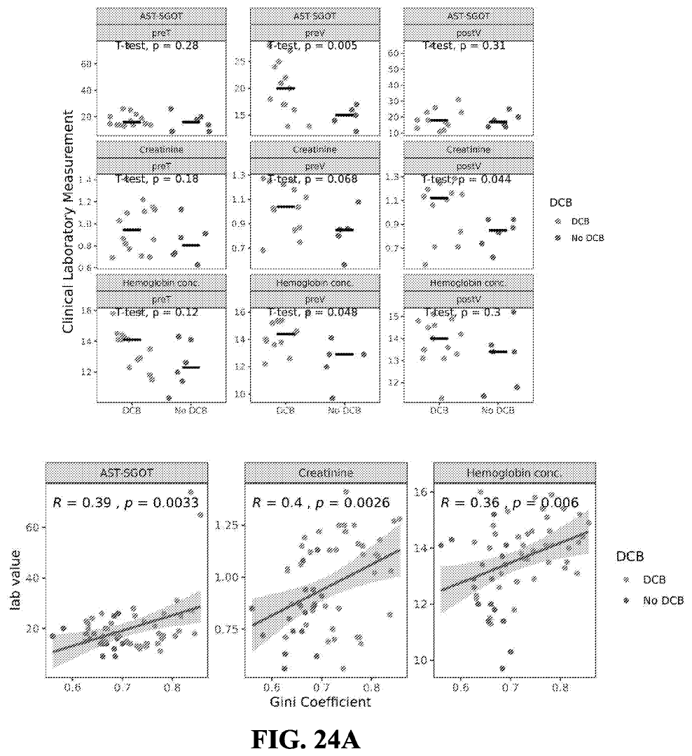

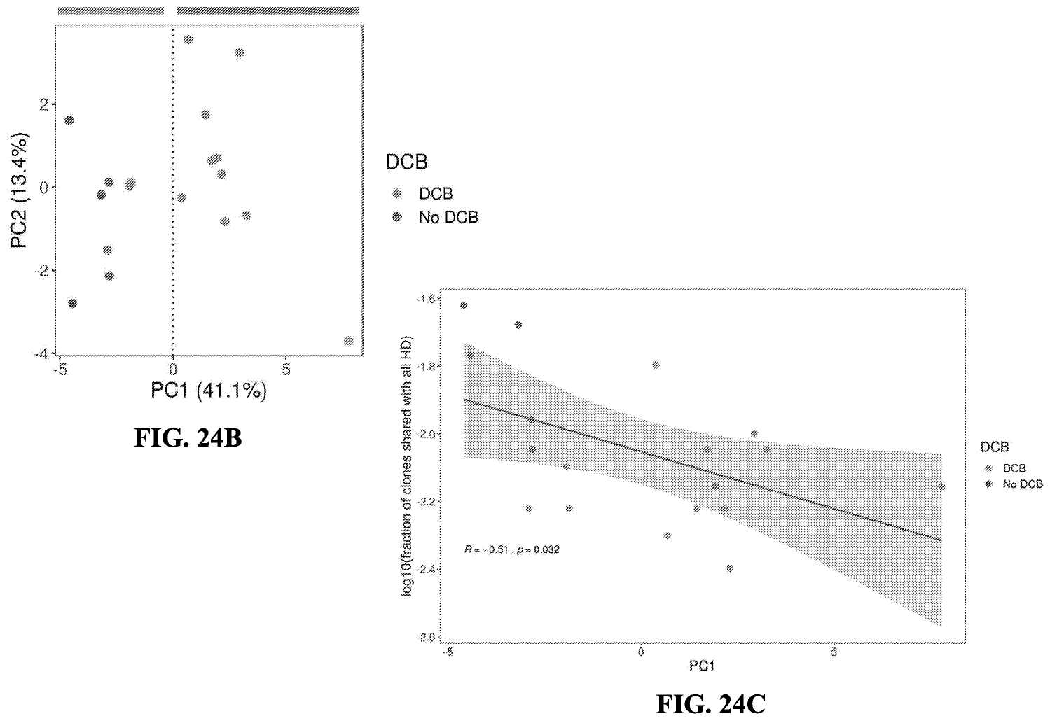

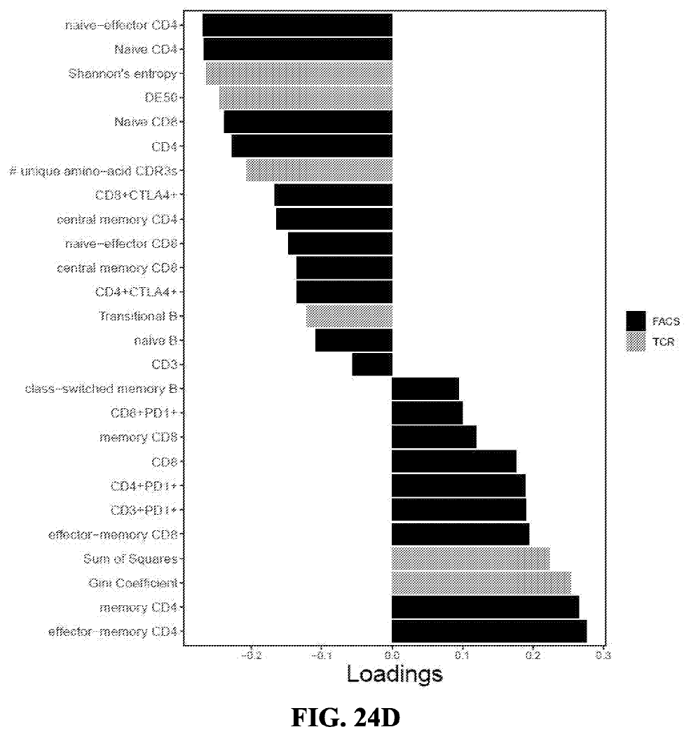

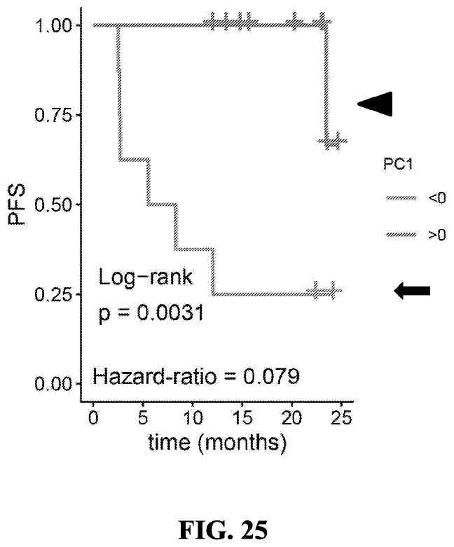

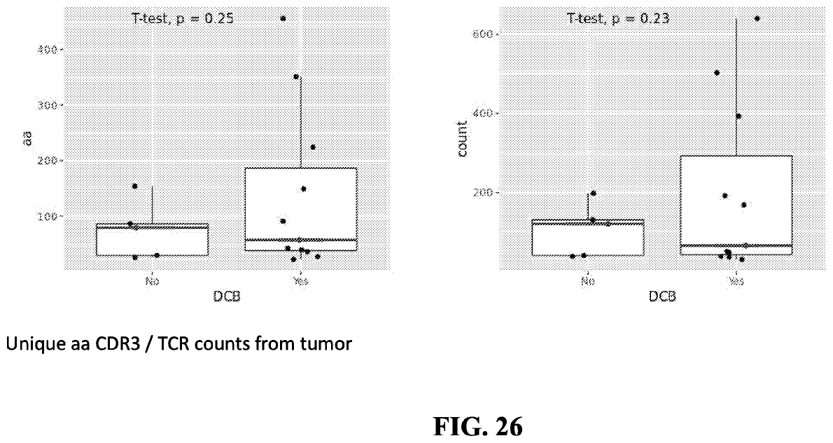

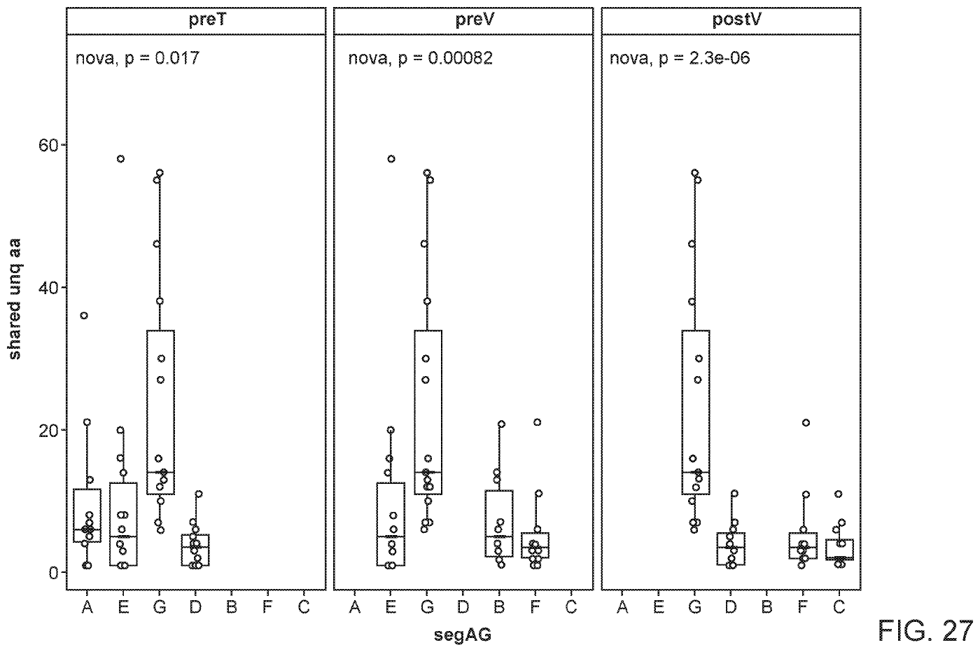

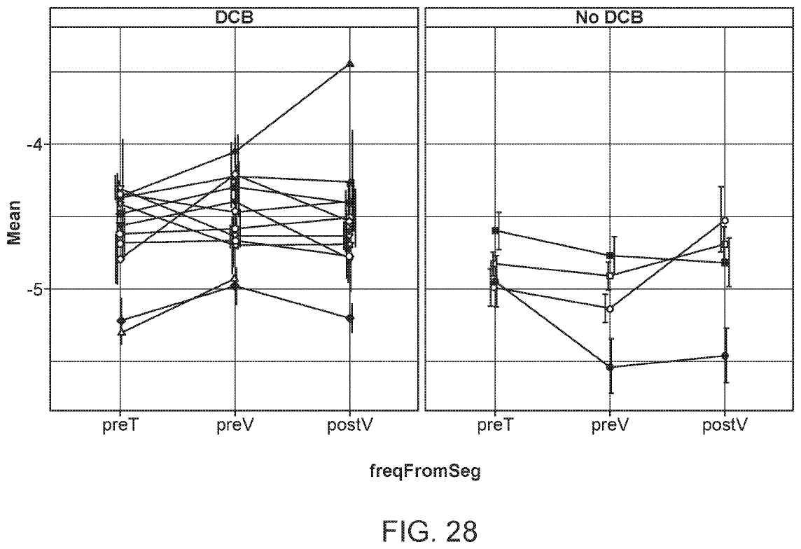

FIG. 16B depicts an exemplary graph of a peripheral TCR repertoire analysis showing the Gini-coefficient in a peripheral blood sample from melanoma patients (with DCB and without DCB) prior to receiving treatment. The results show that a more uneven TCR frequency distribution in patients with DCB may indicate a more clonal T cell population.

FIG. 16C depicts results showing the percentage of naïve B cells (CD56−, CD3−, CD14−, CD19+, IgD+ and CD27−) as a percent of total CD19+ B cells in a peripheral blood sample from melanoma patients (with DCB and without DCB) prior to receiving treatment (left graph), after nivolumab treatment (middle graph), and after treatment with nivolumab and a neoantigen peptide vaccine (right graph). The results indicate that treatment of melanoma patients with a naïve B cell population of greater than 70% of total CD19+ B cells may be less likely to receive durable clinical benefit. The results indicate that treatment of melanoma patients with a naïve B cell population of 70% or less of total CD19+ B cells may be more likely to receive durable clinical benefit.

FIG. 16D depicts results showing the percentage of class-switched memory B cells (CD19+, IgD−, CD27+) as a percent of total CD19+ B cells in a peripheral blood sample from melanoma patients (with DCB and without DCB) prior to receiving treatment (left graph), after nivolumab treatment (middle graph), and after treatment with nivolumab and a neoantigen peptide vaccine (right graph). The results show that higher levels of class switched memory B cells were seen in patients with durable clinical benefit compared to patients with no durable clinical benefit. The results indicate that treatment of melanoma patients with a class-switched memory B cell population of greater than 10% of total CD19+ B cells may be more likely to receive durable clinical benefit. The results indicate that treatment of melanoma patients with a class-switched memory B cell population of 10% or less of total CD19+ B cells may be less likely to receive durable clinical benefit.

FIG. 16E depicts results showing the abundance of functional Ig CDR3s observed by RNA-seq from cells of TME samples from melanoma patients (with DCB and without DCB) prior to receiving treatment. These exemplary results show that higher levels of functional B cells in the TME were seen in patients with durable clinical benefit compared to patients with no durable clinical benefit. These exemplary results indicate that treatment of melanoma patients with, for example, less than 2\hat{ }7 functional Ig CDR3s (e.g., as observed by RNA-seq) from cells of TME samples may be less likely to receive durable clinical benefit. These exemplary results indicate that treatment of melanoma patients with, for example, 2\hat{ }7 or more functional Ig CDR3s (e.g., as observed by RNA-seq) from cells of TME samples may be more likely to receive durable clinical benefit.

FIG. 16F depicts results showing the percentage of plasmacytoid DC population (CD3−, CD19−, CD56−, CD14−, CD11c−, CD123+ and CD303+) as a percent of total Lin−/CD11c− cells in a peripheral blood sample from NSCLC patients (with DCB and without DCB) prior to receiving treatment (left graph), after nivolumab treatment (middle graph), and after treatment with nivolumab and a neoantigen peptide vaccine (right graph). The results indicate that treatment of NSCLC patients with a plasmacytoid DC population of greater than 3% of total Lin−/CD11c− cells may be less likely to receive durable clinical benefit. The results indicate that treatment of NSCLC patients with a plasmacytoid DC population of 3% or less of total Lin−/CD11c− cells may be more likely to receive durable clinical benefit.

FIG. 16G depicts results showing the percentage of CTLA4+ CD4 T cells (CD3+, CD4+, CTLA4+) as a percent of total CD4+ T cells in a peripheral blood sample from NSCLC patients (with DCB and without DCB) prior to receiving treatment (left graph), after nivolumab treatment (middle graph), and after treatment with nivolumab and a neoantigen peptide vaccine (right graph). The results show that NSCLC patients with DCB (9-month PFS) have lower levels of CTLA4+ CD4 T cells than NSCLC patients without DCB. The results indicate that treatment of NSCLC patients with a CTLA4+ CD4 T cell population of greater than 9% of total CD4+ T cells may be less likely to receive durable clinical benefit. The results indicate that treatment of NSCLC patients with a CTLA4+ CD4 T cell population of 9% or less of total CD4+ T cells may be more likely to receive durable clinical benefit.

FIG. 16H depicts exemplary data showing the percentage of memory CD8+ T cells (CD3+, CD8+, CD45RA−, CD45RO+) as a percent of total CD8+ T cells results from (with DCB and without DCB) prior to receiving treatment, after nivolumab treatment, and after treatment with nivolumab and a neoantigen peptide vaccine. The results show that patients who receive durable clinical benefit as defined by progression free survival 6 months post initiation of treatment had higher levels of memory T cells when compared to patients who progressed specifically in the post vaccine time point. This marker could be used as mechanistic marker for evaluating vaccine effect post treatment. The results indicate that bladder cancer patients with a memory CD8+ T cells population of less than 40% or less than 55% of total CD8+ T cells at the post vaccine time point are less likely to receive durable clinical benefit. The results indicate that bladder cancer patients with a memory CD8+ T cells population of 40% or more or 55% or more of total CD8+ T cells at the post vaccine time point are more likely to receive durable clinical benefit.

FIG. 16Ii depicts an exemplary cell gating strategy for CD4 and CD8 T cell subpopulations using the FlowJo software. Gating was performed in the sequence depicted, starting with singlets and cells, followed by gating on live, CD19− cells, then CD3+, CD4+ vs. CD8+, and finally CD62L+vs CD45RA+ or CD45RO vs CD45RA.

FIG. 16Iii depicts an exemplary cell gating strategy for B cell subpopulations using the FlowJo software. Gating was performed in the sequence depicted, starting with cells and singlets, followed by gating on live, CD3/CD14/CD56− cells, then CD19+, and finally CD27 vs IgD.

FIG. 17 depicts exemplary data showing the percent change in the total number of target lesions in melanoma subjects with the indicated ApoE genotype over time after nivolumab treatment and after treatment with a neoantigen peptide vaccine.

FIG. 18 depicts a schematic diagram showing treatment regimen and assessment schedule using neoantigen peptide vaccine and nivolumab (nivo). Nivolumab alone was administered as indicated by blue arrows in the “Nivolumab” timeline starting at week 0 and occurring every 2 weeks thereafter. Vaccine was administered starting at Week 12 as 5 priming doses (“Cluster Prime”), followed by a “Booster 1” dose at week 19 and a “Booster 2” dose at week 23 as indicated by green arrows in the “NEO-PV-01” timeline. Leukapheresis samples were obtained prior to start of administration of therapy at Week 0, (“Pretreatment (preT)”), Week 10, and Week 20 as indicated by red arrows in the “Leukapheresis timeline”.)

FIGS. 19A-19B depict representative data from analysis of TCR repertoire diversity and frequency distribution in samples from melanoma patients who experienced durable clinical benefit upon treatments (DCB), or who did not show DCB (No DCB); measured by Gini Coefficient (Gini), DE50, Sum of Squares and Shannon entropy (Shannon), the number of unique nucleotide CDR3 (unqNT) and unique amino acid CDR3 (unqAA) sequences. In addition, the CDR3 length and counts are shown. FIG. 19A shows values for all time points pooled together. FIG. 19B shows values at indicated times, PreT=Pretreatment (Week 0 pre-Nivolumab); PreV=Pre-vaccine administration; PostV=post-vaccine administration. These values were calculated for healthy donors (HD), which was labeled as a preT measurement. UnqNT, Unique nucleotides; UnqAA, Unique amino acids; NS, non-significant.

FIGS. 20A-20C depict representative data from analysis of TCR repertoire diversity based on TCR frequency categories in samples from melanoma patients who experience durable clinical benefit upon treatments (DCB), or who do not (No DCB), and healthy donors (HD). Each TCR clone was assigned a size designation/category based on its frequency (rare, small, medium, large and hyperexpanded). FIG. 20A depicts representative data showing average values of TCR repertoire frequency sizes in all time points pooled. Healthy donor samples were treated as preT. FIG. 20B shows mean frequency values (mean cumulative frequency) in DCB and No DCB patients at individual analysis timepoints (tp) for all five size categories. FIG. 20C shows frequency values (on a log 10 scale) in DCB and No DCB patients and HD at individual analysis timepoints (tp) for all size-categories. Indicated timepoints: PreT=Pretreatment (Week 0 pre-Nivolumab); PreV=pre-vaccine administration; PostV=post-vaccine administration; Advanced, later than 52 weeks.

FIGS. 21A-21B depict representative data showing TCR repertoire diversity as indicated by inequality assessments. FIG. 21A shows exemplary depiction of inequality by Gini coefficient and Lorenz curve. FIG. 21B shows data obtained from DCB and No DCB patient samples, and healthy donors (HD) at the indicated time points, PreT=Pretreatment (Week 0 pre-Nivolumab); PreV=Pre-vaccine administration; PostV=post vaccine administration. DCB patient samples had lower diversity and therefore lower equality, as indicated in the Lorenz curves.

FIGS. 22A-22C depict representative data showing TCR repertoire stability as indicated by Jensen-Shannon Divergence (JSD). FIG. 22A is a graphical representation that explains the principle behind a JSD data range. As indicated in FIG. 22A, a mathematical difference between an exemplary T cell repertoire shown in Column A (T1) to another T cell repertoire shown in Column B (T2.1) indicates no turnover of T cell clones, and therefore, JSD is 0. A mathematical difference between an exemplary T cell repertoire shown in Column A (T1) to another T cell repertoire shown in Column C (T2.2) indicates some T cell clone turnover, but not all, and therefore, JSD is greater than 0, but less than 1. FIG. 22B shows representative JSD values in DCB and No DCB peripheral blood samples at either pre-vaccine (preV in FIG. 22B, left) or post-vaccine (postV in FIG. 22B, right) timepoints compared to Week 0 pre-Nivolumab patient samples, illustrating that in both cases, there is a significant decrease in JSD values in DCB patients (versus no DCB patients), thereby demonstrating lower turnover of DCB T cell repertoires than the turnover in T cell repertoires of No DCB patients. FIG. 22C shows representative JSD values of samples from individual patients at either pre-vaccine or post-vaccine timepoints compared to Week 0 pre-Nivolumab treatment, shown over an extended time period (i.e., up to week 76) for the available patients. Longer-term turnover of T Cell repertoires may be assessed with additional forthcoming patient data.

FIGS. 23A-23H depict representative data showing TCR repertoire stability using a Venn diagram (on FIG. 23A) of TCR clonotypes at indicated time points, PreT=Pretreatment (Week 0 pre-Nivolumab); PreV=Pre-Vaccine administration; PostV=post-vaccine administration. The Venn diagram on FIG. 23A shows 7 resulting segments (i.e., A through G) possible for 3 overlapping time points; each time point spanning 4 segments (e.g., A, E, D, G in the Pre-treatment patient sample). FIGS. 23B-23D show the cumulative frequency of T cells clones found in each segment of the Venn diagram, with respect to each time point. More specifically, FIG. 23B shows representative data of cumulative TCR frequencies of clones within the G (overlap of all timepoints) segment of the Venn diagram, at each time-point, depicting change of G cumulative frequencies at the time points, PreT=Pretreatment (Week 0 pre-Nivolumab); PreV=pre-vaccine administration; PostV=post-vaccine administration in DCB and No DCB patients. FIG. 23C shows representative data of cumulative TCR frequencies of clones detected at a single time point alone within segments A, B and C of the Venn diagram, at each respective time-point. FIG. 23D shows representative data of cumulative TCR frequencies of clones detected at two specific time-points within segments D, E and F) of the Venn diagram, at the respective time-point. This illustrates that the cumulative frequency of T cell clones detected at all 3 time points are higher in DCB patients than in No DCB patients. The Venn diagram appearing on the left side of FIG. 23E is a visual representation of the DCB patient repertoires which have an increased G frequency relative to No DCB patients; whereas the Venn diagram appearing to the right of FIG. 23E is a visual representation of the No DCB patient repertoires which have a decreased G frequency relative to DCB patient repertoires. FIG. 23F shows data representing the number of unique amino acids (AA) in the G overlap region for DCB and No DCB patients. FIG. 23G, shows Gini Coefficient values of each patient as a function of the cumulative frequency of segment G, which represents persistent clones only, over the three time-points. Color indicates DCB/No DCB. Repertoire clonality and stability are correlated. FIG. 2311, the percent positive of various CD8, CD4 and B cell populations as a function of the cumulative frequency of segment G persistent clones. Color indicates DCB/No DCB.

FIG. 24A-24C depicts representative data showing Principal Component Analysis of peripheral TCR repertoire features, immuno-phenotyping and clinical laboratory measurements separated by patients' DCB status. FIG. 24A shows select clinical laboratory measurements (AST-SGOT, Creatinine and Hemoglobin concentration) from patients in each time-points. FIG. 24B shows Principal Component Analysis (PCA) of the joint peripheral measurements from the TCR repertoire, immuno-phenotyping and clinical measurements. FIG. 24C shows the fraction of clones in each patient which are shared with all 11 healthy donors (HD) versus the PC1 scores of those patients.

FIG. 24D represents an aggregated single matrix of principal component analysis (PCA) measurements taken at baseline from either the TCR repertoire analysis, the immunophenotyping of the PBMCs, or the clinical lab results. The matrix was centered and scaled, and PCA was calculated using the R function “prcomp” from the “stats” R package. The loadings, or contributions of the different measurements to PC1, were retrieved from the rotation matrix.

FIG. 25 depicts Kaplan-Meyer curves for progression free survival (PFS) of patients with PC1>0; versus patients with PC1<0.

FIG. 26 depicts representative data showing unique amino acids (left) and total TCR counts (right) of No DCB and DCB patients obtained from tumor samples collected at PreT=Pretreatment (Week 0 pre-Nivolumab).

FIG. 27 depicts a representative graph showing number of clones with shared unique amino acids as determined by a RNA sequencing clone detection from tumor samples and by iRepertoire from peripheral blood samples in the different non-overlapping (e.g., A, B, C) and overlapping (e.g., D, G, F) regions of a Venn diagram for peripheral blood TCR repertoires at the indicated time points, PreT=Pretreatment (Week 0 pre-Nivolumab); PreV=pre-vaccine administration; PostV=post-vaccine administration.

FIG. 28 depicts a representative data for tracked TCR clone frequency of clones shared with the tumor sample in DCB (left) and No DCB (right) patient peripheral samples at the indicated time points, PreT=Pretreatment (Week 0 pre-Nivolumab); PreV: pre-vaccine administration; PostV=post-vaccine administration.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

The use of the term “pretreatment” throughout refers to a patient sample collected at week 0 prior to the administration of Nivolumab and/or vaccine.

The present disclosure is based on important finding that the tumor microenvironment can be accurately assessed at a time point prior to, during and/or after a therapeutic treatment by evaluating a representative sample from the TME and evaluating a consolidated set of biomarkers which provide biomolecular signatures of the tumor condition. For the purpose of the disclosure, such biomolecular signatures constitute a TME signature. Moreover, in one aspect, the present disclosure identifies specific set of TME signatures, or at least one or more subsets of TME signatures from within a very complex tumor microenvironment, which is notoriously difficult in ascertaining reliable signal-to-noise ration because of the complexity; such that the specific set of TME signatures, or at least one or more subsets of TME signatures succinctly indicate the status of the tumor in relation to the one or more methods to which the TME signatures are thereafter applicable. The instant disclosure therefore embodies a breakthrough invention in relation to pretreatment, on-treatment or post-treatment assessment of durable clinical benefit for a therapy.

Also provided herein is highly predictive model developed based on the joint analysis of peripheral blood TCR repertoire features and the frequencies of T and B cell subpopulations at baseline. This prediction indicates an underlying susceptible immune state that is different between personalized neoantigen vaccine and anti-PD-1 treated patients who had a favorable response and those with poor response or healthy donors.

As used herein, the gene names used are well recognized to one of skill in eth art. In some cases, the gene name and the name of the protein encoded by the gene is used interchangeably within the application. As used herein, the gene names are collected from various sources and not pertaining to a single source of nomenclature. Irrespective of the deviation regarding gene nomenclature, one of skill in the art would be able to readily recognize the gene or genes referred to herein.

In some embodiments the TME signature comprises gene expression signature.

In some embodiments the TME signature comprises protein expression signature.

In some embodiments the TME signature comprises representative cells, the representative cellular composition, and/or a ratio or a proportion of cell types in the tumor.

In some embodiments the TME signature comprises expression of cell surface markers. Cell surface markers comprise Cluster of Differentiation proteins (CD) expressed on various cell types.

In some embodiments the TME signature comprises cytokines, chemokines, soluble proteins, glycoproteins, carbohydrates, or other biomolecules, including nucleic acids.

In some embodiments, TME comprises nucleic acids which are intracellular or extracellular, and comprise DNA, mRNA, hnRNA, dsRNA, ssRNA, miRNA, conjugated RNA or any other form of nucleic acid as known to one of skill in the art.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The terms “one or more” or “at least one,” such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and embodiments(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

The phrase “clonal composition characteristic” means the frequency distribution pattern of TCR clones which quantifies the dominance and/or diversity of a T cell repertoire. By way of example, this may include, but is not limited to Gini Coefficient, Shannon entropy, Diversity Evenness 50 (DE50), Sum of Squares, and Lorenz curve. The term “immune response” includes T cell mediated and/or B cell mediated immune responses that are influenced by modulation of T cell costimulation. Exemplary immune responses include T cell responses, e.g., cytokine production, and cellular cytotoxicity. In addition, the term “immune response” includes immune responses that are indirectly affected by T cell activation, e.g., antibody production (humoral responses) and activation of cytokine responsive cells, e.g., macrophages.

A “receptor” is to be understood as meaning a biological molecule or a molecule grouping capable of binding a ligand. A receptor can serve to transmit information in a cell, a cell formation or an organism. The receptor comprises at least one receptor unit and can contain two or more receptor units, where each receptor unit can consist of a protein molecule, e.g., a glycoprotein molecule. The receptor has a structure that complements the structure of a ligand and can complex the ligand as a binding partner. Signaling information can be transmitted by conformational changes of the receptor following binding with the ligand on the surface of a cell. According to the present disclosure, a receptor can refer to proteins of MHC classes I and II capable of forming a receptor/ligand complex with a ligand, e.g., a peptide or peptide fragment of suitable length.

A “ligand” is a molecule which is capable of forming a complex with a receptor. According to the present disclosure, a ligand is to be understood as meaning, for example, a peptide or peptide fragment which has a suitable length and suitable binding motives in its amino acid sequence, so that the peptide or peptide its amino acid sequence, so that the peptide or peptide fragment is capable of forming a complex with proteins of MHC class I or MHC class II.

An “antigen” is a molecule capable of stimulating an immune response, and can be produced by cancer cells or infectious agents or an autoimmune disease. Antigens recognized by T cells, whether helper T lymphocytes (T helper (TH) cells) or cytotoxic T lymphocytes (CTLs), are not recognized as intact proteins, but rather as small peptides that associate with class I or class II MHC proteins on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules. It is now understood that the peptides that associate with given class I or class II MHC molecules are characterized as having a common binding motif, and the binding motifs for a large number of different class I and II MHC molecules have been determined. Synthetic peptides that correspond to the amino acid sequence of a given antigen and that contain a binding motif for a given class I or II MHC molecule can also be synthesized. These peptides can then be added to appropriate APCs, and the APCs can be used to stimulate a T helper cell or CTL response either in vitro or in vivo. The binding motifs, methods for synthesizing the peptides, and methods for stimulating a T helper cell or CTL response are all known and readily available to one of ordinary skill in the art.

The term “peptide” is used interchangeably with “mutant peptide” and “neoantigenic peptide” in the present specification. Similarly, the term “polypeptide” is used interchangeably with “mutant polypeptide” and “neoantigenic polypeptide” in the present specification. By “neoantigen” or “neoepitope” is meant a class of tumor antigens or tumor epitopes which arises from tumor-specific mutations in expressed protein. The present disclosure further includes peptides that comprise tumor specific mutations, peptides that comprise known tumor specific mutations, and mutant polypeptides or fragments thereof identified by the method of the present disclosure. These peptides and polypeptides are referred to herein as “neoantigenic peptides” or “neoantigenic polypeptides.” The polypeptides or peptides can be a variety of lengths, either in their neutral (uncharged) forms or in forms which are salts, and either free of modifications such as glycosylation, side chain oxidation, phosphorylation, or any post-translational modification or containing these modifications, subject to the condition that the modification not destroy the biological activity of the polypeptides as herein described. In some embodiments, the neoantigenic peptides of the present disclosure can include: for MHC Class I, 22 residues or less in length, e.g., from about 8 to about 22 residues, from about 8 to about 15 residues, or 9 or 10 residues; for MHC Class II, 40 residues or less in length, e.g., from about 8 to about 40 residues in length, from about 8 to about 24 residues in length, from about 12 to about 19 residues, or from about 14 to about 18 residues. In some embodiments, a neoantigenic peptide or neoantigenic polypeptide comprises a neoepitope.

The term “epitope” includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three dimensional structural characteristics as well as specific charge characteristics.

A “T cell epitope” is a peptide sequence which can be bound by the MHC molecules of class I or II in the form of a peptide-presenting MHC molecule or MEW complex and then, in this form, be recognized and bound by cytotoxic T-lymphocytes or T-helper cells, respectively.

The term “antibody” as used herein includes IgG (including IgG1, IgG2, IgG3, and IgG4), IgA (including IgA1 and IgA2), IgD, IgE, IgM, and IgY, and is meant to include whole antibodies, including single-chain whole antibodies, and antigen-binding (Fab) fragments thereof. Antigen-binding antibody fragments include, but are not limited to, Fab, Fab′ and F(ab′)2, Fd (consisting of VH and CH1), single-chain variable fragment (scFv), single-chain antibodies, disulfide-linked variable fragment (dsFv) and fragments comprising either a VL or VH domain. The antibodies can be from any animal origin. Antigen-binding antibody fragments, including single-chain antibodies, can comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex. A person of skill in the art will recognize that a variety of immunoaffinity techniques are suitable to enrich soluble proteins, such as soluble HLA-peptide complexes or membrane bound HLA-associated polypeptides, e.g., which have been proteolytically cleaved from the membrane. These include techniques in which (1) one or more antibodies capable of specifically binding to the soluble protein are immobilized to a fixed or mobile substrate (e.g., plastic wells or resin, latex or paramagnetic beads), and (2) a solution containing the soluble protein from a biological sample is passed over the antibody coated substrate, allowing the soluble protein to bind to the antibodies. The substrate with the antibody and bound soluble protein is separated from the solution, and optionally the antibody and soluble protein are disassociated, for example by varying the pH and/or the ionic strength and/or ionic composition of the solution bathing the antibodies. Alternatively, immunoprecipitation techniques in which the antibody and soluble protein are combined and allowed to form macromolecular aggregates can be used. The macromolecular aggregates can be separated from the solution by size exclusion techniques or by centrifugation.

The term “immunopurification (IP)” (or immunoaffinity purification or immunoprecipitation) is a process well known in the art and is widely used for the isolation of a desired antigen from a sample. In general, the process involves contacting a sample containing a desired antigen with an affinity matrix comprising an antibody to the antigen covalently attached to a solid phase. The antigen in the sample becomes bound to the affinity matrix through an immunochemical bond. The affinity matrix is then washed to remove any unbound species. The antigen is removed from the affinity matrix by altering the chemical composition of a solution in contact with the affinity matrix.

The immunopurification can be conducted on a column containing the affinity matrix, in which case the solution is an eluent. Alternatively, the immunopurification can be in a batch process, in which case the affinity matrix is maintained as a suspension in the solution. An important step in the process is the removal of antigen from the matrix. This is commonly achieved by increasing the ionic strength of the solution in contact with the affinity matrix, for example, by the addition of an inorganic salt. An alteration of pH can also be effective to dissociate the immunochemical bond between antigen and the affinity matrix.

An “agent” is any small molecule chemical compound, antibody, nucleic acid molecule, or polypeptide, or fragments thereof.

An “alteration” or “change” is an increase or decrease. An alteration can be by as little as 1%, 2%, 3%, 4%, 5%, 10%, 20%, 30%, or by 40%, 50%, 60%, or even by as much as 70%, 75%, 80%, 90%, or 100%.

A “biologic sample” is any tissue, cell, fluid, or other material derived from an organism. As used herein, the term “sample” includes a biologic sample such as any tissue, cell, fluid, or other material derived from an organism. “Specifically binds” refers to a compound (e.g., peptide) that recognizes and binds a molecule (e.g., polypeptide), but does not substantially recognize and bind other molecules in a sample, for example, a biological sample.

“Capture reagent” refers to a reagent that specifically binds a molecule (e.g., a nucleic acid molecule or polypeptide) to select or isolate the molecule (e.g., a nucleic acid molecule or polypeptide).

As used herein, the terms “determining”, “assessing”, “assaying”, “measuring”, “detecting” and their grammatical equivalents refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

A “fragment” is a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

An “isolated” polypeptide (e.g., a peptide from a HLA-peptide complex) or polypeptide complex (e.g., a HLA-peptide complex) is a polypeptide or polypeptide complex of the present disclosure that has been separated from components that naturally accompany it. Typically, the polypeptide or polypeptide complex is isolated when it is at least 60%, by weight, free from the proteins and naturally-occurring organic molecules with which it is naturally associated. The preparation can be at least 75%, at least 90%, or at least 99%, by weight, a polypeptide or polypeptide complex of the present disclosure. An isolated polypeptide or polypeptide complex of the present disclosure can be obtained, for example, by extraction from a natural source, by expression of a recombinant nucleic acid encoding such a polypeptide or one or more components of a polypeptide complex, or by chemically synthesizing the polypeptide or one or more components of the polypeptide complex. Purity can be measured by any appropriate method, for example, column chromatography, polyacrylamide gel electrophoresis, or by HPLC analysis.

The term “vectors” refers to a nucleic acid molecule capable of transporting or mediating expression of a heterologous nucleic acid. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

### Tumor Microenvironment

The tumor microenvironment (TME) is complex. It is also a dynamic environment that changes as the tumor grows. It is one that supports the growth of a tumor and also the tumor suppressor factors are also readily found in such environment. The various characteristics of tumor include unlimited multiplication, evasion from growth suppressors, promoting invasion and metastasis, resisting apoptosis, stimulating angiogenesis, maintaining proliferative signaling, elimination of cell energy limitation, evading immune destruction, genome instability and mutation, and tumor enhanced inflammation. There are cellular and biomolecules associated with and assisting and/or resisting each of these functions, which makes the tumor microenvironment so complex. TME can support angiogenesis, tumor progression, and immune evasion from T lymphocyte recognition, as well as dictate response to cancer therapy. TME bears the signatures of the fate of the tumor. One of the main functions of the mammalian immune system is to monitor tissue homeostasis, to protect against invading or infectious pathogens and to eradicate damaged cells. Adaptive immune cells include thymus-dependent lymphocytes (T cells), and bursa-dependent lymphocytes (B cells). Innate immune cells consist of dendritic cells (DC), killer lymphocytes, natural killer (NK) cells, hyaline leukocyte/macrophage, granulocytes, and mast cells. Tumor cells express one or more mutated gene expression products, e.g., proteins or peptides, which are recognized by the body's immune system as foreign and are destroyed. Lymphocytes infiltrate the tumor to attack tumor cells and destroy. The interactions between the immune system and tumor include three phases: elimination, equilibrium and escape. During the elimination phase, immune cells of the innate and adaptive immune system recognize and destroy tumor cells. If the immune system cannot fully eliminate the tumor, the equilibrium phase occurs, during which tumor cells remain dormant and the immune system is not only sufficient to control tumor growth, but also shapes the immunogenicity of tumor cells.

In one embodiment, the presence of CD3⁺ tumor-infiltrating lymphocytes (TILs) was found to correlate with improved survival in epithelial ovarian cancer. Tumor infiltrating lymphocytes (TIL) interact most closely with the tumor cells and are likely to more accurately reflect tumor host interactions. Cytotoxic T cells, characterized as CD8+ T cells are important for attacking and killing tumor cells. In some occasions, CD4+ T cells take part in destroying tumor cells. In addition, there are NK cells, and γδT cells, which also are capable of killing tumor cells.

Tumor infiltration by a subpopulation of CD3⁺ CD4⁺ T cells with immunosuppressive properties (suppressor or regulatory T cells, Treg) can predict poor clinical outcome. Tumor has several immune evasion mechanisms, such as induction of tolerant T cells, Tregs and myeloid-derived suppressor cells (MDSCs) permit tumor growth. The primary mechanism of self-tolerance is central deletion in which self-reactive T cells are eliminated in the thymus by negative selection. Although most self-reactive cells are deleted by this mechanism, it is incomplete and additional tolerance mechanisms are required. The immune system has developed peripheral tolerance mechanisms to deal with self-reactive T cells in the periphery. Peripheral tolerance is regulated via different mechanisms that can be divided into those that regulate the responding state of T cells intrinsically (anergy, apoptosis and phenotype skewing) and those that provide extrinsic control (Tregs and tolerogenic dendritic cells [DCs]). Anergy was first shown in vitro as a result of T-cell receptor (TCR) ligation in the absence of costimulation. The common paradigm of T-cell activation describes the requirement of two signals to induce effector responses: MHC-peptide complexes (signal one) and costimulatory signal (signal two).

In some embodiments, the TME includes extracellular matrix signatures.

Although the specific examples described herein concern melanoma, the methods and compositions described herein are applicable to any other form of cancer or tumor including but not limited to liver cancer, ovarian cancer, cervical cancer, thyroid cancer, glioblastoma, glioma, leukemia, lymphoma, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, just as each type of tumor has specific immunological, pathophysiological and histological signatures that help in the identification and treatment of the disease, the specific state or condition at which a sample is analyzed from a tumor assists in determining the condition and fate of the tumor in a way that complements diagnostic and clinical decisions.

In some embodiments, the type of cells present in the tumor can provide a TME that can be related to a clinical outcome.

In some embodiments, the relative density of type of cells present in the tumor can provide a TME that can be related to a clinical outcome.

In some embodiments, the types of cells are measured by a gene expression analysis.

In some embodiments, the types of cells are measured by a protein expression analysis.

In some embodiments, the types of cells are measured by expression analysis of one or more proteins or peptides excreted or secreted in the extracellular milieu or presented on the cell surface.

In some embodiments, the types of cells are measured by relative expression of genes expressed in a first cell compared to genes expression in a second cell. In some embodiments, the abundance of one type of cell over another is measured.

In some embodiment, the type of cells are lymphocytes.

In some embodiment, the type of cells are T lymphocytes.

In some embodiment, the type of cells are CD8+ T lymphocytes.

In some embodiment, the types of cells are CD4+ T lymphocytes.

In some embodiment, the types of cells are memory lymphocytes.

In some embodiments, the type of cell are B lymphocytes.

In some embodiments, the types of cells are NK cells.

In some embodiments, the types of cells are non-immune cells.

In some embodiments, the types of cells are stromal cells.

In some embodiments, the types of cells are any combination of cells of the preceding types.

In some embodiments, a TME signature specific for a certain combination of cells is associated with a durable clinical benefit (DCB).

In some embodiments, DCB is determined to have been met if patient experiences at least a certain period of progression free survival (pfs) after treatment. In some embodiments, DCB is met with 36 weeks of pfs.

In some embodiments, an indicator of the activation status of the cell type is associated with DCB.

In some embodiments, an indicator of cellular interaction is associated with DCB.

In some embodiments, a TME signature comprising an indication of the presence of a certain cell type inside the tumor, or comprising an assessment of a ratio of or a proportion of a certain cell type with respect to another cell type in a tumor, and/or the activation state of the certain cell type, may provide indication of whether an intended therapy is likely to result in a favorable clinical outcome. A simplified exemplary situation could be as follows: a TME signature indicating high proportion of tumor infiltrating active cytotoxic cells, with low or absent Treg and other inhibitory cells, can indicate that an immunotherapy that involves cytotoxic T cells is likely to have clinical success on the tumor. In another exemplary situation: active MHCII signature can indicate that an immunotherapy relying on MHCII antigen presentation is likely to have clinical success on the tumor. However, although an investigation of a parameter of a tumor microenvironment as indicated in the exemplary situations above may indicate a certain feature or characteristic of a tumor, it should be appreciated by one of skill in the art that a random or non-systematic assessment of one or more such characteristics of a tumor in isolation, without further assessment of some other co-existing features of the tumor could be confounding for an assessment of the TME as such. Therefore, provided herein are carefully selected TME signatures, which constitute the biomarkers for the TME. Such biomarkers are intended for one or more purposes including, but not limited to: (a) a method of testing a patient having a cancer or a tumor for the presence or absence of an on-treatment biomarker for tumor microenvironment (TME) signatures that predict that the patient is likely to have an anti-tumor response to administering neoantigenic peptide vaccine; (b) a method for determining induction of tumor neoantigen specific T cells in a tumor; (c) a method of treating a patient having a tumor with a therapeutic regimen that comprises a first therapeutic agent if the TME biomarker is present; or treating the patient with a therapeutic regimen that does not include the first therapeutic agent if the TME biomarker is absent; (d) a method for testing a patient having a tumor for the presence or absence of a baseline biomarker that predicts that the patient is likely to have an anti-tumor response to a treatment with a therapeutic agent comprising neoantigens; (e) a kit for testing patients for the presence of absence of one or TME signature in a tumor sample.

### TME Signatures and Biomarkers

A biomarker, as used herein, is an indicator of a biological state or condition of the tumor, which can be measured. A TME signature can be used as a biomarker, provided the TME signature is indicative of a specific condition, either qualitatively, in which case, the signature is measured by the presence or absence of the signature, or quantitatively, in which case, the amount of or the degree of expression, increase or decrease compared to a suitable control.

In some embodiments, a TME signature is the expression of increase of or decrease of one or more biomolecules in the TME. In some embodiments, the TME is a signature of cell type(s) prevalent inside the tumor, the cytokines, chemokines or diffusible components secreted by the cell. According to the different clusters of differentiation, T cells are divided into CD4⁺ T (helper T cells, Th) and CD8⁺ T (cytotoxic T cells, Tc) cells. These secrete IFN-γ, TNF-α, and IL17, which have antitumor effects. B cells are mainly marked by different antigens in different physiological periods, such as mainly expressing CD19 and CD20 in pre-B cells, immature B cells, and plasma cells, mainly expressing IgM, IgD, and CR1 in mature B cells, and mainly expressing IgM, IgD, IgA, IgG in memory B cells. Human NK cells, which could efficiently recognize infected and malignant target cells, is the expression of HLA class I-specific receptors of the KIR and NKG2 gene families. DCs express co-stimulatory molecules and innate inflammatory cytokines, such as IL-12, IL-23, and IL-1, that promote IFN-γ-secreting CD4⁺ T cells and cytotoxic T lymphocyte responses. DCs represent key targets for 1,25-dihydroxyvitamin D3 (1,25(OH)₂D₃), which can directly induce T cells. CD28 and inducible costimulator (ICOS) are important costimulatory receptors required for T-cell activation and function, and deficiencies in both pathways lead to complete T-cell tolerance in vivo and in vitro. On the other hand, many negative costimulatory molecules that are either expressed by activated T cells, such as CTLA-4, PD-1 or APCs, tissue cells or tumor cells, such as PD-1 ligand 1, B7-S1 or B7-H3, have been discovered to regulate immune tolerance. Elevated expression of some of these molecules in the tumor microenvironment also suggests their participation in tumor evasion of immune surveillance and they may serve as potential targets for augmenting antitumor immunity. E3 ubiquitin ligases, including but not limited to Cbl-b, Itch and GRAIL, are components of the T-cell anergy. These molecules are clearly involved in the process of TCR downregulation, leading to the inability of T cells to produce cytokines and proliferate. In addition, transcriptional (transcriptional repressors) or even epigenetic (histone modification, DNA methylation and nucleosome positioning) mechanisms are involved to actively program tolerance through repressing cytokine gene transcription phenotype. Various tumor cells also express SPI-6 and SPI-CI, which cooperate to protect tumor cells from cytotoxicity. Furthermore, tumor cells do not usually express positive costimulatory molecules; by contrast, they express inhibitory receptors such as B7-H1 (PD-1 ligand), HLA-G, HLA-E and galectin-1. B7-H1 directly engages the inhibitory receptor PD-1 on tumor-specific CD4+ and CD8+ T cells; HLA-G interacts with the inhibitory receptor ILT2 on NK cells to impair their function; HLA-E binds to the inhibitory receptor CD94/NKG2A, and also the NK cell activating receptor CD94/NKG2C, both of which are mainly expressed by NK cells, and also by CD8+ T cells, and HLA-E also engages the TCR of CD8+ T cells, which inhibits their cytotoxic activity; and galectin-1 impairs TCR signaling of T cells, and also induces the generation of tolerogenic DCs, which promotes IL-10-mediated T-cell tolerance.

In some embodiments, therapy can result in aggregation of CD8⁺ and CD3⁺ T cells, and decrease of myeloid-derived suppressor cells and dendritic cells in the parental tumor, but not in the resistant tumors. CD4⁺ T cells and B cells may or may not change significantly. The CD8⁺ T cell infiltration after radiotherapy is important for tumor response, because in the nude mice and CD8⁺ T cell-depleted C57BL/6 mice, the parental and resistant tumor has similar radiosensitivity. Patients with good radiation response had more CD8⁺ T cells aggregation after radiotherapy. Radiotherapy resulted in robust transcription of T cell chemoattractant in the parental cells, and the expression of CCL5 was much higher.

In some embodiments, the disclosure contemplates human and non-human TME signatures, and uses thereof. Non-human (e.g., bovine, porcine, ovine, canine, feline) counterparts of the surface molecules, receptors, antigens, proteins or gene names or gene symbols of the human surface molecules, receptors, antigens, proteins or gene names or gene symbols described are easily available to one of skill in the art. Analogous methods of those methods described for human in the disclosure are applicable to non-human animals with the minimal required modifications known to one of the skill in the art.

In some embodiments, provided herein are TME signatures for durable clinical benefit (DCB). A DCB is a clinical outcome of a therapeutic treatment, where the patient is symptom free and/or disease free for a considerable period after the treatment, for as long as the rest of the patient's life.

In some embodiments, the TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, the B-cell signature comprises expression of a gene comprising CD19, CD20, CD21, CD22, CD24, CD27, CD38, CD40, CD72, CD3, CD79a, CD79b, IGKC, IGHD, MZB1, TNFRSF17, MS4A1, CD138, TNFRSR13B, GUSPB11, BAFFR, AID, IGHM, IGHE, IGHA1, IGHA2, IGHA3, IGHA4, BCL6, FCRLA or combinations thereof.

In some embodiments, the TLS signature comprises expression of a gene comprising CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, or combinations thereof.

In some embodiments, the TIS signature comprises CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT or a combination thereof.

In some embodiments, the effector/memory-like CD8+ T cell signature comprises expression of one or more genes encoding proteins comprising: CCR7, CD27, CD45RO, FLT3LG, GRAP2, IL16, IL7R, LTB, S1PR1, SELL, TCF7, CD62L, PLAC8, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PERI, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, or any combination thereof.

In some embodiments, the HLA-E/CD94 signature comprises expression of a gene CD94 (KLRD1), CD94 ligand, HLA-E, KLRC1 (NKG2A), KLRB1 (NKG2C) or any combination thereof.

In some embodiments, the HLA-E/CD94 signature further comprises an HLA-E:CD94 interaction level.

In some embodiments, the NK cell signature comprises expression of a gene CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, NCAM1, or a combination thereof.

In some embodiments, the MHC class II signature comprises expression of a gene that is an HLA comprising HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5 or a combination thereof.

In some embodiments, a biomarker for DCB comprises one component of a TME signature, e.g., a gene expression signature from the TLS signature.

In some embodiments, a biomarker for DCB comprises more than one component of a TME signature, wherein the TME signature is selected from a group consisting of: a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, or an MHC class II signature.

In some embodiments, a biomarker for DCB comprises one or more than one components of a first TME signature and at least one component of a second TME signature that is non-identical to the first TME signature, wherein the TME signatures are selected from a group consisting of: a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, a biomarker for DCB comprises one or more than one components of a first TME signature; one or more than one components of a second TME signature; and at least one component of a third TME signature; wherein the first, second and the third TME signatures are non-identical, wherein the TME signatures are selected from a group consisting of: a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, a biomarker for DCB comprises one or more than one components of a first TME signature; one or more than one components of a second TME signature; one or more than one components of a third TME signature; and at least one component of a fourth TME signature; wherein the first, the second, the third and the fourth TME signatures are non-identical, wherein the TME signatures are selected from a group consisting of: a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, a biomarker for DCB comprises one or more than one components of a first TME signature; one or more than one components of a second TME signature; one or more than one components of a third TME signature; and at least one component of a fourth TME signature; wherein the first, the second, the third and the fourth TME signatures are non-identical, wherein the TME signatures are selected from a group consisting of: a B-cell signature, a Tertiary Lymphoid Structures (TLS) signature, a Tumor Inflammation Signature (TIS), an effector/memory-like signature, an HLA-E/CD94 signature, a NK cell signature, and an MHC class II signature.

In some embodiments, a biomarker for DCB comprises one or more than one components of a first TME signature; one or more than one components of a second TME signature; one or more than one components of a third TME signature; one or more than one components of a fourth TME signature; and at least one component of a fifth TME signature; wherein the first, the second, the third, the fourth and the fifth TME signatures are non-identical, wherein the TME signatures are selected from a group consisting of: a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, a biomarker for DCB comprises one or more than one components of a first TME signature; one or more than one components of a second TME signature; one or more than one components of a third TME signature; one or more than one components of a fourth TME signature; and at least one component of a fifth TME signature; wherein the first, the second, the third, the fourth and the fifth TME signatures are non-identical.

In some embodiments, a biomarker for DCB comprises one or more than one components of a first TME signature; one or more than one components of a second TME signature; one or more than one components of a third TME signature; one or more than one components of a fourth TME signature; one or more than one components of a fifth TME signature; and at least one component of a sixth TME signature; wherein the first, the second, the third, the fourth, the fifth and the sixth TME signatures are non-identical.

In some embodiments, a biomarker for DCB comprises one or more than one components of a first TME signature; one or more than one components of a second TME signature; one or more than one components of a third TME signature; one or more than one components of a fourth TME signature; one or more than one components of a fifth TME signature; one or more than one components of a sixth TME signature; and at least one component of a seventh TME signature; wherein the first, the second, the third, the fourth, the fifth, the sixth and the seventh TME signatures are non-identical.

In some embodiments, a biomarker for DCB comprises a subset of TME signatures comprising a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, or an MHC class II signature.

In some embodiments, a biomarker for DCB comprises a subset of TME signatures comprising a gene expression signature from the TLS signature; and at least one component of another TME signature, e.g., a B cell signature.

In some embodiments, a biomarker for DCB comprises a subset of TME signatures comprising a gene expression signature from the TLS signature; and one or more components of another TME signature, e.g., a B cell signature, and/or a NK cell signature, and/or an MHC class II signature and/or an effector/memory-like CD8+ T cell signature and/or an HLA-E/CD94 signature.

In some embodiments, a higher normalized expression of a gene compared to a normalized baseline expression in the TME gene signature is associated with a positive biomarker classification for DCB where the therapy comprises neoantigen peptide therapy, comprising, one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein. In some embodiments, the method comprises a higher normalized gene expression of any one or more genes or genes encoding: CD19, CD20, CD21, CD22, CD24, CD27, CD38, CD40, CD72, CD3, CD79a, CD79b, IGKC, IGHD, MZB1, TNFRSF17, MS4A1, CD138, TNFRSR13B, GUSPB11, BAFFR, AID, IGHM, IGHE, IGHA1, IGHA2, IGHA3, IGHA4, BCL6, FCRLA CCR7, CD27, CD45RO, FLT3LG, GRAP2, IL16, IL7R, LTB, S1PR1, SELL, TCF7, CD62L, PLAC8, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PERI, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT, CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, NCAM1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, or HLA-DRB5 compared to a normalized baseline expression is associated with a positive biomarker classification for DCB with the therapeutic agent.

In some embodiments, a lower normalized expression of a gene compared to a normalized baseline expression in the TME gene signature is associated with a positive biomarker classification for DCB where the therapy comprises neoantigen peptide therapy, comprising, a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein. In some embodiments, a lower normalized expression of B7-H3 expression compared to baseline expression levels, is associated with a positive biomarker for DCB.

In some embodiments a biomarker for TME comprises one or more signatures that are higher than a baseline value, and one or more signatures that are lower than a baseline value.

In some embodiments, the baseline level of the TME signature is the state of the same component in the signature (e.g. gene expression level, protein level, peptide level, protein interaction level, or protein activity level) in the patient or the subject before the treatment in question was administered.

In some embodiments, the baseline level of the TME signature is a comparison of the patient's signature of the same component in the signature (e.g. gene expression level, protein level, peptide level, protein interaction level, or protein activity level) in a comparable non-tumor tissue.

In some embodiments, the baseline level of the TME signature is a comparison with a patient's signature of the same component in the signature (e.g. gene expression level, protein level, peptide level, protein interaction level, or protein activity level) in a control subject, or an universal control, e.g. control created from a collection of control subjects, or archived data.

In some embodiments, the TME signature is calculated as a weighted average of the log 2 expression levels of all the genes or gene products which have been taken into consideration, after first being normalized to an internal constant (such as, a set of housekeeping gene expressions). In an exemplary gene expression analysis, for a TME signature biomarker for each sample of n gene names: having G₁, G₂, . . . , Gₙ and m housekeeping genes Hk₁, Hk₂, . . . , Hkₘ, an exemplary weighted average gene signature calculation is:

$$( {w}_{1} {g}_{1}^{′} + {w}_{2} {g}_{2}^{′} + … + {w}_{n} {g}_{n}^{′} ) \text{/} ( {w}_{1} + {w}_{2} + … + {w}_{n} )$$

where w₁, w₂, . . . , wₙ are weights of each gene G₁, G₂, . . . , Gₙ; wherein each of g₁′, g₂′, . . . , gₙ′ are the log 2 normalized gene expression analysis of gene G₁, G₂, . . . , Gₙ and, g₁′ can be calculated as:

$$Log 2 [ {g}_{1} \text{/} ( {hk}_{1} + {hk}_{2} + … + {hk}_{m 2} ) \text{/} m ] + 10 - Log 2 [ ( {hk}_{1} + {hk}_{2} + … + {hk}_{m} ) \text{/} m ] ,$$

where g₁, g₂, . . . , gₙ are the gene expressions of the genes G₁, G₂, . . . , Gₘ; hk₁, hk₂, . . . , hkₘ are the gene expressions of the housekeeping genes Hk₁, Hk₂, . . . , Hkₘ, and 10−Log 2[(hk₁+hk₂+ . . . +hkₘ)/m] is a Factor that brings the housekeeping gene expressions to the same level across all samples to address input sample variation.

In some embodiments the TME signature biomarker is a weighted average gene signature of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 genes.

In some embodiments the TME signature biomarker is a weighted average gene signature of 31, 32, 33, 34, 35, 36, 37, 38, 39 40, 41, 42, 43, 44, 45, 46, 47, 48, 49, 50 genes.

In some embodiments the TME signature biomarker is a weighted average gene signature of 51, 52, 53, 54, 55, 56, 57, 58, 59, 60, 61, 62, 63, 64, 65, 66, 67, 68, 69, 70 genes.

In some embodiments the TME signature biomarker is a weighted gene signature of 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 genes.

In some embodiments, the normalized expression of one or more genes compared to baseline is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher.

In some embodiments, the normalized expression of one or more genes compared to baseline is at least 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold higher.

In some embodiments, the normalized expression of one or more genes compared to baseline is at least 55-fold, 60-fold, 65-fold, 70-fold, 75-fold, 80-fold, 85-fold, 90-fold, 95-fold, 100-fold higher or higher by any fold change within.

In some embodiments, the normalized expression of one or more genes compared to baseline is at least 200-fold, 300-fold, 400-fold, 500-fold, 600-fold, 700-fold, 800-fold 1000-fold or 10,000 fold higher or higher by any fold change within.

In some embodiments, the normalized expression of one or more genes compared to baseline is at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower.

In some embodiments, the normalized expression of one or more genes compared to baseline is at least 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold lower.

In some embodiments, the normalized expression of one or more genes compared to baseline is at least 55-fold, 60-fold, 65-fold, 70-fold, 75-fold, 80-fold, 85-fold, 90-fold, 95-fold, 100-fold lower or lower by any fold change within.

In some embodiments, the normalized expression of one or more genes compared to baseline is at least 200-fold, 300-fold, 400-fold, 500-fold, 600-fold, 700-fold, 800-fold 1000-fold or 10,000 fold lower or lower by any fold change within.

In some embodiments, the presence of a TME signature in a subject with cancer indicates that the subject is more likely to receive durable clinical benefit from a treatment than a subject with the cancer that does not have the TME signature. For example, the presence of a 2A6 or more functional Ig CDR3s (e.g., as observed by RNA-seq) from cells of a TME sample from a subject with cancer can indicate the subject is likely to receive durable clinical benefit from a treatment. For example, the presence of a 2\hat{ }7, 2\hat{ }8, 2\hat{ }9, 2\hat{ }0, 2\hat{ }11 or 2\hat{ }12 or more functional Ig CDR3s (e.g., as observed by RNA-seq) from cells of a TME sample from a subject with cancer can indicate the subject is likely to receive durable clinical benefit from a treatment.

### Peripheral Blood Signatures

Contemplated herein are some peripheral blood biomarkers in a subject with cancer, which can be used in one of the following ways: (i) presence or absence of a marker can indicate any one or more of the nature, state of progression or responsiveness of the disease to a drug or therapy; (2) presence or absence of a marker can indicate whether the subject can be responsive to a drug or therapy; (3) presence or absence of a marker can indicate whether the outcome of the treatment with a drug or a therapy will be favorable or not; (4) presence or absence of a marker can be used to determine the dose, frequency, regimen of a drug or a therapy. The peripheral blood biomarkers can be detected in a subject before the onset of a therapy. The peripheral blood biomarkers can be detected in a subject during a therapy. The peripheral blood biomarkers can be detected in a subject as a consequence of a therapy. Exemplary peripheral biomarkers are provided herein.

In some embodiments, the presence of a peripheral blood signature in a subject with cancer indicates that the subject is more likely to receive durable clinical benefit from a treatment than a subject with the cancer that does not have the peripheral blood signature.

For example, the presence of a naïve T cell population of 20% or less of total CD8+ T cells in a peripheral blood sample from a subject with cancer can indicate the subject is likely to receive durable clinical benefit from a treatment. For example, the presence of a naïve T cell population of 19%, 18%, 17%, 16%, 15%, 14%, 13%, 12%, 11%, 10%, 9%, 8%, 7%, 6%, 5%, 4%, 3%, or 2% or less of total CD8+ T cells in a peripheral blood sample from a subject with cancer can indicate the subject is likely to receive durable clinical benefit from a treatment.

For example, the presence of an effector memory T cell population of 40% or greater of total CD8+ T cells in a peripheral blood sample from a subject with cancer can indicate the subject is likely to receive durable clinical benefit from a treatment. For example, the presence of an effector memory T cell population of 45%, 50%, 55%, 60%, 65%, 70%, 75%, 80%, 85%, 90%, or 95% or greater of total CD8+ T cells in a peripheral blood sample from a subject with cancer can indicate the subject is likely to receive durable clinical benefit from a treatment.

For example, the presence of a naïve B cell population of 70% or less of total CD19+ B cells in a peripheral blood sample from a subject with cancer can indicate the subject is likely to receive durable clinical benefit from a treatment. For example, the presence of a naïve B cell population of 65%, 60%, 55%, 50%, 45%, 40%, 35%, 30%, 25%, 20%, 15%, 10% or 5% or less of total CD19+ B cells in a peripheral blood sample from a subject with cancer can indicate the subject is likely to receive durable clinical benefit from a treatment.

For example, the presence of a class-switched memory B cell population of greater than 10% of total CD19+ B cells in a peripheral blood sample from a subject with cancer can indicate the subject is likely to receive durable clinical benefit from a treatment. For example, the presence of a class-switched memory B cell population of greater than 15%, 20%, 25%, 30%, 35%, 40%, 45%, 50%, 55%, 60%, or 65% of total CD19+ B cells in a peripheral blood sample from a subject with cancer can indicate the subject is likely to receive durable clinical benefit from a treatment.

For example, the presence of a plasmacytoid DC population of 3% or less of total Lin−/CD11c− cells in a peripheral blood sample from a subject with cancer can indicate the subject is likely to receive durable clinical benefit from a treatment. For example, the presence of a plasmacytoid DC population of 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, or 0.2% or less of total Lin−/CD11c− cells in a peripheral blood sample from a subject with cancer can indicate the subject is likely to receive durable clinical benefit from a treatment.

For example, the presence of a CTLA4+ CD4 T cell population of 9% or less of total CD4+ T cells in a peripheral blood sample from a subject with cancer can indicate the subject is likely to receive durable clinical benefit from a treatment. For example, the presence of a CTLA4+ CD4 T cell population of 8%, 7%, 6%, 5%, 4%, 3%, 2% or 1% or less of total CD4+ T cells in a peripheral blood sample from a subject with cancer can indicate the subject is likely to receive durable clinical benefit from a treatment.

For example, the presence of a memory CD8+ T cells population of 40% or more or 55% or more of total CD8+ T cells in a peripheral blood sample from a subject with cancer at a post-vaccine time point can indicate the subject is likely to receive durable clinical benefit from a treatment. For example, the presence of a memory CD8+ T cells population of 45%, 50%, 55%, 60%, 65%, 70%, 75%, 80%, 85%, 90%, or 95% or more of total CD8+ T cells in a peripheral blood sample from a subject with cancer at a post-vaccine time point can indicate the subject is likely to receive durable clinical benefit from a treatment.

### Peripheral Blood Mononuclear Cells

Contemplated herein are signatures within the peripheral blood mononuclear cells, that can be analyzed by cytometry and immunohistochemistry, among other methods. Peripheral blood mononuclear cells is isolated from a subject prior to treatment and is subjected to analysis for proportions of individual cell types, expression of one or more specific cell surface molecules, one or more specific cytoplasmic or nuclear molecules, and degree of such expression. Similar analysis is performed in subjects under ongoing treatment and/or subjects who have completed a therapeutic regiment. A correlation can then be sought between the analyzed parameters and clinical outcome of the therapy. In summary, analysis of such parameters in completed and ongoing clinical studies can identify potential associations of certain parameters or characteristics with a durable clinical benefit. A positive association of a parameter with DCB can help generate a signature for DCB at pretreatment, such that presence of a certain parameter within the PBMCs at the time of analysis prior to a subject being administered a therapy, may be used to predict an outcome for the therapy, whether or not DCB may be met.

A large number of parameters are considered for potential peripheral blood signatures of DCB. These include but are not limited to: CD4:CD8 T cell ratio, proportions of memory T cells and naïve CD4 and CD8 T cell subsets, proportion of T regulatory cells, T cell PD1 expression, T cell CTLA-4 expression, proportions of gamma-delta T cells, proportions of myeloid cells, proportions of monocytes, proportions of CD11c+ DCs, CD141+ CLEC9A+DCs, proportions of plasmacytoid DCs, proportions of NK cells (including activation/inhibitory receptor expression and Perforin/Granzyme B expression), proportions of B cells. The signatures can be used as an inclusion or exclusion criteria for future patient enrollment, and/or characterize a patient's molecular response over the course of treatment.

### Apolipoprotein E

Apolipoprotein E (ApoE) is a secreted protein and plays a major role in the metabolism of cholesterol and triglycerides by acting as a receptor-binding ligand mediating the clearance of chylomicrons and very-low density cholesterol from plasma. The ApoE gene on chromosome 19 (APOE locus 19q13.3.1) has three common alleles (E2, E3, E4), which encode three major ApoE isoforms, leading to ApoE2, ApoE3 and ApoE4 protein isoform products respectively. The haplotypes result from combination of the alleles of the two single nucleotide polymorphisms rs429358 and rs7412. The isoforms differ site residues 112 and 158 (see Table 1 below).

TABLE 1

| | ApoE2 | ApoE3 | ApoE4 |
|---|---|---|---|
| Protein | R158C | Reference | C112R |
| substitution | | | |
| Genome change | chr19: 44908822(C > T) | Reference | chr19: 44908684(T > C) |
| (UCSC hg38 | | | |
| coordinates) | | | |
| SNP ID | rs7412 | Reference | rs429358 |
| Associations | type III | | Alzheimers |
| | hyperlipoproteinemia | | |
| Worldwide allele | 8.4% | 77.9% | 13.7% |
| frequencies | | | |
| Biology | Binds poorly to cell | “neutral” | Preferential binding to |
| | surface receptors | | VLDL (as opposed to |
| | | | HDL) |

Consequently, a subject may be homozygous or heterozygous for E2, E3 and E4. Carriers of the e2 allele have defective receptor-binding ability and lower circulating cholesterol levels and higher triglyceride levels, while carriers of the e4 allele appear to have higher plasma levels of cholesterol. A recent meta-analysis of ApoE genotypes and coronary heart disease (CHD) showed that people with the e4 allele had a 42% greater risk of CHD than those with the e3/e3 genotype. Germline variant ApoE4 is associated with Alzheimer's disease. In some embodiments, a subject with e4 allele may have reduced NMDA or AMPA receptor functions. In some embodiments, a subject with e4 allele may have higher intracellular calcium levels in neuronal cells. In some embodiments, a subject with e4 allele may have an altered calcium response to NMDA in neuronal cells. In some embodiments, a subject with e4 allele may have impaired glutamatergic neurotransmission. In some embodiments, a subject with e4 allele may have higher serum vitamin D levels than a subject with ApoE2 or ApoE3. In some embodiments, a subject with e4 allele may have an enhanced Aβ oligomerization, and is predisposed to Alzheimer's disease.

Variants of ApoE have been associated with lipid and triglyceride levels and influence insulin sensitivity. In some embodiments, a subject with e2 allele has higher cholesterol efflux from cells compared to a subject with e3 or e4 allele. Carriers of e2 allele may have lower total cholesterol (TC), lower LDL and higher levels of HDL compared to a subject with e3/e3 homozygous alleles. In some embodiments, the carrier of an e2 allele may have lower risk of coronary heart disease (CHD). In some embodiments, carriers of e4 alleles have higher TC, higher LDL, lower HDL, and may be at a higher risk for CHD compared to a subject with e3/e3 alleles.

ApoE variants are associated with risk of inflammation. In some embodiments, a subject having an e4 allele may have smaller APOE lipoproteins and lower APOE levels in the cerebrospinal fluid (CSF), plasma or interstitial fluid.

The present invention leads to a method of treatment of a disease in a subject, e.g. cancer, the method comprising a step of determining whether or not the subject has one or more genetic variations of ApoE allele, comprising (i) an ApoE2 allele, or an ApoE4 allele.

In some embodiments, the subject is heterozygous for E2 allele. In some embodiments, the subject is heterozygous for E4 allele. In some embodiments, the subject is heterozygous for E3 allele. In some embodiments the subject is homozygous for E2 allele. In some embodiments the subject is homozygous for E4 allele. In some embodiments the subject is homozygous for E3 allele.

In some embodiments the subject comprises an ApoE genetic variation comprising (i) an ApoE2 genetic variation comprising a sequence encoding a R158C ApoE protein or (ii) an ApoE4 genetic variation comprising a sequence encoding a C112R ApoE protein. In some embodiments, subject comprises an ApoE3 allele comprising a sequence encoding an ApoE protein that does not include R158C or C112R ApoE protein sequence variants. In some embodiments the subject has rs7412-T and rs429358-T. In some embodiment, the subject has rs7412-C and rs429358-C. In some embodiments, the one or more genetic variations comprises chr19:44908684 T>C; wherein chromosome positions of the one or more genetic variations are defined with respect to UCSC hg38. In some embodiments, the one or more genetic variations comprises chr19:44908822 C>T; wherein chromosome positions of the one or more genetic variations are defined with respect to UCSC hg38.

In some embodiments, a reference is a subject who homozygous for the ApoE3 allele. In some embodiments, a reference subject that is homozygous for the ApoE3 allele has a decreased likelihood of responding to the cancer therapeutic agent.

In some embodiments, the cancer therapeutic agent comprises (i) one or more peptides comprising a cancer epitope of a protein, (ii) a polynucleotide encoding the one or more peptides, (iii) one or more APCs comprising the one or more peptides or the polynucleotide encoding the one or more peptides, or (iv) a T cell receptor (TCR) specific for a cancer epitope of the one or more peptides in complex with an HLA protein.

In some embodiments the cancer is melanoma. In some embodiments, the cancer therapeutic agent comprises an immunomodulatory agent. In some embodiments, the cancer therapeutic agent comprises an anti-PD1 agent or an anti-PD1 antibody.

In some embodiments the cancer is melanoma.

In some embodiments the cancer is lung cancer.

In some embodiments the cancer is bladder cancer.

In some embodiments the cancer is colon cancer.

In some embodiments, the cancer is liver cancer.

In some embodiments, identification of an ApoE genetic variant that is not the reference haplotype indicates the likelihood that the subject will not respond favorably to the peptide therapy and/or anti-PD1 therapy, or a combination of the peptide and anti-PD1 therapy. In some embodiments, the likelihood of decreased response can be 1%-5%, 0.1%-10%, 5%-20% 2%-30% 10%-30%, 5%-50%, 10%-50% or 10%-60%, or 2%-80%, or 1%-90% of the expected outcome in the subject with reference haplotype, where the response is measured by tumor regression at a certain time period in response to the therapy.

### Compositions and Methods of Treatment

### Neoantigen

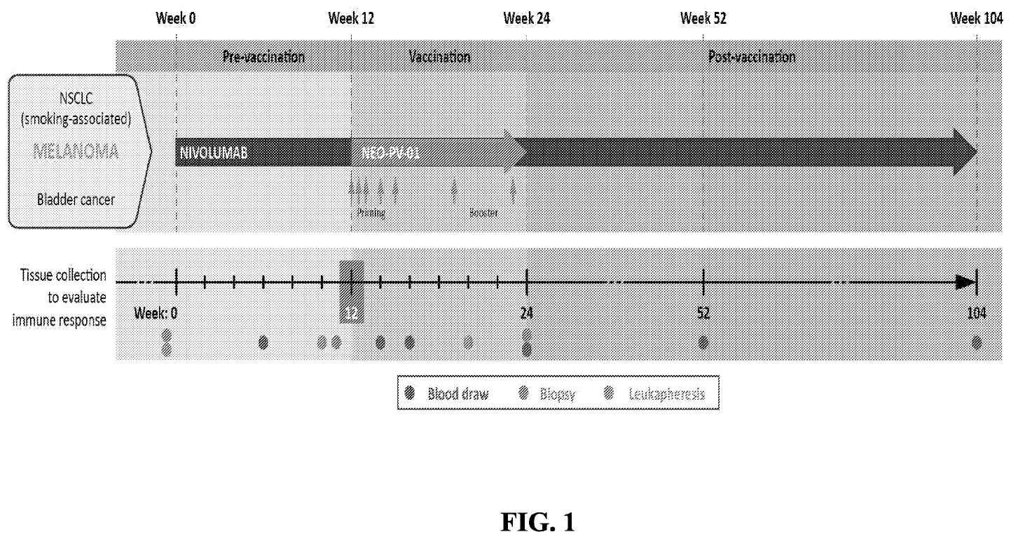

Neoantigens arise from DNA mutations and are critical targets that are presented on the surface of cancer cells for tumor-specific T cell responses. Vaccines targeting neoantigens have the potential to induce de novo and amplify pre-existing anti-tumor T cell responses. NEO-PV-01 is a personal neoantigen vaccine custom-designed and manufactured specifically for the mutational profile of each individual's tumor (FIG. 1). Neoantigens are isolated neoantigenic peptide comprising a tumor-specific neoepitope, wherein the isolated neoantigenic peptide is not a native polypeptide, wherein the neoepitope comprises at least 8 contiguous amino acids of an amino acid sequence represented by: AxByCz wherein each A is an amino acid corresponding to a first native polypeptide; each B is an amino acid that is not an amino acid corresponding to the first native polypeptide or the second native polypeptide, each C is an amino acid encoded by a frameshift of a sequence encoding a second native polypeptide; x+y+z is at least 8, wherein y is absent and the at least 8 contiguous amino acids comprises at least one Cz, or y is at least 1 and the at least 8 contiguous amino acids comprises at least one By and/or at least one Cz.

In some embodiments, the neoantigen is delivered as an isolated polynucleotide encoding an isolated neoantigenic peptide described herein. In some embodiments, the polynucleotide is DNA. In some embodiments, the polynucleotide is RNA. In some embodiments, the RNA is a self-amplifying RNA. In some embodiments, the RNA is modified to increase stability, increase cellular targeting, increase translation efficiency, adjuvanticity, cytosol accessibility, and/or decrease cytotoxicity. In some embodiments, the modification is conjugation to a carrier protein, conjugation to a ligand, conjugation to an antibody, codon optimization, increased GC-content, incorporation of modified nucleosides, incorporation of 5′-cap or cap analog, and/or incorporation of an unmasked poly-A sequence. In some embodiments, the neoantigen is delivered as a cell comprising the polynucleotide described herein. In some embodiments the neoantigen is delivered in is a vector comprising the polynucleotide described herein. In some embodiments, the polynucleotide is operably linked to a promoter. In some embodiments, the vector is a self-amplifying RNA replicon, plasmid, phage, transposon, cosmid, virus, or virion. In some embodiments, the vector is derived from an adeno-associated virus, herpesvirus, lentivirus, or a pseudotype thereof. Provided herein is an in vivo delivery system comprising the isolated polynucleotide described herein.

In some embodiments, the delivery system includes spherical nucleic acids, viruses, virus-like particles, plasmids, bacterial plasmids, or nanoparticles.

In some embodiments, the cell is an antigen presenting cell. In some embodiments, the cell is a dendritic cell. In some embodiments, the cell is an immature dendritic cell.

In some embodiments, at least one of the additional neoantigenic peptide is specific for an individual subject's tumor. In some embodiments, the subject specific neoantigenic peptide is selected by identifying sequence differences between the genome, exome, and/or transcriptome of the subject's tumor sample and the genome, exome, and/or transcriptome of a non-tumor sample. In some embodiments, the samples are fresh or formalin-fixed paraffin embedded tumor tissues, freshly isolated cells, or circulating tumor cells. In some embodiments, the sequence differences are determined by Next Generation Sequencing.

In some embodiments, a neoantigenic peptide that is delivered is characterized by high affinity binding to a specific HLA peptide, which HLA peptide is found in the recipient it is delivered to. In some embodiments, the peptide is delivered in addition to a T cell receptor (TCR) capable of binding at least one neoantigenic peptide described herein or an MHC-peptide complex comprising at least one neoantigenic peptide is described herein. The TCR may be comprised in a vector, a vector capable of being expressed in a cell.

In some embodiments, the neoepitope of a protein are selected from a group of peptides predicted by a HLA binding predictive platform, wherein the HLA binding predictive platform is a computer based program with a machine learning algorithm, and where in the machine learning algorithm integrates a multitude of information related to a peptide and a human leukocyte antigen to which it associates, comprising peptide amino acid sequence information, structural information, association and or dissociation kinetics information and mass spectrometry information.

In some embodiments, the MHC of the MHC-peptide is MHC class I or class II. In some embodiments, the TCR is a bispecific TCR further comprising a domain comprising an antibody or antibody fragment capable of binding an antigen. In some embodiments, the antigen is a T cell-specific antigen. In some embodiments, the antigen is CD3. In some embodiments, the antibody or antibody fragment is an anti-CD3 scFv. In some embodiments, the receptor is a chimeric antigen receptor comprising: (i) a T cell activation molecule; (ii) a transmembrane region; and (iii) an antigen recognition moiety capable of binding at least one neoantigenic peptide described herein or an MHC-peptide complex comprising at least one neoantigenic peptide described herein. In some embodiments, CD3− zeta is the T cell activation molecule. In some embodiments, the chimeric antigen receptor further comprises at least one costimulatory signaling domain. In some embodiments, the signaling domain is CD28, 4-1BB, ICOS, OX40, ITAM, or Fc epsilon RI-gamma. In some embodiments, the antigen recognition moiety is capable of binding the isolated neoantigenic peptide in the context of MHC class I or class II. In some embodiments, the chimeric antigen receptor comprises the CD3− zeta, CD28, CTLA-4, ICOS, BTLA, KIR, LAG3, CD137, OX40, CD27, CD40L, Tim-3, A2aR, or PD-1 transmembrane region. In some embodiments, the neoantigenic peptide is located in the extracellular domain of a tumor associated polypeptide. In some embodiments, the MEW of the MHC-peptide is MEW class I or class II.

In some embodiments, the immunotherapy comprises a T cell comprising a T cell receptor (TCR) capable of binding at least one neoantigenic peptide described herein or an MHC-peptide complex comprising at least one neoantigenic peptide described herein, wherein the T cell is a T cell isolated from a population of T cells from a subject that has been incubated with antigen presenting cells and one or more of the at least one neoantigenic peptide described herein for a sufficient time to activate the T cells. In some embodiments, the T cell is a CD8+ T cell, a helper T cell or cytotoxic T cell.

In some embodiments, the population of T cells from a subject is a population of CD8+ T cells from the subject. In some embodiments, the one or more of the at least one neoantigenic peptide described herein is a subject-specific neoantigenic peptide. In some embodiments, the subject-specific neoantigenic peptide has a different tumor neo-epitope that is an epitope specific to a tumor of the subject. In some embodiments, the subject-specific neoantigenic peptide is an expression product of a tumor-specific non-silent mutation that is not present in a non-tumor sample of the subject. In some embodiments, the subject-specific neoantigenic peptide binds to an HLA protein of the subject. In some embodiments, the subject-specific neoantigenic peptide binds to a HLA protein of the subject with an IC50 less than 500 nM. In some embodiments, the activated CD8+ T cells are separated from the antigen presenting cells.

In some embodiments, the antigen presenting cells are dendritic cells or CD40L− expanded B cells. In some embodiments, the antigen presenting cells are non-transformed cells. In some embodiments, the antigen presenting cells are non-infected cells. In some embodiments, the antigen presenting cells are autologous. In some embodiments, the antigen presenting cells have been treated to strip endogenous MHC-associated peptides from their surface. In some embodiments, the treatment to strip the endogenous MHC-associated peptides comprises culturing the cells at about 26° C. In some embodiments, the treatment to strip the endogenous MHC-associated peptides comprises treating the cells with a mild acid solution. In some embodiments, the antigen presenting cells have been pulsed with at least one neoantigenic peptide described herein. In some embodiments, pulsing comprises incubating the antigen presenting cells in the presence of at least about 2 μg/ml of each of the at least one neoantigenic peptide described herein. In some embodiments, ratio of isolated T cells to antigen presenting cells is between about 30:1 and 300:1. In some embodiments, the incubating the isolated population of T cells is in the presence of IL-2 and IL-7. In some embodiments, the MEW of the MHC-peptide is MHC class I or class II.

### Treatment Methods

In one embodiment, a method of treating cancer or initiating, enhancing, or prolonging an anti-tumor response in a subject in need thereof comprises administering to the subject the peptide, polynucleotide, vector, composition, antibody, or cells described herein. In some embodiments, the subject is a human. In some embodiments, the subject has cancer. In some embodiments, the cancer is selected from the group consisting of urogenital, gynecological, lung, gastrointestinal, head and neck cancer, malignant glioblastoma, malignanmesothelioma, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer. In some embodiments, the peptide, polynucleotide, vector, composition, antibody, or cells described herein is for use in treating a subject with an HLA type that is a corresponding HLA type. In some embodiments, the subject has undergone surgical removal of the tumor. In some embodiments, the peptide, polynucleotide, vector, composition, or cells is administered via intravenous, intraperitoneal, intratumoral, intradermal, or subcutaneous administration. In some embodiments, the peptide, polynucleotide, vector, composition, or cells is administered into an anatomic site that drains into a lymph node basin. In some embodiments, administration is into multiple lymph node basins. In some embodiments, administration is by a subcutaneous or intradermal route. In some embodiments, peptide is administered. In some embodiments, administration is intratumorally. In some embodiments, polynucleotide, optionally RNA, is administered. In some embodiments, the polynucleotide is administered intravenously. In some embodiments, the cell is a T cell or dendritic cell. In some embodiments, the peptide or polynucleotide comprises an antigen presenting cell targeting moiety. In some embodiments, the cell is an autologous cell. In some embodiments, the method further comprises administering at least one immune checkpoint inhibitor to the subject. In some embodiments, the checkpoint inhibitor is a biologic therapeutic or a small molecule. In some embodiments, the checkpoint inhibitor is selected from the group consisting of a monoclonal antibody, a humanized antibody, a fully human antibody and a fusion protein or a combination thereof. In some embodiments, the checkpoint inhibitor is a PD-1 antibody or a PD-L1 antibody. In some embodiments, the checkpoint inhibitor is selected from the group consisting of ipilimumab, tremelimumab, nivolumab, avelumab, durvalumab, atezolizumab, pembrolizumab, and any combination thereof. In some embodiments, the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands, and any combination thereof. In some embodiments, the checkpoint inhibitor interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In some embodiments, two or more checkpoint inhibitors are administered. In some embodiments, at least one of the two or more checkpoint inhibitors is a PD-1 antibody or a PD-L1 antibody. In some embodiments, at least one of the two or more checkpoint inhibitors is selected from the group consisting of ipilimumab, tremelimumab, nivolumab, avelumab, durvalumab, atezolizumab, and pembrolizumab. In some embodiments, the checkpoint inhibitor and the composition are administered simultaneously or sequentially in any order. In some embodiments, the peptide, polynucleotide, vector, composition, or cells is administered prior to the checkpoint inhibitor. In some embodiments, the peptide, polynucleotide, vector, composition, or cells is administered after the checkpoint inhibitor. In some embodiments, administration of the checkpoint inhibitor is continued throughout neoantigen peptide, polynucleotide, vector, composition, or cell therapy. In some embodiments, the neoantigen peptide, polynucleotide, vector, composition, or cell therapy is administered to subjects that only partially respond or do not respond to checkpoint inhibitor therapy. In some embodiments, the composition is administered intravenously or subcutaneously. In some embodiments, the checkpoint inhibitor is administered intravenously or subcutaneously. In some embodiments, the checkpoint inhibitor is administered subcutaneously within about 2 cm of the site of administration of the composition. In some embodiments, the composition is administered into the same draining lymph node as the checkpoint inhibitor. In some embodiments, the method further comprises administering an additional therapeutic agent to the subject either prior to, simultaneously with, or after treatment with the peptide, polynucleotide, vector, composition, or cells. In some embodiments, the additional agent is a chemotherapeutic agent, an immunomodulatory drug, an immune metabolism modifying drug, a targeted therapy, radiation an anti-angiogenesis agent, or an agent that reduces immune-suppression. In some embodiments, the chemotherapeutic agent is an alkylating agent, a topoisomerase inhibitor, an anti-metabolite, or an anti-mitotic agent. In some embodiments, the additional agent is an anti-glucocorticoid induced tumor necrosis factor family receptor (GITR) agonistic antibody or antibody fragment, ibrutinib, docetaxeol, cisplatin, a CD40 agonistic antibody or antibody fragment, an DO inhibitor, or cyclophosphamide. In some embodiments, the method elicits a CD4+ T cell immune response or a CD8+ T cell immune response. In some embodiments, the method elicits a CD4+ T cell immune response and a CD8+ T cell immune response.

In one aspect, provided herein is a method of treating a patient having a tumor comprising: (I) determining if a sample collected from the patient is positive or negative for a biomarker which predicts that the patient is likely to have an anti-tumor response to a first therapeutic agent comprising (i) a one or more peptides comprising a neoepitope of a protein, (ii) a polynucleotide encoding the one or more peptides, (iii) one or more APCs comprising the one or more peptides or the polynucleotide encoding the one or more peptides, or (iv) a T cell receptor (TCR) specific for a neoepitope of the one or more peptides in complex with an HLA protein, and (II) treating the patient with a therapeutic regimen that comprises the first therapeutic agent if the biomarker is present; or treating the patient with a therapeutic regimen that does not include the first therapeutic agent if the biomarker is absent, wherein the biomarker comprises a tumor microenvironment (TME) signature. The TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

In some embodiments, provided herein is a method of treating a patient having a tumor comprising: (I) determining if a sample collected from the patient is positive or negative for a biomarker which predicts that the patient is likely to have an anti-tumor response to a first therapeutic agent comprising (a) a one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, and (II) treating the patient with a therapeutic regimen that comprises the first therapeutic agent if the biomarker is present or treating the patient with a therapeutic regimen that does not include the first therapeutic agent if the biomarker is absent; wherein the biomarker comprises a subset of TME gene signature comprising a Tertiary Lymphoid Structures (TLS) signature; wherein the TLS signature comprises a gene CCL18, CCL19, CCL21, CXCL13, LAMP3, LTB, MS4A1, or combinations thereof.

In some embodiments, provided herein is a method for testing a patient having a tumor for the presence or absence of a baseline biomarker that predicts that the patient is likely to have an anti-tumor response to a treatment with a therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, the method comprising: (I) obtaining a baseline sample that has been isolated from the tumor of the patient; (II) measuring the baseline expression level of each gene in a tumor microenvironment (TME) gene or a subset of said genes; (III) normalizing the measured baseline expression levels; (IV) calculating a baseline signature score for the TME gene signature from the normalized expression levels; (V) comparing the baseline signature score to a reference score for the TME gene signature; and (VI) classifying the patient as biomarker positive or biomarker negative for an outcome related to a durable clinical benefit (DCB) from the therapeutic agent.

In some embodiments, the representative sample from the tumor of the patient is isolated on day 0, or at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, or at least 1 month, 2 months, 3 months, 4 months, 5 months, 6 months, 1 year or at least 2 years after administering the therapeutic, wherein the therapeutic is the first therapeutic.

In some embodiments, the method described herein can be used to determine qualitative assessment of the neoantigen specific T cell population expanded ex vivo for suitability as a therapeutic cell population comprising neoantigen specific cytotoxic T cells. Therefore, provided herein is a method for determining induction of tumor neoantigen specific T cells in a tumor, the method comprising: detecting one or more tumor microenvironment (TME) signatures of durable clinical benefit (DCB) comprising: a B-cell signature, a Tertiary Lymphoid Structures (TLS) signature, an effector/memory-like CD8+ T cell signature, a HLA-E/CD94 interaction signature, a NK cell signature, and an MHC class II signature, wherein at least one of the signatures is altered compared to a corresponding representative sample before administering the composition.

In one embodiment, provided herein is a method of testing a patient having a cancer or a tumor for the presence or absence of an on-treatment biomarker that predicts that the patient is likely to have an anti-tumor response to administering a first therapeutic agent comprising (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein, the method comprising:

obtaining a representative baseline sample from a tumor collected from the patient;

measuring in the baseline sample a baseline expression level of each gene in a tumor microenvironment (TME) signature;

normalizing the measured baseline expression levels;

calculating a baseline TME gene signature score for the TME gene signature from the normalized baseline expression levels;

obtaining a representative sample from the tumor that has been collected from the patient at a time post-treatment;

measuring the post-treatment expression level of each gene in the TME gene signature in representative sample from the tumor that has been collected from the patient at a time period post-treatment;

normalizing each of the measured post-treatment expression levels;

calculating a post-treatment TME gene signature score for each gene in the TME gene signature from the normalized expression levels;

calculating a post-treatment TME gene signature score for each gene in the TME gene signature from the measured expression levels;

comparing the post-treatment TME gene signature score to the baseline TME gene signature score, and

classifying the patient as biomarker positive or biomarker negative for an outcome related to durable clinical benefit (DCB) from the first therapeutic agent;

wherein obtaining, measuring, normalizing and calculating the baseline TME gene signature score can be performed before or concurrently with obtaining, measuring, normalizing and calculating the post-treatment TME gene signature score; and

wherein a biomarker positive patient is determined to be likely experience a DCB with the first therapeutic agent.

In some embodiments a durable clinical benefit comprises that the patient is progression free for 2 months, or 3 months, 4 months, 5 months, 6 months, 7 months, 8 months, 9 months, 10 months, 11 months, or 12 months.

In some embodiments a durable clinical benefit comprises that the patient is progression free for 1 year, or 2 years, 3 years, 5 years, 6 years, 7 years, 8 years, 9 years, 10 years, 11 years, or 12 years.

In some embodiments the therapeutic is a tumor neoantigen vaccine.

### Embodiments

1. In one embodiment, provided herein is a method of treating a patient having a tumor comprising:
determining if a sample collected from the patient is positive or negative for a biomarker which predicts that the patient is likely to have an anti-tumor response to a first therapeutic agent comprising (i) a one or more peptides comprising a neoepitope of a protein,
i.(ii) a polynucleotide encoding the one or more peptides,
ii.(iii) one or more APCs comprising the one or more peptides or the polynucleotide encoding the one or more peptides, or
iii.(iv) a T cell receptor (TCR) specific for a neoepitope of the one or more peptides in complex with an HLA protein, and
   treating the patient with a therapeutic regimen that comprises the first therapeutic agent if the biomarker is present; or treating the patient with a therapeutic regimen that does not include the first therapeutic agent if the biomarker is absent, wherein the biomarker comprises a tumor microenvironment (TME) signature.
   2. The method of embodiment 1, wherein the TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, an MHC class II signature or a functional Ig CDR3 signature.
   3. The method of embodiment 1 or 2, wherein the B-cell signature comprises expression of a gene comprising CD20, CD21, CD3, CD22, CD24, CD27, CD38, CD40, CD72, CD79a, IGKC, IGHD, MZB1, MS4A1, CD138, BLK, CD19, FAM30A, FCRL2, MS4A1, PNOC, SPIB, TCL1A, TNFRSF17 or combinations thereof.
   4. The method of embodiment 1 or 2, wherein the TLS signature indicates formation of tertiary lymphoid structures.
   5. The method of embodiment 1 or 2, wherein the tertiary lymphoid structure represents aggregates of lymphoid cells.
   6. The method of embodiment 1 or 2, wherein the TLS signature comprises expression of a gene comprising CCL18, CCL19, CCL21, CXCL13, LAMP3, LTB, MS4A1, or combinations thereof.
   7. The method of embodiment 1 or 2, wherein the TIS signature comprises an inflammatory gene, a cytokine, a chemokine, a growth factor, a cell surface interaction protein, a granulation factor, or a combination thereof.
   8. The method of embodiment 1 or 2, wherein the TIS signature comprises CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT or a combination thereof.
   9. The method of embodiment 1 or 2, wherein the effector/memory-like CD8+ T cell signature comprises expression of a gene comprising CCR7, CD27, CD45RO, CCR7, FLT3LG, GRAP2, IL16, IL7R, LTB, S1PR1, SELL, TCF7, CD62L, or any combination thereof.
   10. The method of embodiment 1 or 2, wherein the HLA-E/CD94 signature comprises expression of a gene CD94 (KLRD1), CD94 ligand, HLA-E, KLRC1 (NKG2A), KLRB1 (NKG2C) or any combination thereof.
   11. The method of embodiment 1 or 2, wherein the HLA-E/CD94 signature further comprises an HLA-E:CD94 interaction level.
   12. The method of embodiment 1 or 2, wherein the NK cell signature comprises expression of a gene CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2 or a combination thereof.
   13. The method of embodiment 1 or 2, wherein the MHC class II signature comprises expression of a gene that is an HLA comprising HLA-DMA, HLA-DOA, HLA-DPA1, HLA-DPB1, HLA-DQB1, HLA-DRA, HLA-DRB1, HLA-DRB5 or a combination thereof.
   14. The method of embodiment 1 or 2, wherein the biomarker comprises a subset of TME gene signature comprising a Tertiary Lymphoid Structures (TLS) signature; wherein the TLS signature comprises a gene CCL18, CCL19, CCL21, CXCL13, LAMP3, LTB, MS4A1, or combinations thereof.
   15. The method of embodiment 1 or 2, wherein the functional Ig CDR3 signature comprises an abundance of functional Ig CDR3 s.
   16. The method of embodiment 15, wherein the abundance of functional Ig CDR3s is determined by RNA-seq.
   17. The method of embodiment 15 or 16, wherein the abundance of functional Ig CDR3s is an abundance of functional Ig CDR3s from cells of a TME sample from a subject.
   18. The method of any one of embodiments 15-17, wherein the abundance of functional Ig CDR3s is 2\hat{ }7 or more functional Ig CDR3s.
   19. The method of any one of the embodiments 1-18, wherein the method further comprises: administering to the biomarker positive patient the first therapeutic agent, an altered dose or time interval of the first therapeutic agent, or a second therapeutic agent.
   20. The method of any one of the embodiments 1-18, wherein the method further comprises: not administering to the biomarker negative patient the first therapeutic agent or a second therapeutic agent.
   21. The method of any one of the embodiments 1-18, wherein the method further comprises administering to the biomarker positive patient, an increased dose of the first therapeutic agent.
   22. The method of any one of the embodiments 1-18, wherein the method further comprises modifying a time interval of administration of the first therapeutic agent to the biomarker positive or negative patient.
   23. In one embodiment, provided herein is a method for testing a patient having a tumor for the presence or absence of a baseline biomarker that predicts that the patient is likely to have an anti-tumor response to a treatment with a therapeutic agent comprising one or more peptides comprising a neoepitope of a protein,
   (ii) a polynucleotide encoding the one or more peptides,
   (iii) one or more APCs comprising the one or more peptides or the polynucleotide encoding the one or more peptides, or
   (iv) a T cell receptor (TCR) specific for a neoepitope of the one or more peptides in complex with an HLA protein, the method comprising:
   (a) obtaining a baseline sample that has been isolated from the tumor of the patient; measuring the baseline expression level of each gene in a tumor microenvironment (TME) gene or a subset of said genes;
   (b) normalizing the measured baseline expression levels; calculating a baseline signature score for the TME gene signature from the normalized expression levels;
   (c) comparing the baseline signature score to a reference score for the TME gene signature; and,
   (d) classifying the patient as biomarker positive or biomarker negative for an outcome related to a durable clinical benefit (DCB) from the therapeutic agent.
   24. The method of embodiment 23, wherein the TME signature comprises a signature of one or more of embodiments 2-18, or a subset thereof.
   25. In one embodiment, provided herein is a pharmaceutical composition for use in treating cancer in a patient who tests positive for a biomarker, wherein the composition the therapeutic agent comprises (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein; and at least one pharmaceutically acceptable excipient; and wherein the biomarker is an on-treatment biomarker which comprises a gene signature selected from the group consisting of TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MEW class II signature.
   26. The pharmaceutical composition of embodiment 25, wherein the TME signature comprises a signature of any one or more of embodiments 2-18, or a subset thereof.
   27. In one embodiment, provided herein is a method of treating cancer in a subject in need thereof, comprising: administering a therapeutically effective amount of a cancer therapeutic agent, wherein the subject has an increased likelihood of responding to the cancer therapeutic agent, wherein the subject's increased likelihood of responding to the cancer therapeutic agent is associated with the presence of one or more peripheral blood mononuclear cell signatures prior to treatment with the cancer therapeutic agent; and wherein at least one of the one or more peripheral blood mononuclear cell signatures comprises a threshold value for a ratio of cell counts of a first mononuclear cell type to a second mononuclear cell type in the peripheral blood of the subject.
   28. The method of embodiment 27, wherein the cancer is melanoma.
   29. The method of embodiment 27, wherein the cancer is non-small cell lung cancer 30. The method of embodiment 27, wherein the cancer is bladder cancer.
   31. The method of embodiment 27, wherein the cancer therapeutic comprises a neoantigen peptide vaccine.
   32. The method of embodiment 27, wherein the cancer therapeutic comprises an anti-PD1 antibody.
   33. The method of embodiment 27, wherein the cancer therapeutic comprises a combination of the neoantigen vaccine and the anti-PD1 antibody, wherein the neoantigen vaccine is administered or co-administered after a period of administering anti-PD1 antibody alone.
   34. The method of embodiment 32 or 33, wherein the anti-PD1 antibody is nivolumab.
   35. The method of embodiment 27, wherein the threshold value is a maximum threshold value.
   36. The method of embodiment 27, wherein the threshold value is a minimum threshold value.
   37. The method of embodiment 27, wherein at least one of the one or more peripheral blood mononuclear cell signatures comprises a maximum threshold value for a ratio of naïve CD8+ T cells to total CD8+ T cells in a peripheral blood sample from the subject.
   38. The method of embodiment 37, wherein the maximum threshold value for the ratio of naïve CD8+ T cells to total CD8+ T cells in the peripheral blood sample from the subject is about 20:100.
   39. The method of embodiment 37 or 38, wherein the peripheral blood sample from the subject has a ratio of naïve CD8+ T cells to total CD8+ T cells that is 20:100 or less or less than 20:100.
   40. The method of embodiment 27, wherein at least one of the one or more peripheral blood mononuclear cell signatures comprises a minimum threshold value for a ratio of effector memory CD8+ T cells to total CD8+ T cells in a peripheral blood sample from the subject.
   41. The method of embodiment 40, wherein the minimum threshold value for the ratio of effector memory CD8+ T cells to total CD8+ T cells in the peripheral blood sample from the subject is about 40:100.
   42. The method of embodiment 40 or 41, wherein the peripheral blood sample from the subject has a ratio of effector memory CD8+ T cells to total CD8+ T cells that is 40:100 or more or more than 40:100.
   43. The method of embodiment 27, wherein at least one of the one or more peripheral blood mononuclear cell signatures comprises a minimum threshold value for a ratio of class-switched memory B cells to total CD19+ B cells in a peripheral blood sample from the subject.
   44. The method of embodiment 43, wherein the minimum threshold value for the ratio of class-switched memory B cells to total CD19+ B cells in the peripheral blood sample from the subject is about 10:100.
   45. The method of embodiment 43 or 44, wherein the peripheral blood sample from the subject has a ratio of class-switched memory B cells to total CD19+ B cells that is 10:100 or more or more than 10:100.
   46. The method of embodiment 27, wherein at least one of the one or more peripheral blood mononuclear cell signatures comprises a maximum threshold value for a ratio of naïve B cells to total CD19+ B cells in a peripheral blood sample from the subject.
   47. The method of embodiment 46, wherein the maximum threshold value for the ratio of naïve B cells to total CD19+ B cells in the peripheral blood sample from the subject is about 70:100.
   48. The method of embodiment 46 or 47, wherein the peripheral blood sample from the subject has a ratio of naïve B cells to total CD19+ B cells that is 70:100 or less or less than 70:100.
   49. The method of any one of the embodiments 37-48, wherein the cancer is a melanoma.
   50. The method of embodiment 27, wherein at least one of the one or more peripheral blood mononuclear cell signatures comprises a maximum threshold value for a ratio of plasmacytoid dendritic cells to total Lin−/CD11c− cells in a peripheral blood sample from the subject.
   51. The method of embodiment 50, wherein the maximum threshold value for the ratio of plasmacytoid dendritic cells to total Lin−/CD11c− cells in the peripheral blood sample from the subject is about 3:100.
   52. The method of embodiment 50 or 51, wherein the peripheral blood sample from the subject has a ratio of plasmacytoid dendritic cells to total Lin−/CD11c− cells that is 3:100 or less or less than 3:100.
   53. The method of embodiment 27, wherein at least one of the one or more peripheral blood mononuclear cell signatures comprises a maximum threshold value for a ratio of CTLA4+ CD4 T cells to total CD4+ T cells in a peripheral blood sample from the subject
   54. The method of embodiment 50, wherein the maximum threshold value for the ratio of CTLA4+ CD4 T cells to total CD4+ T cells in the peripheral blood sample from the subject is about 9:100.
   55. The method of embodiment 50 and 51, wherein the peripheral blood sample from the subject has a ratio of CTLA4+ CD4 T cells to total CD4+ T cells that is 9:100 or less or less than 9:100.
   56. The method of any one of the embodiments 50-55, wherein the cancer is a non-small cell lung cancer.
   57. The method of embodiment 27, wherein at least one of the one or more peripheral blood mononuclear cell signatures comprises a minimum threshold value for a ratio of memory CD8+ T cells to total CD8+ T cells in a peripheral blood sample from the subject.
   58. The method of embodiment 57, wherein the minimum threshold value for the ratio of memory CD8+ T cells to total CD8+ T cells in the peripheral blood sample from the subject is about 40:100 or about 55:100.
   59. The method of embodiment 57 and 58, wherein the peripheral blood sample from the subject has a ratio of memory CD8+ T cells to total CD8+ T cells that is 40:100 or more or more than 40:100
   60. The method of embodiment 57 and 58, wherein the peripheral blood sample from the subject has a ratio of memory CD8+ T cells to total CD8+ T cells that is 55:100 or more or more than 55:100.
   61. The method of any one of the embodiments 57-60, wherein the cancer is a bladder cancer.
   62. In one embodiment, provided herein is a method of treating cancer in a subject in need thereof, comprising: administering to the subject a therapeutically effective amount of a cancer therapeutic agent, wherein the subject has an increased likelihood of responding to the cancer therapeutic agent, and wherein the subject's increased likelihood of responding to the cancer therapeutic agent is associated with a clonal composition characteristic of TCR repertoires analyzed from peripheral blood sample of the subject at least at a timepoint prior to administering the cancer therapeutic agent.
   63. The method of embodiment 62, wherein the clonal composition characteristic of TCR repertoires in a prospective patient is defined by a relatively low TCR diversity versus the TCR diversity in healthy donors.
   64. The method of embodiment 62 or 63, wherein the clonal composition characteristic is analyzed by a method comprising sequencing the TCRs or fragments thereof.
   65. The method of embodiment 62, wherein the clonal composition characteristic of TCR repertoires is defined by the clonal frequency distribution of the TCRs.
   66. The method of any one of the embodiments 62-65, wherein the clonal composition characteristic of the TCR repertoires is further analyzed by calculating the frequency distribution pattern of TCR clones.
   67. The method of embodiment 66, wherein the frequency distribution pattern of TCR clones is analyzed using one or more of: Gini Coefficient, Shannon entropy, DE50, Sum of Squares, and Lorenz curve.
   68. The method of embodiment 62, wherein the subject's increased likelihood of responding to the cancer therapeutic agent is associated with increased clonality of the TCRs.
   69. The method of embodiment 62, wherein the subject's increased likelihood of responding to the cancer therapeutic agent is associated with increased frequency of medium and/or large and/or hyperexpanded sized TCR clones.
   70. The method of embodiment 62, wherein the subject's increased likelihood of responding to the cancer therapeutic agent is associated with a clonal composition characteristic of TCR repertoires according to any one of embodiments 63-69, wherein the clonal composition characteristic is analyzed from peripheral blood sample of the subject prior to administering a therapeutically effective amount of a cancer therapeutic agent.
   71. The method of embodiment 62, wherein a clonal composition characteristic of TCR repertoires comprises a measure of the clonal stability of the TCRs.
   72. The method of embodiment 70 or 71, wherein the clonal stability of the TCRs is analyzed as TCR turnover between a first and a second timepoints, wherein the first timepoint is prior to administering the cancer therapeutic agent and the second timepoint is a timepoint during the duration of the treatment.
   73. The method of embodiment 71, wherein the second timepoint is prior to administering the vaccine.
   74. The method of embodiment 70, wherein the clonal stability of TCRs is analyzed using a Jensen-Shannon Divergence.
   75. The method of embodiment 70, wherein the subject's increased likelihood of responding to the cancer therapeutic agent is associated with higher TCR stability.
   76. The method of embodiment 70, wherein the subject's increased likelihood of responding to the cancer therapeutic agent is associated with reduced turnover of T cell clones between the first timepoint and the second timepoint.
   77. In one embodiment, provided herein is a method of treating cancer in a subject in need thereof, comprising: administering a therapeutically effective amount of a cancer therapeutic agent to the subject, wherein the subject has an increased likelihood of responding to the cancer therapeutic agent, wherein the subject's increased likelihood of responding to the cancer therapeutic agent is associated with the presence of one or more genetic variations in the subject, wherein the subject has been tested for a presence of the one or more genetic variations with an assay and has been identified as having the one or more genetic variations, wherein the one or more genetic variations comprise an ApoE allele genetic variation comprising (i) an ApoE2 allele genetic variation comprising a sequence encoding a R158C ApoE protein or (ii) an ApoE4 allele genetic variation comprising a sequence encoding a C112R ApoE protein.
   78. The method of embodiment 77, wherein the cancer therapeutic agent comprises a neoantigen peptide vaccine.
   79. The method of embodiment 77, wherein the cancer therapeutic agent further comprises an anti-PD1 antibody.
   80. The method of embodiment 77, wherein the cancer therapeutic agent does not comprise an anti-PD1 antibody monotherapy.
   81. The method of embodiment 77, wherein the cancer is melanoma.
   82. The method of embodiment 77, wherein the subject is homozygous for the ApoE2 allele genetic variation.
   83. The method of embodiment 77, wherein the subject is heterozygous for the ApoE2 allele genetic variation.
   84. The method of embodiment 77, wherein the subject is homozygous for the ApoE4 allele genetic variation.
   85. The method of embodiment 77, wherein the subject is heterozygous for the ApoE4 allele genetic variation.
   86. The method of embodiment 77, wherein the subject comprises an ApoE allele comprising a sequence encoding a ApoE protein that is not a R158C ApoE protein or a C112R ApoE protein.
   87. The method of embodiment 77, wherein the subject has rs7412-T and rs449358-T.
   88. The method of embodiment 77, wherein the subject has rs7412-C and rs449358-C.
   89. The method of embodiment 77, wherein a reference subject that is homozygous for the ApoE3 allele has a decreased likelihood of responding to the cancer therapeutic agent.
   90. The method of embodiment 77, wherein the assay is a genetic assay.
   91. The method of embodiment 77, wherein the cancer therapeutic agent comprises one or more peptides comprising a cancer epitope.
   92. The method of embodiment 77, wherein the cancer therapeutic agent comprises (i) a polynucleotide encoding the one or more peptides of embodiment 91,
   (i) or, (ii) one or more APCs comprising the one or more peptides or the polynucleotide encoding the one or more peptides,
   (ii) or (iii) a T cell receptor (TCR) specific for a cancer epitope of the one or more peptides in complex with an HLA protein.
   93. The method of any one of the embodiments 77-92, wherein the cancer therapeutic agent further comprises an immunomodulatory agent.
   94. The method of embodiment 93, wherein the immunotherapeutic agent is an anti-PD1 antibody.
   95. The method of embodiment 77, wherein the cancer therapeutic agent is not nivolumab alone or pembrolizumab alone.
   96. The method of embodiment 77, wherein the one or more genetic variations comprises chr19:44908684 T>C; wherein chromosome positions of the one or more genetic variations are defined with respect to UCSC hg38.
   97. The method of embodiment 77, wherein the one or more genetic variations comprises chr19:44908822 C>T; wherein chromosome positions of the one or more genetic variations are defined with respect to UCSC hg38.
   98. The method of embodiment 77, wherein the method further comprises testing the subject for the presence of the one or more genetic variations with the assay prior to the administering.
   99. The method of embodiment 77, wherein the ApoE2 allele genetic variation is a germline variation.
   100. The method of embodiment 77, wherein the ApoE4 allele genetic variation is a germline variation.
   101. The method of embodiment 77, wherein the method comprises administering to the subject a cancer therapeutic agent comprising one or more peptides comprising a cancer epitope; wherein the subject is determined as having the germline ApoE4 allelic variant.
   102. The method of embodiment 101, wherein the therapeutic agent further comprises one or more of: an adjuvant therapy, a cytokine therapy, or an immunomodulator therapy.
   103. The method of embodiment 101 or 102, wherein the immunomodulator therapy is a PD1 inhibitor, such as an anti-PD1 antibody.
   104. The method of any one of the embodiments 101-103, wherein the therapeutic agent does not comprise a PD1 inhibitor monotherapy.
   105. The method of embodiment 77, wherein the method further comprises administering an agent that promotes ApoE activity or comprises ApoE activity.
   106. The method of embodiment 77, wherein the method further comprises administering an agent that inhibits ApoE activity.
   107. The method of any one of the preceding embodiments, where the cancer is a pancreatic cell cancer.
   108. The method of any one of the preceding embodiments, wherein the therapeutic agent comprises a vaccine.
   109. The method of any one of the preceding embodiments, wherein the therapeutic agent comprises a peptide vaccine, comprising at least one, two, three or four antigenic peptides.
   110. The method of any one of the preceding embodiments, wherein the therapeutic agent comprises a peptide vaccine, comprising at least one, two, three or four neoantigenic peptides.
   111. The method of any one of the preceding embodiments, wherein the therapeutic agent comprises a nucleic acid encoding a peptide, wherein the peptide is a neoantigen peptide.
   112. The method of any one of the preceding embodiments, wherein the therapeutic agent comprises a combination therapy comprising one or more checkpoint inhibitor antibodies, and a vaccine comprising a neoantigen peptide, or a nucleic acid encoding the neoantigenic peptide.
   113. The method of embodiment 70, wherein the clonal composition characteristic is analyzed from peripheral blood sample of the subject prior to administering a vaccine, wherein the vaccine comprises at least one peptide or a polynucleotide encoding a peptide, wherein the cancer therapeutic agent comprises a combination of a neoantigen vaccine and an anti-PD1 antibody, wherein the neoantigen vaccine is administered or co-administered after a period of administering anti-PD1 antibody alone.

### EXAMPLES

### Example 1. Methods of TME Signature Analysis

In this and the following examples, tumor samples were collected from melanoma patients who were treated with a neoantigen vaccine NEO-PV-01 in combination with nivolumab (anti PD-1 therapy, immune checkpoint inhibitor) and TME were identified from subjects who had durable clinical benefit and those who did not have durable clinical benefit. NEO-PV-01 is composed of a mixture of up to 20 unique neoantigen peptides of 14-35 amino acids in length. Peptides are pooled together in four groups of up to five peptides each, and mixed with an adjuvant at the time of administration. NT-001 is a phase 1B trial of NEO-PV-01 in combination with nivolumab, in patients with unresectable or metastatic melanoma, non-small cell lung cancer (NSCLC), and transitional cell carcinoma (TCC) of the bladder (NCT02897765). Both peripheral blood (PBMCs) and tumor samples are collected from the patient at the following timepoints (FIG. 1) Tumor biopsies from all three tumor types were collected i) prior to treatment (pre-treatment, i.e., Week 0 pre-Nivolumab), ii) after 12 weeks of nivolumab monotherapy (pre-vaccine); and iii) after completion of NEO-PV-01+nivolumab vaccination (post-vaccine).

Three leukapheresis samples were taken at week 0 (pre-treatment, preT), week 10 (pre-vaccination, preV), and week 20 (post-vaccination, postV) (FIG. 1A). First, RNA was extracted from peripheral blood CD3+ T cells and subjected to T cell receptor (3-chain (TCRβ) sequencing. We analyzed a total of 57 samples from 21 of the 34 melanoma patients in the trial for whom we had samples from at least one time-point. 14 patients had a durable clinical benefit (DCB, defined as PFS ≥9 months) and 7 did not (tumor staging, and additional characteristics are found in Table S1).

Tumor biopsies were analyzed for multiple immune and tumor markers by immunohistochemistry and targeted gene expression. Targeted gene expression analysis on RNA extracted from FFPE blocks was performed using the NanoString™ nCounter platform. A custom set of 800 genes included markers for immune cell populations, cytolytic markers, immune activation and suppression, and the tumor microenvironment. Gene signatures of key immune features were calculated after normalization with housekeeping genes and used for subsequent analysis. If the maximum tumor content from multiple blocks of a single biopsy is lower than 20% (determined by IHC), the biopsy is noted as low tumor content, or <20% tumor.

### Patient Characteristics

Melanoma Patients used for tumor biopsy analysis were part of the NT001 safety cohort, in which every patient had received at least one dose of NEO-PV-01 at time of data reporting. Patients who met the 36 week progression free survival (PFS) milestone are classified in the Durable Clinical Benefit (DCB) group. Patients who did not meet the 36 week PFS milestone are classified in the no DCB Group. Table 2A shows the grouping of the patients based on outcome. Table 2B shows demographic features of the patient cohort for NT001 study. Table 2C provides data on patient's age, sex and sample sizes for TCR analysis, and also the DCB status.

TABLE 2A
Study design and DCB in melanoma cohort

| | | | Tumor Biopsy | | |
|---|---|---|---|---|---|
| | | Safety Cohort | | Nivolumab | NEO~PV~01 + |
| Indication | 36~week~PFS | Subject | Pre~Treatment | Monotherapy | Nivolumab |
| MELANOMA | DCB | 11 | 11 | 10 | 9 |
| | no DCB | 8 | 7 | 6 | 4 |

TABLE 2B
Patient characteristics at enrollment

| | Patients Initiated Vaccine (n = 23) |
|---|---|
| Tumor PD-L1 Expression | |
| >1% | 65% |
| >50% | 12% |
| Tumor Mutation Burden, median (range) | 364 (57-8433) |
| Prior Systemic Therapy | 35% |
| ECOG performance status | |
| 0 | 83% |
| 1 | 18% |
| Metastatic Lesions (%) | |
| M0 | 0% |
| M1a | 26% |
| M1b & M1c | 74% |
| Common melanoma driver mutations | |
| BRAF* | 17 |
| NRAS | 17 |
| NF1 | 35 |

TABLE 2C
Table providing the age, sex, DCB status and sample availability
for TCR sequencing at each point

| | | | | Pre- | Pre- | Post- |
|---|---|---|---|---|---|---|
| | | | | Treatment | Vaccine | Vaccine |
| Patient | Age | Sex | DCB | Sample | Sample | Sample |
| M1 | 55 | F | Yes | 1 | 1 | 1 |
| M10 | 63 | M | Yes | 1 | 1 | 1 |
| M12 | 63 | M | Yes | 1 | 1 | 1 |
| M13 | 60 | M | Yes | 1 | 1 | 1 |
| M14 | 77 | M | Yes | 1 | 1 | 1 |
| M15 | 80 | F | No | 1 | 1 | 1 |
| M16 | 25 | M | No | 0 | 0 | 1 |
| M17 | 37 | M | No | 1 | 1 | 1 |
| M18 | 71 | M | Yes | 1 | 1 | 1 |
| M2 | 65 | M | Yes | 1 | 1 | 1 |
| M20 | 59 | M | No | 1 | 1 | 1 |
| M22 | 47 | F | Yes | 1 | 0 | 1 |
| M23 | 67 | M | Yes | 1 | 1 | 1 |
| M3 | 62 | M | No | 1 | 1 | 1 |
| M4 | 52 | F | No | 1 | 1 | 1 |
| M5 | 57 | M | Yes | 1 | 1 | 1 |
| M6 | 54 | F | Yes | 1 | 1 | 1 |
| M7 | 84 | M | Yes | 1 | 1 | 1 |
| M8 | 59 | M | Yes | 1 | 1 | 1 |
| M9 | 50 | F | Yes | 1 | 1 | 0 |
| NV10 | 59 | M | No | 1 | 0 | 0 |

### Peripheral Sample Flow Cytometry Staining Protocol:

Patient PBMCs were thawed into FBS, followed by a wash with Lonza X-VIVO 15 media to remove cells from DMSO. Cells were then treated with benzonase for 30 minutes at a 1:1000 dilution in media at 37° C. Cells were washed with media and counted using the Guava easyCyte Flow cytometer. 2*10\hat{ }6 cells per sample were plated for flow staining and washed once with FACS buffer (PBS+0.5% BSA). Cells were then incubated with surface stain antibody cocktails listed above for 30 minutes on ice, followed by a wash with FACS buffer. Next, cells were fixed and permeabilized for intracellular staining using one of two methods (depending on the panel) for 20 minutes on ice. All cells stained using the B cell panel were fixed and permeabilized using the BD cytofix/cytoperm kit according the manufacturer's instructions. All cells stained with the T cell panel were fixed and permeabilized using the Invitrogen FOXP3 staining buffer set Fixation/Permeabilization concentrate and diluent according to the manufacturer's instructions. Cells were washed with the corresponding permeabilization wash buffer according to the manufacturer's instructions. Cells were then incubated with intracellular antibodies in the corresponding permeabilization wash buffer for 30 minutes on ice, washed with the appropriate permeabilization wash buffer, followed by a final wash with FACS buffer. Cells were stored in FACS buffer at 4° C. until analysis on a BD LSR Fortessa flow cytometer.

T Cell Panel:

CD3 BV421 (Sk7), CD19 APCCy7 (791), CD4 BUV496 (SK3), CD8 BUV805 (SK1), CD45RO BV605 (UCHL1), CD45RA AF700 (HI100), CD62L FITC (DREG-56), CD27 BV711 (M-T271), ICOS BUV396 (DX29), CD137 BV650 (4B4-1), CD69 BV786 (FN50), PD-1 BV510 (EH12.1), CD26 PECF594 (M-A261), CD25 PerCPCy5.5 (M-A251), CTLA4 PECy5 (BNI3) and TCF7 PE (S33-966) from BD Biosciences; Gamma-9 APC (B3) from BioLegend; FOXP3 PECy7 (PCH101) and Live/Dead APCCy7 from Invitrogen.

B Cell Panel:

CD19 BUV496 (SJ25C1), CD20 BUV805 (2H7), IgK light chain AF700 (G20-193), CD138 PE (MI15), CD27 BV786 (L128), IgD BV605 (1A6-2), CD1c BV421 (F10/21A3), IgM BUV396 (G20-127), and CD24 BV650 (ML5) from BD Biosciences, CD3 FITC (HIT3a), CD56 FITC (5.1H11), CD14 FITC (M5E2), CD38 BV711 (HIT2), CD269 PECF594 (19F2), IgL light chain PerCPCy5.5 (MHL-38), CD22 BV510 (HIB22), CD267 APC (1A1), HLA-DR PeCy5 (L243), and CD79a PECy7 (HM47) from Biolegend; and Live/Dead APCCy7 from Invitrogen.

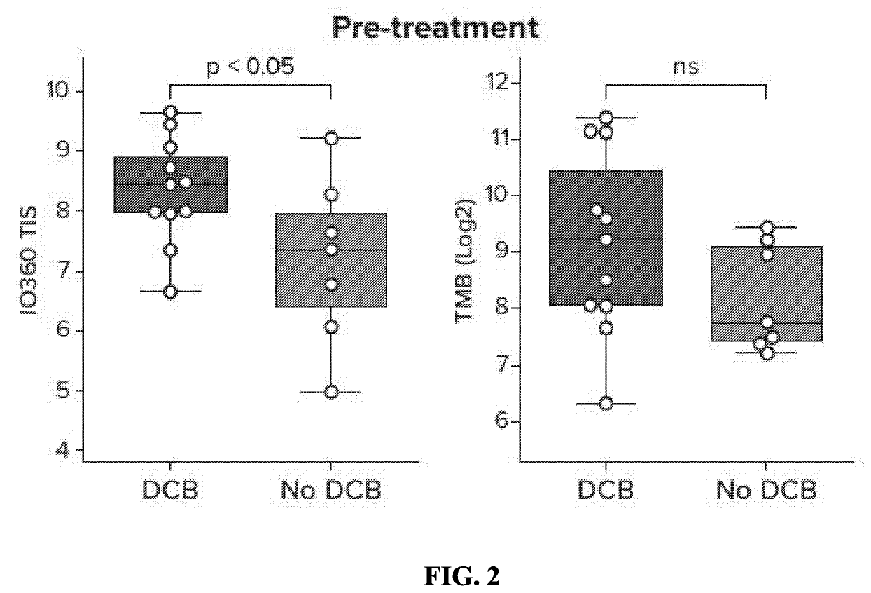

### Example 2. TME-TIS Score is Associated with DCB in Melanoma Patients (See FIG. 2, Left)

In this example an 18-gene TIS signature that measures a pre-existing but suppressed adaptive immune response within tumors was investigated comparing between DCB and no-DCB in the melanoma patients prior to treatment (pre-treatment), after 12 weeks of nivolumab monotherapy (pre-vaccine), and after completion of NEO-PV-01+nivolumab vaccination (post-vaccine). Results shown in FIG. 2 (left) indicate that the TIS signature is enriched in melanoma patients d with DCB. It was also noted that tumor mutational burden (TMB) is not associated with DCB in melanoma patients (FIG. 2, right panel).

### Example 3. Memory and Effector T Cell-Like TCF7+ CD8+ T Cells Associated with TME Signature was Increased in Melanoma Patients with DCB

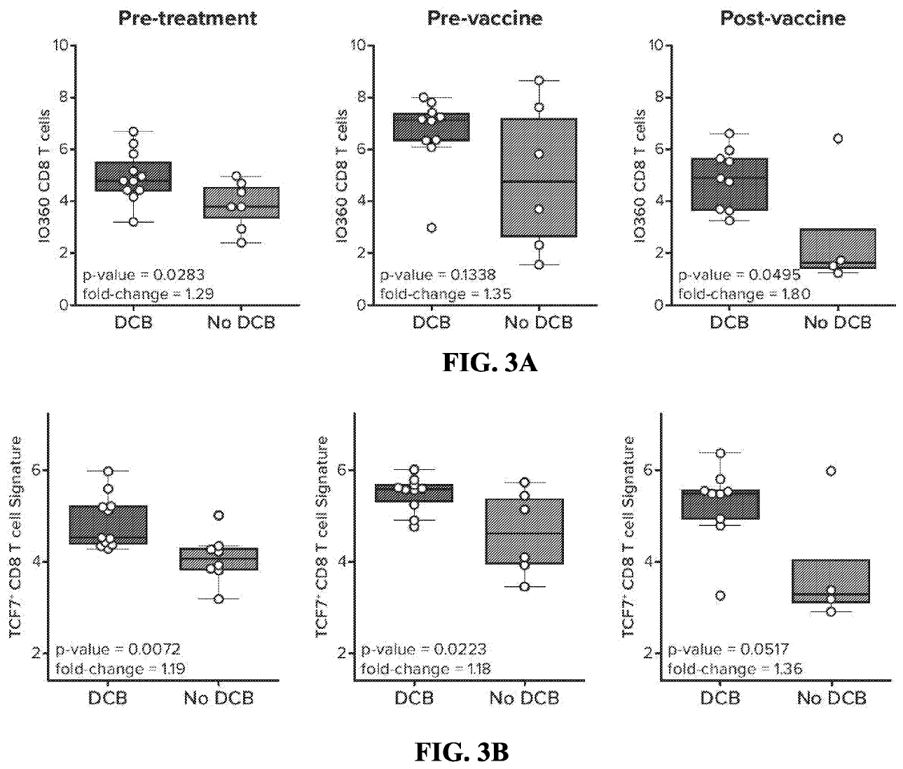

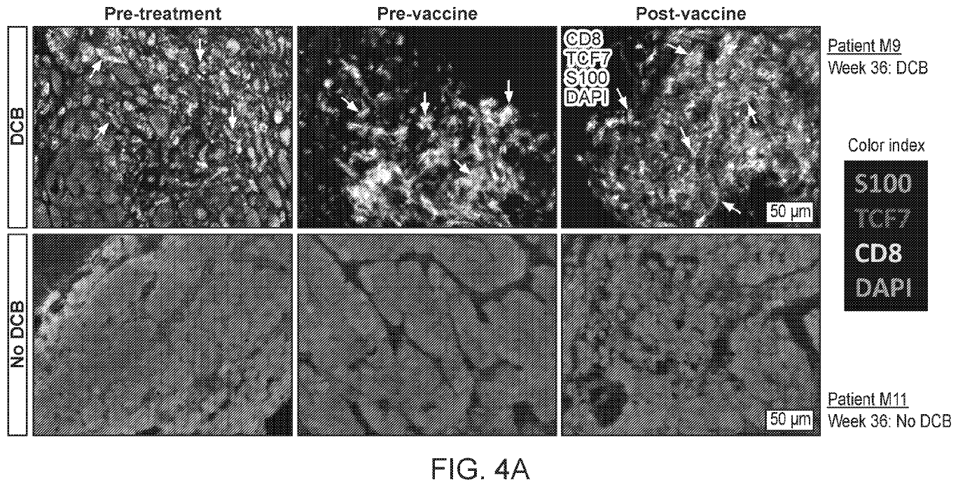

In this exemplary study, specific T cell signatures were analyzed in tumor biopsy samples prior to treatment (pre-treatment), after 12 weeks of nivolumab monotherapy (pre-vaccine), and after completion of NEO-PV-01+nivolumab vaccination (post-vaccine), in which every patient had received at least one dose of NEO-PV-01 at time of data reporting (FIG. 3A-3B). Patients with DCB have increased expression of CD8+ T cell genes at the pre-treatment time point (FIG. 3A).

FIG. 3B shows that memory and/or effector-like TCF7+ CD8+ T cell signature is increased in melanoma patients with DCB. The memory and/or effector-like TCF7+ CD8 T cell associated signature was derived from CD8+ T cell sub-clusters that express genes consistent with a memory- and/or effector-like phenotype and express the stem-like transcription factor TCF7; higher expression of this gene signature is associated with DCB and predicts outcome of metastatic melanoma patients. Melanoma patients with DCB demonstrated increased numbers of TC7+ CD8+ T cells in the tumor microenvironment compared to patients that had no DCB.

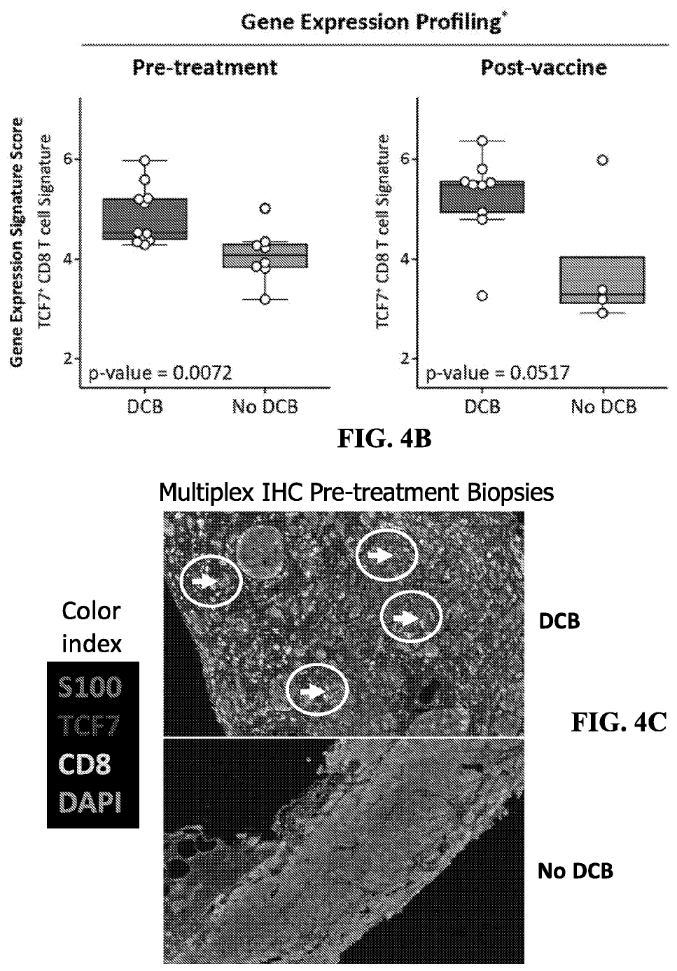

Upon performing immunohistochemistry, the data corresponded with the findings in FIG. 3B (FIG. 4A). Markers for CD8+ T cells, TCF7, and tumor cells (S100) were simultaneously used to examine expression of TCF7 in CD8+ T cells in patients with DCB and no DCB prior to treatment (pre-treatment), after 12 weeks of nivolumab monotherapy (pre-vaccine), and after completion of NEO-PV-01+nivolumab vaccination (post-vaccine). A representative patient from each cohort is shown. CD8+ TCF7+ T cells are indicated by white arrows. What was further observed is that the difference with respect to these markers were clearly distinct between DCB and No DCB patients at the pre-treatment timepoint (FIGS. 4B and 4C), which emphasizes its predictive value of the signatures prior to commencement of NEO-PV-01+nivolumab.

### Example 4. Higher TME B-Cells Signature is Associated with DCB in Melanoma Patients

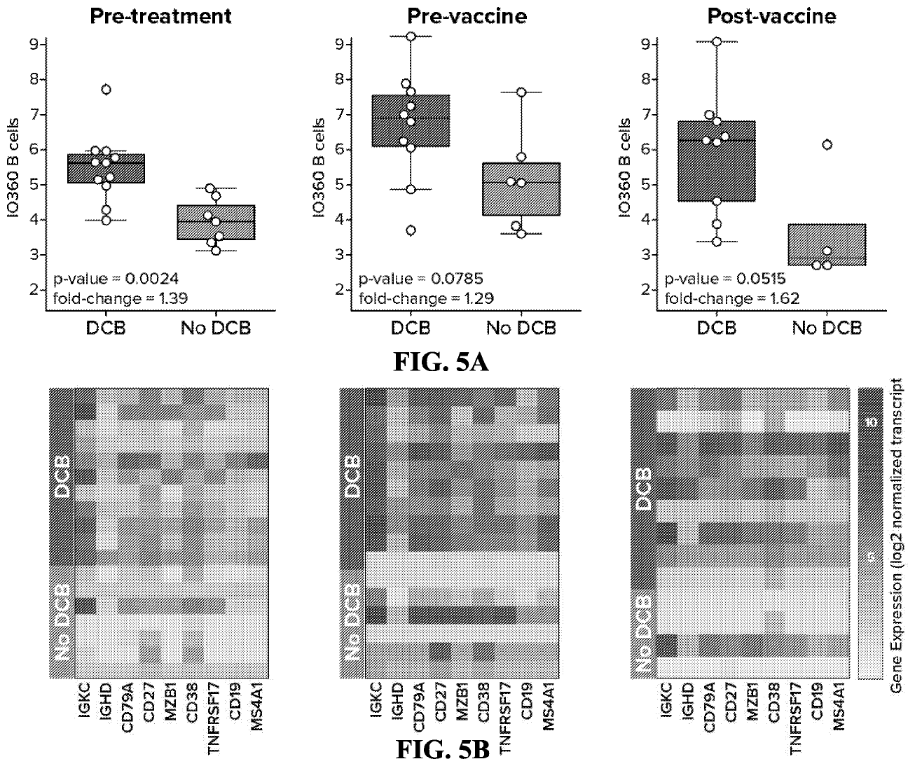

In a further assay, a B cell signature was compared between DCB and no-DCB melanoma patients prior to treatment (pre-treatment), after 12 weeks of nivolumab monotherapy (pre-vaccine), and after completion of NEO-PV-01+nivolumab vaccination (post-vaccine). Patients with DCB have higher B-cells signature and B cell gene expression (FIG. 5A).

Shown in FIG. 5B are genes associated with B cells, including IGKC, that were analyzed across all three timepoints at an individual patient level. Heatmap shows gene expression in a log 2 scale. B cell gene expression appears to be predictive of outcome. Patients that have higher B cell gene expression also have prolonged PFS. Expression of B cells genes also appears to be driven by treatment, with patients that have prolonged PFS have an increase in B cell gene expression after treatment. The presence of B cells was shown to be associated with improved patient outcome and is associated with tertiary lymphoid structures in tumors (with Example 5).

### Example 5. Genes Associated with Tertiary Lymphoid Structures (TLS) in TME Signature are Enhanced in Patients with DCB

TLS signature was investigated in biopsies prior to treatment (pre-treatment), after 12 weeks of nivolumab monotherapy (pre-vaccine), and after completion of NEO-PV-01+nivolumab vaccination (post-vaccine). as described earlier. Genes associated with tertiary lymphoid structure, including chemokines, cytokines, and cell types, were used to calculate the TLS signature.

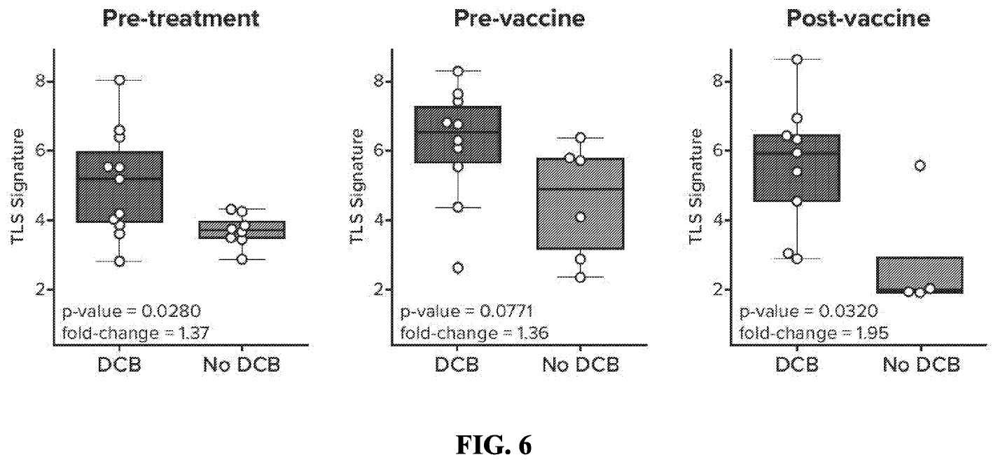

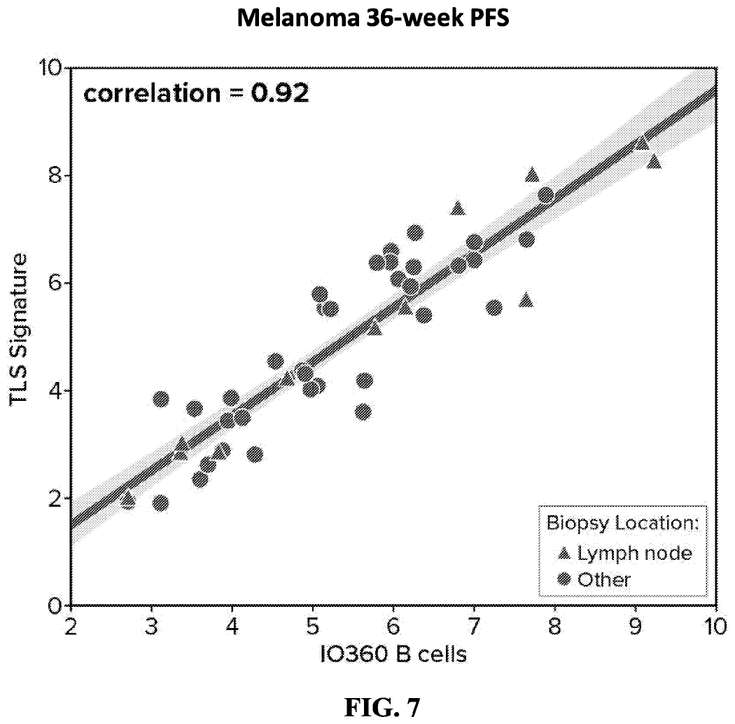

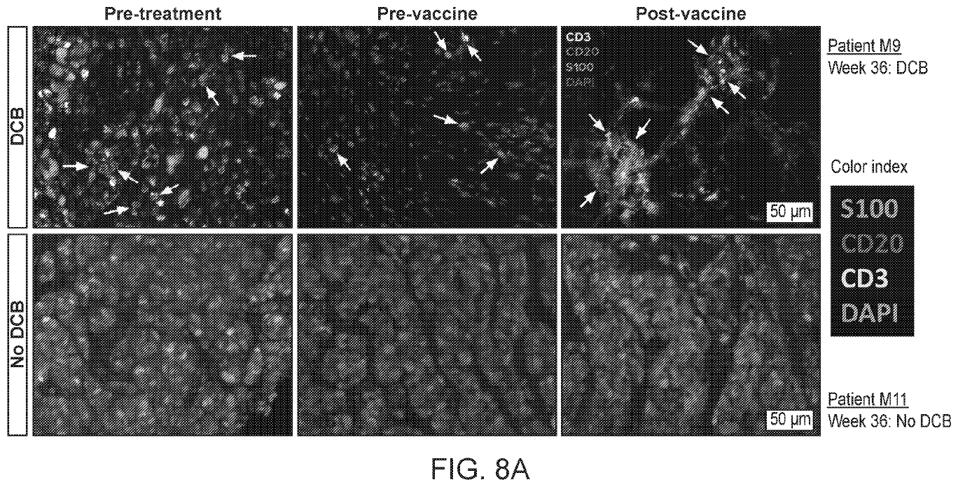

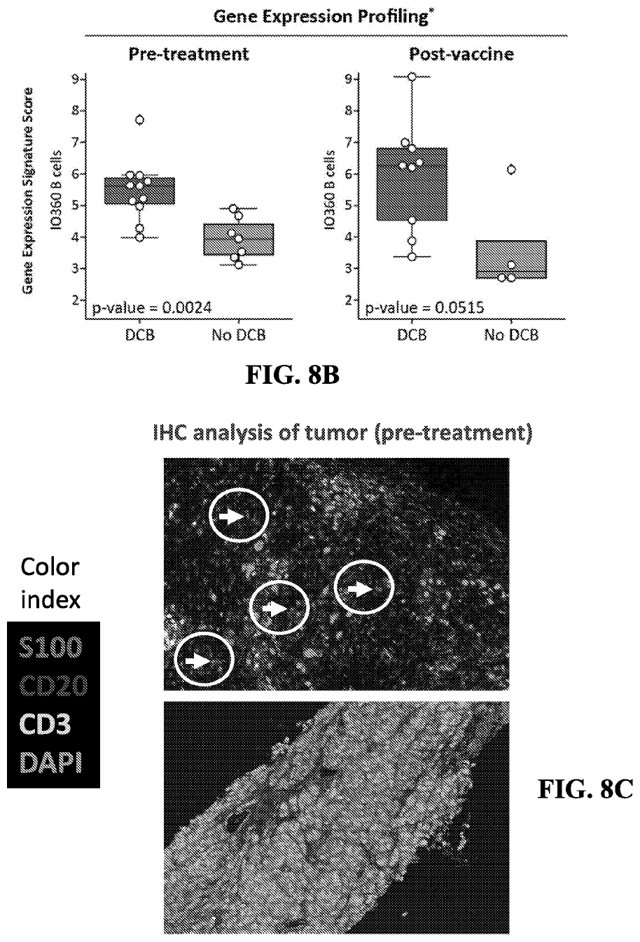

As shown in FIG. 6, Patients with DCB have increased expression of genes associated with the presence of tertiary lymphoid structures. In a comparative study, the TLS signature correlated well with the B cell signature (FIG. 7). A multiplexed immunohistochemical analysis (FIG. 8A, 8C) demonstrate the presence of B cell marker CD20+, T cell marker CD3+ cells, and tumor cells (S100), all of which were used simultaneously to examine the tertiary lymphoid structures in patients with DCB and no DCB. A representative patient from each cohort is shown in FIG. 8A. The presence of individual and clusters of B cells are denoted by white arrows, and T cells are indicated by yellow arrows (FIG. 8A). Additionally, FIGS. 5A, 8B and 8C show that there is a positive difference in the levels of these markers at pre-treatment between the subjects that showed DCB vs. no DCB, further demonstrating the predictive value of the markers.

### Example 6. Gene Expression Associated with Cytotoxic CD56dim NK Cells in TME Signature is Higher in Patients with DCB

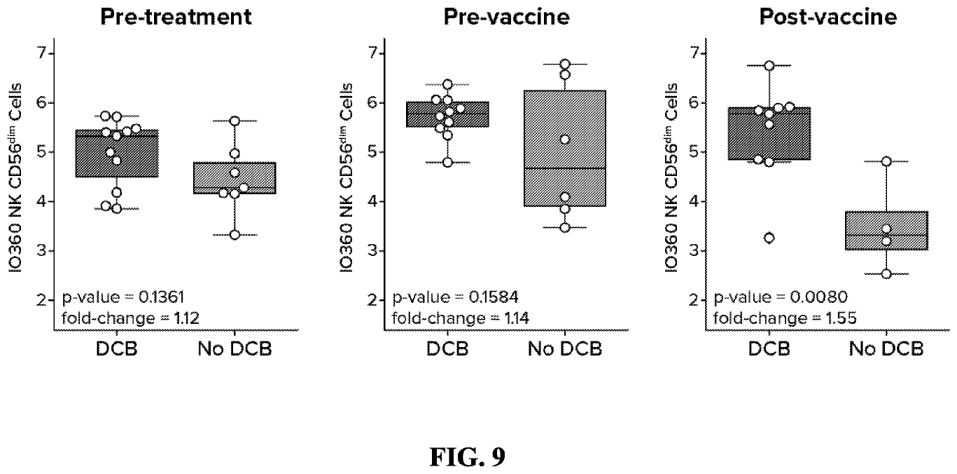

A representative NK cell signature was investigated in tumor biopsies prior to treatment (pre-treatment), after 12 weeks of nivolumab monotherapy (pre-vaccine), and after completion of NEO-PV-01+nivolumab vaccination (post-vaccine). Expression of genes associated with cytolytic CD56dim NK cells is increased in patients with DCB at the post-vaccine timepoint (FIG. 9). This data indicates a role of NK cells in the immune response within the TME.

### Example 7. MHC Class II Signature is Associated with DCB in Melanoma Patients

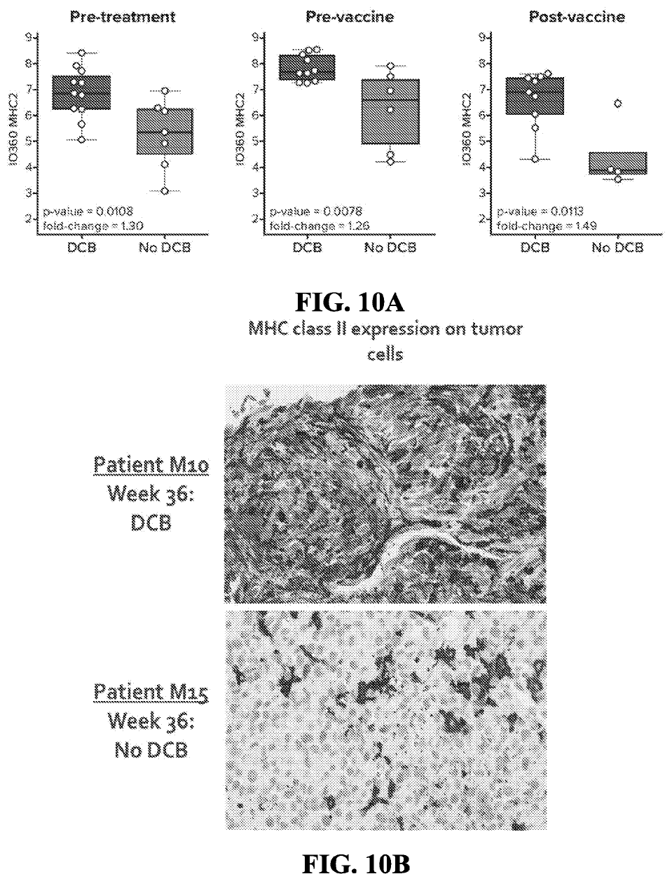

A representative MHC-II signature was investigated in tumor biopsies prior to treatment (pre-treatment), after 12 weeks of nivolumab monotherapy (pre-vaccine), and after completion of NEO-PV-01+nivolumab vaccination (post-vaccine). As shown in FIG. 10A, patients with DCB have higher expression of MHC class II indicating MHC class II gene expression at the pre-treatment timepoint is predictive of outcome and expression increases in the TME post-treatment.

Expression of MHC class II on professional antigen presenting cells could potentially lead to activation of CD4+ T cells and MHC class II expression on tumor cells would allow for recognition of these tumor cells by CD4+ T cells. On an immunohistochemical examination of MHCII expressing cells, striking difference was observed between representative DCB and no DCB tumor sample (FIG. 10B). MHC class II expression on tumor cells has been associated with therapeutic response and infiltration of CD4+ and CD8+ T cells in the tumor.

### Example 8. B7-113 Gene Expression in TME Signature is Increased in Melanoma Patients with No DCB

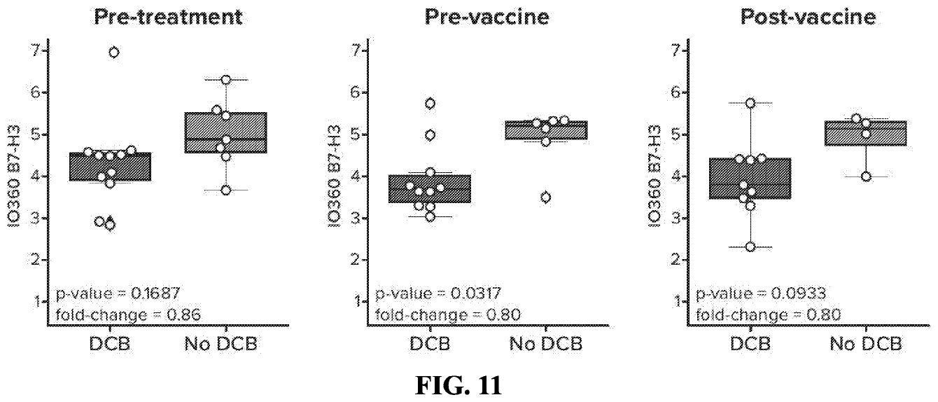

A representative B7-H3 gene signature was investigated in tumor biopsies prior to treatment (pre-treatment), after 12 weeks of nivolumab monotherapy (pre-vaccine), and after completion of NEO-PV-01+nivolumab vaccination (post-vaccine). As shown in FIG. 11, expression of the inhibitory ligand B7-H3 is higher in patients with no DCB. Overexpression of B7-H3 is known to contribute to immune suppression and is associated with poor prognosis.

### Example 9. Durable Clinical Benefit with NEO-PV-01 Vaccine

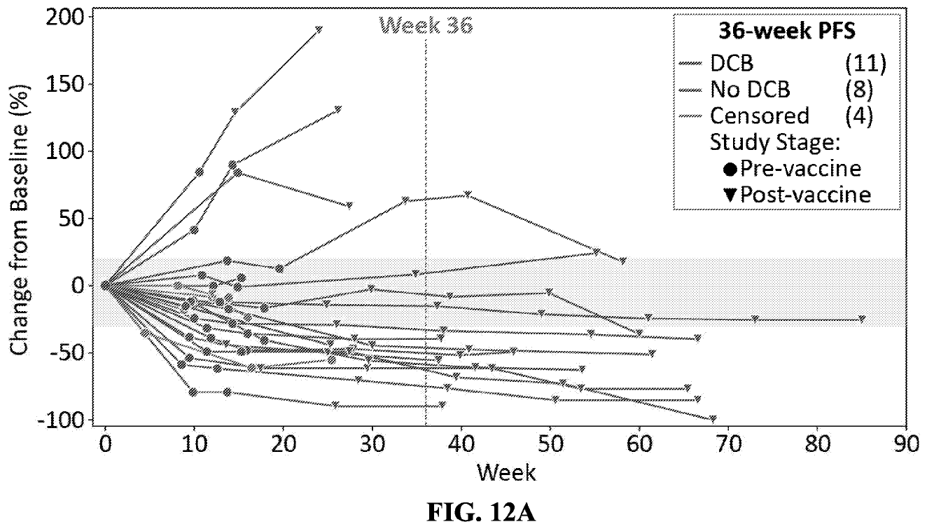

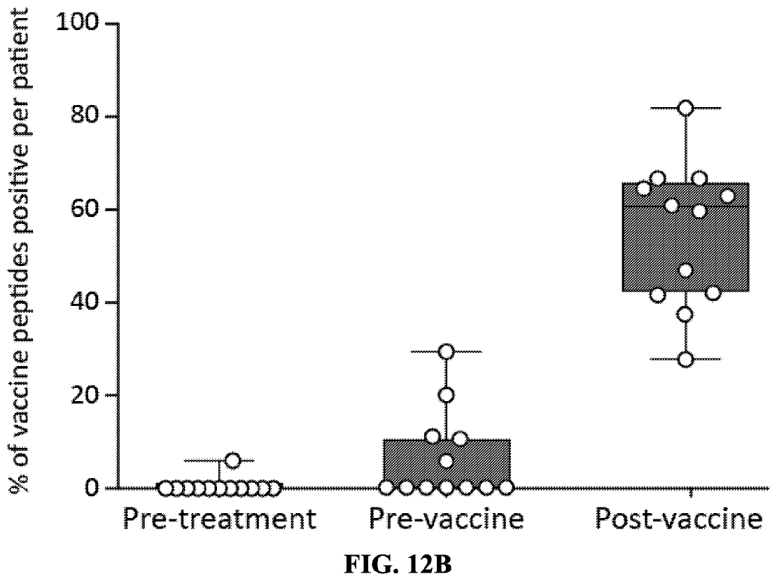

In this example, provided herein are the results of the NT-001 clinical trial, which demonstrate unexpectedly high DCB. Melanoma patients (n=23) demonstrated 36-week progression free survival (PFS) (FIG. 12A). However, in addition, several patients have progressed further, and show a PFS between 52-55 weeks. One patient is demonstrated to proceed to greater than 85 weeks. (FIG. 12A).

In an assessment of peptide specific response in NT-001 study, patients demonstrated positive for approximately 40-62% of vaccine peptides per person (FIG. 12B). Approximately 5-12 peptides generated immune response in a patient. It was found that about 55% of the epitopes generated at least a T cell response, as measured by IFN-γ ELISpot, about 42% of the epitopes generated a CD4 response, and about 28% of the epitopes generated a CD8 response. It was also observed that all patients were positive for measurable ex vivo immune responses. Durability of immune responses was observed at least up to 52 weeks in 4 out of 7 melanoma patients observed.

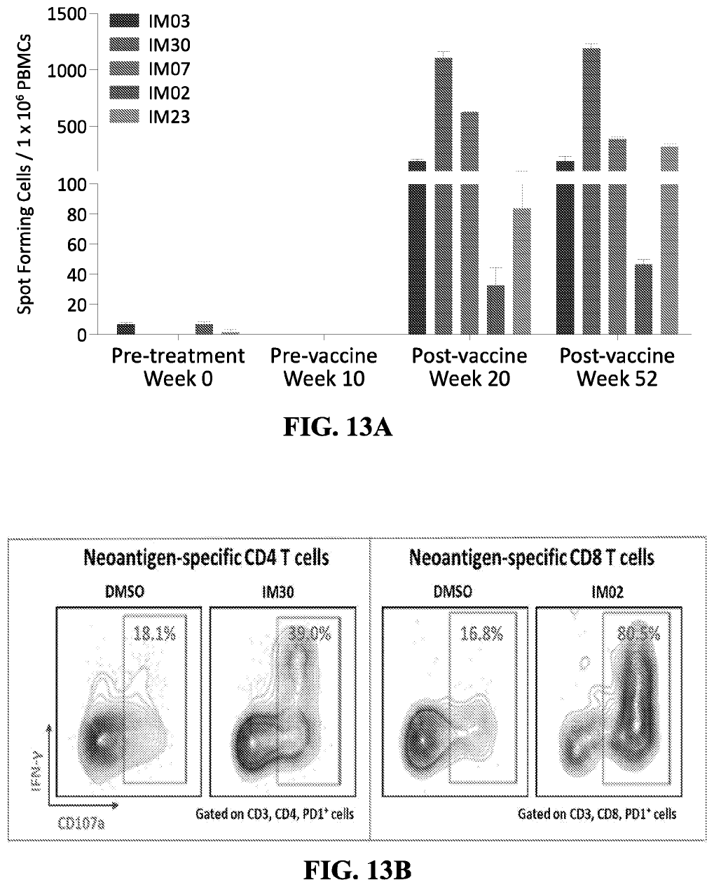

Immune responses were followed in one exemplary patient receiving nivolumab+Neo-PV-01 vaccine for assessment of DCB. It was observed that a 5 day exposure to 8 out of 17 immunizing peptides (IM) triggered a high IFN-γ response in the patient at 20 weeks and at 52 weeks post vaccination (FIG. 13A). Cytolytic and functional markers for neoantigen-specific CD4 and neoantigen-specific CD8 cells were evaluated (FIG. 13B) Gated on CD3, CD4 and PD1+ cells, it was observed that the neoantigen-specific cells expressed high levels of both IFN-γ and CD107a.

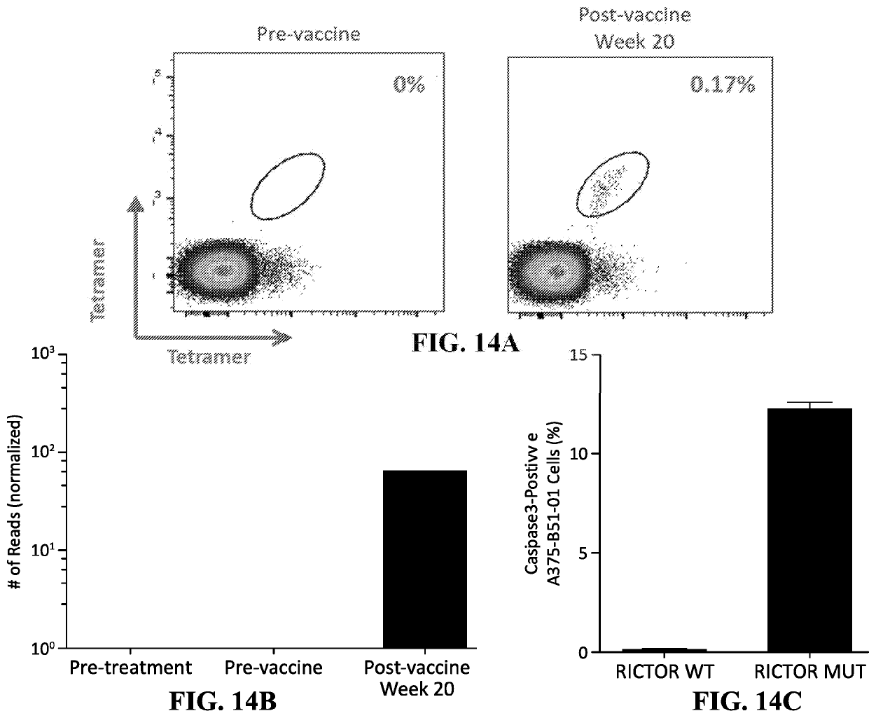

In a sample examination of a Neo-PV-01 vaccine treated patient, peptide tetramer specific CD8+ T cells were observed in the patient's blood at week 20 (FIG. 14A). Additionally, neo-antigen (corresponding to a mutated RICTOR epitope)-specific T cell receptor (TCR) was detected in the tumor, at 20 weeks post-vaccine (FIG. 14B). A375-B51-01 cells stimulated with PBMCs from a patient obtained at pre-treatment and transduced with RICTOR mutant-specific TCR showed high percentage of Caspase 3 activation indicating high activation and cytolytic potential of the neoantigen-specific TCR (FIG. 14C).

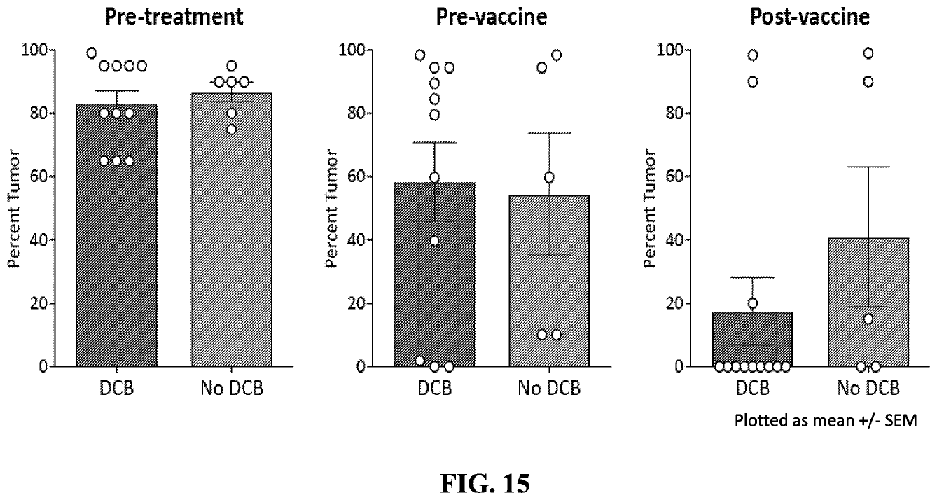

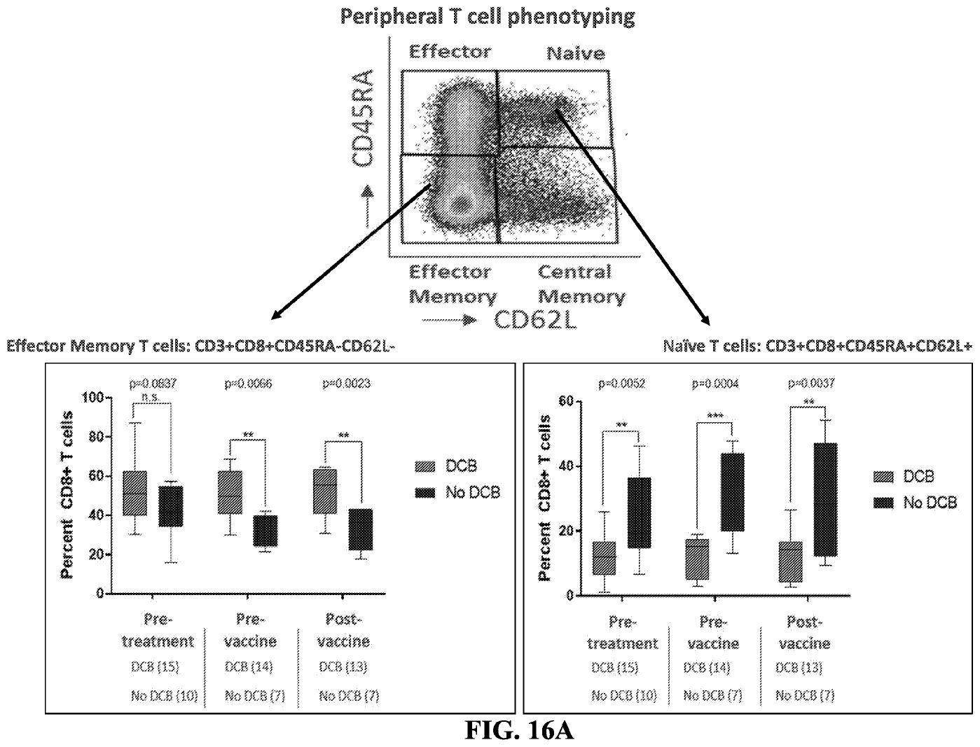

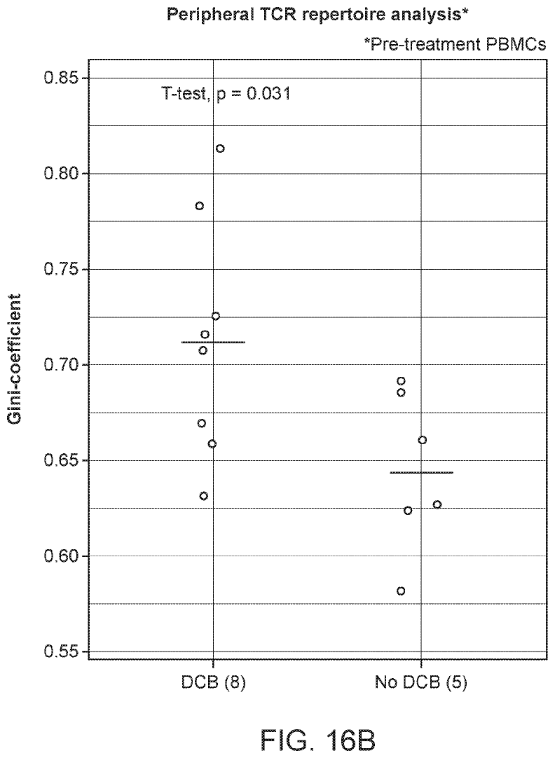

H&E analysis by independent pathology review from biopsies were analyzed at each time point. As shown in FIG. 15, the respective scores for DCB and No-DCB were indistinguishable in pretreatment samples. The pre-vaccine samples correspond to the histological evaluation of tumor in patients who have undergone 12 weeks of treatment with nivolumab. It was clear from examination of such patients, that even in the DCB patients with nivolumab treatment alone, tumor reduction was not appreciable (middle panel, FIG. 15). However, in post-vaccine group, the histology demonstrated high degree of tumor reduction (reduction to about 20%) in the vaccine treated patients, compared to approximately 40% or greater in the No-DCB patients. A minimum of 1-5 biopsies were obtained at each time points, and the results were expressed and mean+/−SEM.

These studies demonstrate that the Neoantigen specific vaccine induce specific DCB, which is long term, and with the ultimate read-out of high degree of tumor reduction in patients with DCB. Surprisingly, the treatment with specific neoantigen vaccines as described herein appear superior to nivolumab, a standard of care therapy for melanoma at the time of the study.

Additionally, it was clear that the markers for DCB described here strongly correlate with high degree of correlation with actual tumor reduction and pathophysiological remission of the disease.

### Example 10. Predictive Biomarkers for Treatment with NEO-PV-01 from the Analysis of Peripheral Blood Mononuclear Cells

This example illustrates, inter alia, identification of biomarkers from immune phenotyping of peripheral blood mononuclear cells (PBMCs). In addition, it shows that the identified biomarkers could be predictive biomarkers.

PBMC was isolated from patients enrolled in NT001 clinical trial for melanoma, lung and bladder patients enrolled in the NT001 study. Immune phenotyping was performed on the isolated cells using fluorescence activated cell sorting, and subsequent analysis on the FlowJo software. The biomarkers were trained on a subset of melanoma, lung and bladder patients enrolled in the NT001 study. These can be validated with (1) a subset of patients from the trial that are not used in training, and/or (2) patients in from subsequent clinical trials. The biomarkers can be used as an inclusion or exclusion criteria for future patient enrollment, and/or characterize a patient's molecular response over the course of treatment.

### Peripheral Sample Flow Cytometry Staining Protocol:

Patient PBMCs were thawed into FBS, followed by a wash with Lonza X-vivo media to remove cells from DMSO. Cells were then treated with benzonase for 30 minutes at a 1:1000 dilution in media at 37° C. Cells were washed with media and counted using the Guava easyCyte Flow cytometer. 2*10⁶ cells per sample were plated for flow staining and washed once with FACS buffer (PBS+0.5% BSA). Cells were then incubated with surface stain antibody cocktails for 30 minutes on ice, followed by a wash with FACS buffer. Next, cells were fixed and permeabilized for intracellular staining using one of two methods (depending on the panel) for 20 minutes on ice. All cells stained using the B cell and myeloid cell panels were fixed and permeabilized using the BD Cytofix/Cytoperm kit according the manufacturer's instructions. All cells stained with the T cell panel were fixed and permeabilized using the Invitrogen FOXP3 staining buffer set Fixation/Permeabilization concentrate and diluent according to the manufacturer's instructions. Cells were washed with the corresponding permeabilization wash buffer according to the manufacturer's instructions. Cells were then incubated with intracellular antibodies in the corresponding permeabilization wash buffer for 30 minutes on ice, washed with the appropriate permeabilization wash buffer, followed by a final wash with FACS buffer. Cells were stored in FACS buffer at 4° C. until run on a BD LSR Fortessa flow cytometer. Analysis was performed using FlowJo version 10.5.0. FIGS. 16Ii-ii show an exemplary gating strategy for flow cytometry of the indicated cells.

Naïve B Cells were gated as live, single cells that are CD56−, CD3−, CD14−, CD19+, IgD+ and CD27−. Plasmacytoid DCs (pDCs) were gated as live, single cells that are CD3−, CD19−, CD56−, CD14−, CD11c−, CD123+ and CD303+.

### Results:

Analysis of naïve T cells at pretreatment and at 20 weeks after therapy showed that subjects with a higher naïve CD8+ T cell signature at pretreatment is associated with poor outcome measured by DCB in melanoma, patients enrolled in the NT001 study (FIG. 16A).

PBMCs from melanoma patients from the three timepoints were immunophenotyped for naïve T cell markers as defined by the expression of the markers CD62L and CD45RA (FIG. 16A, top center panel). Patients who receive durable clinical benefit as defined by progression free survival 9 months post initiation of treatment had higher levels of effector memory T cells (FIG. 16A, bottom left panel) and lower levels of naïve T cells (FIG. 16B, right panel) across all three time points when compared to patients who progressed. The ratio of the number of naïve CD8+ T cells to total CD8+ T cells in the PBMCs of the peripheral blood sample from the subjects were determined by flow cytometry as described above. Subjects that demonstrated DCB upon treatment with either nivolumab alone or nivolumab with neoantigen vaccine had about 20% (20:100) or lower naïve CD8+:CD8+ T cell ratio at pretreatment. Additionally, irrespective of whether the treatment was nivolumab alone or nivolumab with neoantigen vaccine, lower naïve CD8+ T cell counts prior to treatment was associated with DCB, and conversely, higher naïve CD8+ T cell count at pretreatment was associated with no DCB. Percent CD8+ naïve T cells of less than 20% of the total CD8+ T cells in a peripheral blood sample at pretreatment is therefore associated with DCB as shown in FIG. 16A, bottom right panel).

Various features of the peripheral T cell receptor repertoire of patients were quantified to better understand the state of their immune system and how it relates to their response to the treatment. In this analysis, a coefficient called the “Gini Coefficient” was calculated in the pretreatment PBMCs of patients. It is a parameter of a distribution in a population using a number between 0 and 1, where 0 represents complete clonal type distribution and 1 represents a case in which one clonotype dominates the entire population. In this analysis, 0 represents a case where all T cell CDR3 amino acid clonotypes are found at the same frequency and 1 a case where one clone dominates the repertoire. The patient who had a durable clinical benefit had an increased Gini Coefficient compared with patients without durable clinical benefit, indicating that a more skewed frequency distribution of the repertoire is associated with response to treatment (FIG. 16B).

Low levels of naïve B cells in PBMC was associated with DCB (FIG. 16C).

Conversely, higher naïve B cell levels at pretreatment was associated with lack of DCB using two different therapeutic regimens, nivolumab alone or nivolumab with neoantigen vaccine. Ratio of the number of naïve B cells to total CD19+ cells (a pan B cells marker) in the PBMCs of the peripheral blood sample from the subjects were determined by flow cytometry as described above. A value of less than 70% (70:100) in this case determined at pretreatment was associated with DCB at 36 weeks.

PBMCs from melanoma patients from the three timepoints were immunophenotyped for class switched memory B cells as defined by the expression of the markers IgD and CD27 on CD19 positive B cells (FIG. 16D, top panel). Patients who receive durable clinical benefit as defined by progression free survival 9 months post initiation of treatment had higher levels of class switched memory B cells (FIG. 16D, bottom panel) across all three time points when compared to patients who progressed (No DCB).

More functional BCR Ig CDR3 sequences (in terms of both number of unique sequences and total number of CDR3 sequences observed) were observed in the tumor microenvironment at pretreatment time point in melanoma patients who receive durable clinical benefit from the therapeutic regimen compared to those who do not (FIG. 16E). These CDR3 sequences were reconstructed using MiXCR from short read RNA-seq data from pre-treatment tumor biopsies.

PBMCs from NSCLC patients from the three indicated timepoints were immunophenotyped for expression of plasmacytoid DC markers on Lin−/CD11c− cells (FIG. 16F, top panel). FIG. 16F shows that low levels of plasmacytoid dendritic cells (DCs) in PBMCs was associated with DCB. Conversely, higher plasmacytoid DCs in PBMCs was associated with lack of DCB using two different therapeutic regimens. As shown in the bottom panel of FIG. 16F, peripheral blood samples from subjects with DCB at 36 weeks have a ratio of plasmacytoid dendritic cells to total Lin−/CD11c− cells that is 3:100 or less or less than 3:100. With both nivolumab treatment or neoantigen vaccine in combination with nivolumab therapy, average plasmacytoid DCs of the no-DCB group showed a trend towards was mild reduction at 20 weeks compared to pretreatment, while the levels do not change substantially in the DCB subjects. This observation indicates that plasmacytoid DC levels may be affected by the treatments with immune checkpoint inhibitor, and a combination therapy with neoantigen vaccine, but nonetheless, a high level of plasmacytoid DCs at pretreatment is an indicator of poor treatment response.

PBMCs from NSCLC patients from the three indicated timepoints were immunophenotyped for expression of the immune suppressor markers CTLA4 on CD4 positive T cells (FIG. 16G, top panel). Patients who receive durable clinical benefit as defined by progression free survival 9 months post initiation of treatment had lower levels of CTLA4 on CD4 positive T cells (FIG. 16G, bottom panel) at the pretreatment time point when compared to patients who progressed (no DCB).

PBMCs from TCC of bladder patients from the three indicated timepoints were immunophenotyped for naïve and memory T cell markers as defined by the expression of the markers CD45RO and CD45RA (FIG. 16H, top panel). Patients who receive durable clinical benefit as defined by progression free survival 6 months post initiation of treatment had higher levels of memory T cells (FIG. 16H, bottom panel) when compared to patients who progressed specifically in the post vaccine time point. This marker could be used as mechanistic marker for evaluating vaccine effect post treatment.

The results discussed above indicate that a treatment outcome on a subject can be predicted by performing a quantitative analysis of these cell types at pretreatment. It is also possible to infer the outcome based on the cell percentages, because of the clear distinction in percentages of each cell types between DCB and no-DCB patients.

Other parameters are likewise being evaluated for peripheral blood signatures of DCB. These include but are not limited to:

(a) CD4:CD8 T cell ratio,
(b) proportions of effector memory T cells and naïve CD4 and CD8 T cell subsets,
(c) proportion of T regulatory cells,
(d) T cell PD1 expression,
(e) T cell CTLA-4 expression,
(f) proportions of gamma-delta T cells,
(g) proportions of CD11b+ CD33+ myeloid cells,
(h) proportions of monocytes,
(i) proportions of CD11c+ DCs,

### (j) CD141+ CLEC9A+DCs,

(k) proportions of plasmacytoid DCs,
(1) proportions of NK cells (including activation/inhibitory receptor expression and Perforin/Granzyme B expression), and
(m) proportions of B cells.

### Example 11. ApoE Variants in a Melanoma Cohort Treated with Nivolumab and Neoantigenic Peptides

ApoE variants associate with size of the lesion in melanoma cohort of an ongoing clinical trial with nivolumab in combination with neoantigenic peptides. As shown in FIG. 17, subjects are categorized on the basis of whether they are ApoE2 heterozygous, ApoE4 heterozygous, ApoE4 homozygous, or ApoE3 homozygous. ApoE3 homozygous allele is the reference allele. Each line plot represents the % change in the sum of target lesions, with increase in lesions shown as values above baseline, and decrease in lesions shown below the baseline. From the above, ApoE4 is found to be a protective variant, and subjects that are homo- or heterozygous for the ApoE4 variant respond positively to the nivolumab+neoantigenic peptides over time as measured from their baseline tumor lesion sizes or changes in lesion sizes over the course of therapy. Similar studies are ongoing in lung and bladder cancer cohorts.

### Example 12: ApoE Variants in a Melanoma Cohort Treated with Pembrolizumab Alone

In this exemplary study, data from a clinical trial involving pembrolizumab (anti-PD1 therapy, checkpoint inhibitor) melanoma cohort were reanalyzed for evaluation of ApoE protective variants (Hugo et al., 2016, Cell 165, 35-44). In this study, subjects were treated with checkpoint inhibitor pembrolizumab. As shown in the data presented in Table 3, none of the ApoE genetic variants show a specific correlation with treatment outcome when the therapeutic agent is anti-PD1 monotherapy.

TABLE 3
Patient genotype and drug responsiveness to Pembrolizumab

| | Anti-PD1 | | | | | |
|---|---|---|---|---|---|---|
| Patient # | Response | Disease Status | Gender | Tissue | Treatment | Genotype |
| 1 | Progressive | M1b | Female | Melanoma | Pembrolizumab | E4 het |
| | Disease | | | Biopsies | | |
| 2 | Progressive | M1a | Male | Melanoma | Pembrolizumab | E3 |
| | Disease | | | Biopsies | | |
| 3 | Progressive | M1c | Male | Melanoma | Pembrolizumab | E3 |
| | Disease | | | Biopsies | | |
| 4 | Complete | M1c | Female | Melanoma | Pembrolizumab | E3 |
| | Response | | | Biopsies | | |
| 5 | Progressive | M1c | Female | Melanoma | Pembrolizumab | E3 |
| | Disease | | | Biopsies | | |
| 6 | Partial | M1b | Male | Melanoma | Pembrolizumab | E3 |
| | Response | | | Biopsies | | |
| 7 | Progressive | M1b | Male | Melanoma | Pembrolizumab | E4 het |
| | Disease | | | Biopsies | | |
| 8 | Partial | M1c | Male | Melanoma | Pembrolizumab | E4 het |
| | Response | | | Biopsies | | |
| 9 | Partial | M1c | Male | Melanoma | Pembrolizumab | E3 |
| | Response | | | Biopsies | | |
| 10 | Progressive | M1c | Female | Melanoma | Pembrolizumab | E3 |
| | Disease | | | Biopsies | | |
| 11 | Progressive | M1c | Male | Melanoma | Pembrolizumab | E4 het |
| | Disease | | | Biopsies | | |
| 12 | Progressive | M1c | Male | Melanoma | Pembrolizumab | E3 |
| | Disease | | | Biopsies | | |
| 13 | Progressive | M1c | Male | Melanoma | Pembrolizumab | E4 het |
| | Disease | | | Biopsies | | |
| 14 | Complete | M1c | Male | Melanoma | Pembrolizumab | E3 |
| | Response | | | Biopsies | | |
| 15 | Partial | M1c | Male | Melanoma | Pembrolizumab | E3 |
| | Response | | | Biopsies | | |
| 16 | Progressive | M1c | Male | Melanoma | Pembrolizumab | E3 |
| | Disease | | | Biopsies | | |
| 17 | Progressive | M1c | Male | Melanoma | Pembrolizumab | E3 |
| | Disease | | | Biopsies | | |
| 18 | Progressive | M1c | Male | Melanoma | Pembrolizumab | E3 |
| | Disease | | | Biopsies | | |
| 19 | Partial | M1c | Female | Melanoma | Pembrolizumab | E3 |
| | Response | | | Biopsies | | |
| 20 | Partial | M1c | Female | Melanoma | Pembrolizumab | E3 |
| | Response | | | Biopsies | | |
| 21 | Partial | M1c | Female | Melanoma | Pembrolizumab | E3 |
| | Response | | | Biopsies | | |
| 22 | Partial | M1c | Male | Melanoma | Pembrolizumab | E3 |
| | Response | | | Biopsies | | |
| 23 | Partial | M1c | Male | Melanoma | Pembrolizumab | E4 Het |
| | Response | | | Biopsies | | |
| 24 | Partial | M1c | Male | Melanoma | Pembrolizumab | E3 |
| | Response | | | Biopsies | | |
| 25 | Progressive | M1c | Female | Melanoma | Pembrolizumab | E4 |
| | Disease | | | Biopsies | | |
| 26 | Complete | M1a | Male | Melanoma | Pembrolizumab | E3 |
| | Response | | | Biopsies | | |
| 27 | Complete | M0 | Male | Melanoma | Pembrolizumab | E2 |
| | Response | | | Biopsies | | |

### Example 13: TCR Repertoire Profiling and DCB

To assess whether comprehensive peripheral analysis conveys predictive power of melanoma patients' responses to personalized neo-antigen cancer vaccine (NEO-PV-01) combined with nivolumab in the NT-001 clinical trial (NCT02897765), the TCR repertoire features of patients and frequencies of immune cell subpopulations were analyzed.

Patients enrolled in the melanoma cohort of the neoantigen vaccine trial NT-001 (NCT02897765) received nivolumab combined with the personalized neoantigen vaccine NEO-PV-01 (FIG. 18). Three leukapheresis samples were taken at week 0 (preT=Pretreatment (Week 0 pre-Nivolumab)), week 10 (preV=pre-vaccine administration), and week 20 (postV=post-vaccine administration).

TCR repertoires were generated by running a licensed copy of MiXCR (version 3.0.12) on the paired-end raw sequencing fastq files. The parameters included the species specifications (Human, hsa), starting material (RNA), 5′ and 3′ primers (v and c primers, respectively) with no adapters, and searching for TCRβ chains (trb).

TCRβ CDR3 clonotypes were filtered by removal of non-functional sequences (out-of-frame sequences or those containing stop codons). Clonal frequency was calculated based on the clonal count for each clone out of the total count.

Analysis of Peripheral Blood Samples:

Isolated T cell RNA was subjected to arm-PCR targeted to the TCR beta chain locus and TCR sequencing. 65 samples from 21 patients were analyzed for clonal composition characteristic of TCR repertoires. To test for the skewedness of the frequency distributions, datasets of TCR identities and frequencies were tested for repertoire-wide clonality parameters at each time point. DE50, Gini coefficient, Shannon's entropy, Lorentz curves, and the number of unique nucleotide and amino-acid complementarity determining region 3 (CDR3) were calculated to test the association of TCR identities and frequencies with DCB status (FIGS. 19A and 19B).

TCR Repertoire Diversity/Clonality Analysis:

Clone size-designation (FIG. 20A, FIG. 20B, and FIG. 20C) was based on clonal frequency, Fi as follows: rare (Fi<1e-6), small (1e-6≤Fi<1e-5), medium (1e-5≤Fi<1e-4), large (1e-4≤Fi<1e-3), and hyperexpanded (1e-3≤Fi). The unique number of nucleotide (nt)/amino acid (aa) TCRβ CDR3s was calculated per sample. Global diversity/clonality coefficients have been calculated as follows:
DE50—aa CDR3 clonotypes were sorted based on their frequency in descending order. The cumulative frequencies of this sorted frequency vector were calculated. The rank of the first value that was equal or larger than 0.50 was divided by the total number of unique aa CDR3 clonotypes to obtain the DE50 value. For example, if the 40 most frequent clones (but not 39) of a repertoire covers 50% of the total counts of the clones in that repertoire, consisting of 1000 clones, the DE50 value would be 0.04.
Gini Coefficient—ranges between 0 (all clones are equally frequent—repertoire diversity) and 1 (frequency dominated by one clone, repertoire clonality). Calculated using the “Gini” function from the “DescTools” R package.
Shannon's Entropy—higher values represent higher inequality of the frequencies. Calculated using the “Entropy” function from the “DescTools” R package.
Lorentz curves—similarly to the DE50 estimate, but a continuous curve between DE0 and DE100. Calculated using the “Lc” function from the “DescTools” R package.
Sum of squares—the sum of squares measurement is calculated as the sum of the frequencies of the aa CDR3 clonotypes, each squared.

These parameters indicated an increased clonality of the peripheral T cell repertoire in DCB patients at all three time points. Similar comparisons of TCR repertoire parameters with patient's age, sex, TMB etc. showed no correlation (data not shown). Taken together, these data indicated that peripheral TCR repertoire clonality of NT-001 melanoma patients is increased in DCB patients, even prior to initiation of treatment, and may serve as a minimally invasive biomarker for treatment success. To establish significance, the fraction of clones in each size-designation/category of DCB with no DCB patients individually at each time point were compared (FIGS. 20A, 20B, and 20C). Interestingly, at preT=Pretreatment (Week 0 pre-Nivolumab) and preV=pre-vaccine administration) each size-designation/category appears to represent a significant predictor of DCB status, whereas at postV=post-vaccine administration, only the hyperexpanded category shows a significant difference. These results indicate that patients with DCB have an increased proportion of larger clones at the expense of smaller clones and are especially enriched for hyperexpanded clones. Furthermore, similar differences were detected between HD and patients with DCB, but not with patients without DCB.

Analysis of Lorenz curve (FIGS. 21A and 21B) show distinct trend towards higher inequality of CDR3 sequences in the DCB, and not in the No-DCB patient samples.

Turn-over rates were tested, as measured by the Jensen Shannon Divergence (JSD, FIG. 22A), and results show that turnover rates also correlated with DCB status (FIG. 22B). Analyzing the most frequent clones (covering the top 20% of the repertoire) in each repertoire, the JSD of the preV (pre-vaccine administration) and postV (post-vaccination) time points were measured, both in comparison to preT=Pretreatment (Week 0 pre-Nivolumab). Both comparisons demonstrated significantly lower JSD values in DCB patients indicating lower turn-over rates of T cell clones. Results for extended time period of observation in some patients are shown (FIG. 22C). This difference remains significant regardless of the fraction of the repertoire used for the calculation. Of note, the repertoire of DCB patients remain stable not only between the preT=Pretreatment (Week 0 pre-Nivolumab) and preV (pre-vaccination) time points, but also between preT=Pretreatment (Week 0 pre-Nivolumab) and postV (post-vaccination), whereas the repertoire of no-DCB patients continues to change.

To further characterize repertoire stability, overlap across all three time points were tested using a Venn diagram as depicted in FIG. 23A. The cumulative frequencies of clones detected in only one time-point (A,B,C) is shown in FIG. 23C, two time-points (D,E,F) in FIG. 23D, and persistent clones found in all three samples (segment G) in FIG. 23B. This analysis showed that the cumulative frequency of persistent T cell clones (in segment G) is significantly increased in DCB patients (FIG. 23B), at the expense of clones detected in only one time-point (segments A, B, C, FIG. 23C). Importantly, no significant difference was detected in the number of unique clones in segment G between DCB patients and no-DCB patients (FIG. 23F).

The cumulative frequency of persisting clones (segment G), is increased in DCB patients due to having larger clones, and not more clones. This was further confirmed by analysis of the unique amino acids in DCB and No DCB clones (FIG. 23F).

The discrepancy between similar numbers of unique persistent clones and these clones having different cumulative frequencies, comparing DCB with no-DCB patients, points to differences in repertoire clonality. To test this hypothesis directly, the association between the Gini Coefficient and the cumulative frequency of the persistent clones were tested. A strong positive correlation with the cumulative frequency of the segment G clones was found (FIG. 23G), which indicates that repertoire clonality and repertoire stability are linked. The trend reverses when comparing the TCR clonality (Gini Coefficient) with the cumulative frequency of clones which were only detected at one time-point.

The cumulative frequency of the segment G clones with the frequency of immune cell sub-populations in peripheral blood mononuclear cells (PBMCs) were compared. Flow cytometry was used to phenotype our PBMCs, focusing on T and B cell populations. A strong positive correlation was found across patients between the cumulative frequency of the segment G clones and the frequency of effector-memory/memory CD8+ and CD4+ T cells, and the reverse trend with naïve T cell compartments (FIG. 2311). The data indicate that memory or effector-memory phenotypes of CD8, CD4 and B cells correlate with increased stability, while the reverse is true for naïve phenotypes. The ability to glean insights about B cell phenotypes from TCRβ CDR3 sequencing promotes viewing the state of our patients' immune system as a whole. Additional systemic measurements were taken including differences between clinical laboratory results from DCB and No DCB patients, including liver, and kidney function assays (ALT-SGPT, AST-SGOT, Creatinine), hemoglobin concentration and red blood cell (RBC) counts (FIG. 24A, top), and additional chemistry panels (Glucose, Potassium, etc.). Some of these measurements strongly correlated with the clonality and the stability of the TCR repertoire (FIG. 24A bottom). These findings further supported the idea that the state of the immune system of these melanoma patients expressed in a multitude of measurable avenues. Over 40 features measured from each patient at all three time-points of the trial were accumulated, including TCRβ sequencing clonality features, phenotyping of peripheral CD4 and CD8 T cells and B cells, and clinical laboratory results. Next, it was examined whether the measurements taken at baseline (pre-treatment) will be able to predict DCB. To reduce the dimensionality of all these features and distill the signal from them, principal component analysis (PCA), an unsupervised dimensionality reduction algorithm which seeks to represent the data along their axes with the strongest variance was used. Select measurements were taken at baseline from either the TCR repertoire analysis, the immunophenotyping of the PBMCs, or the clinical lab results were aggregated in one matrix. The matrix was centered and scaled, and PCA was calculated using the R function “prcomp” from the “stats” R package. The loadings, or contributions of the different measurements to PC1, were retrieved from the rotation matrix (FIG. 24D). Kaplan-Meier analysis was performed based on categorizing patients as belonging to PC1<0 or PC1>0. Calculation was performed using the “survfit” function from the “survival” R package and plotted using the “ggsurvplot” function from the “survminer” R package. P-value was calculated using the log-ratio test and hazard-ration calculated using a univariate Cox proportional hazards regression model. This analysis was performed in multiple approaches, each including a different set of peripheral measurements taken at baseline.

The algorithm was run with all the baseline features measured from our patients. Importantly, the algorithm was not provided the labels for the clinical status of DCB/No DCB patients. When plotting the patients along the two most significant axes of the reduced-dimensions (PC1 and PC2), it was clear that the algorithm separates DCB and No DCB patients along PC1 (FIG. 24B), (Table 4A and 4B). The fraction of clones in each patient which are shared with all 11 healthy donors (HD) were plotted against their PC1 scores (FIG. 24C). Clones shared with all 11 HD were defined as public clones, and the proportion of these clones out of the repertoire was defined as publicness. Patients with increased publicness have significantly lower PC1 values.

A Kaplan-Meyer curves for PFS of patients with PC1<0 (stemmed arrow) versus patients with PC1>0 (blunt arrow) (FIG. 25). Significant improvement was seen in PFS for PC1-positive patients.

### Analysis of Tumor Samples:

Tumor biopsy samples were analyzed from patients, using RNA as source material, either using iRepertoire targeted TCR assay or Personalis RNAseq of pretreatment and MiXCR sequencing analysis. Results shown in FIG. 26 indicate unique amino acid containing CDR3/TCR counts from tumor. It does not indicate that there were more detected clones in the DCB patient samples.

Number of clones shared between the MiXCR personalis RNA-seq clone detection and iRep peripheral blood repertoires were analyzed by Venn-diagram regions. Segment G seems to have the most amount of overlap (FIG. 27).

FIG. 28 shows data from tracking tumor clone frequencies in the tumor periphery. Each line represents data from one patient.

To summarize, significantly higher levels of TCR repertoire clonality and stability in DCB patients compared with no-DCB patients were detected and strong positive correlations of these features with T cell memory phenotypes. In addition, it was surprising that the same was found for B cell memory phenotypes. Principal component analysis (PCA) of analyzed features resulted in a strong predictive power that allowed us to determine DCB status from pre-treatment data. Overall, these results indicate that several peripheral features important for treatment success are correlated even across the T cell and B cell compartments, potentially pointing at an underlying, inherent immune health state that discriminates between DCB and no-DCB patients.

TABLE 4A
PCA table

| | Source of value: Flow Cytometry—T cell panel | | | | | | | | |
|---|---|---|---|---|---|---|---|---|---|
| | | | | Mem | NE | CM | EM | Naive | CM |
| patient | CD3 | CD4 | CD8 | CD8 | CD8 | CD8 | CD8 | CD8 | CD4 |
| M3 | 57.6 | 83.2 | 13.9 | 34.6 | 57.6 | 7.3 | 35.3 | 39.6 | 12.1 |
| M4 | 47.4 | 70.4 | 19.5 | 13.2 | 80.3 | 3.85 | 16.1 | 35.4 | 13.1 |
| M6 | 50 | 69.8 | 21.3 | 35.4 | 35.2 | 7.43 | 58.8 | 6.51 | 15.1 |
| M9 | 69.1 | 65.2 | 30.9 | 35.4 | 41.4 | 15.9 | 43.8 | 17.8 | 32.9 |
| M12 | 46.8 | 72.3 | 24.3 | 53.8 | 27.2 | 16.9 | 56.2 | 18.5 | 41 |
| M15 | 54.2 | 81.9 | 14.5 | 31.6 | 36 | 19.7 | 45.9 | 16.8 | 32.3 |
| M20 | 51.3 | 79.5 | 15 | 29 | 34.1 | 13.2 | 54.2 | 17.9 | 38.7 |
| M7 | 34 | 59.4 | 28 | 43.3 | 8.42 | 6.4 | 87.1 | 0.97 | 5.26 |
| M13 | 59.4 | 63.7 | 26.3 | 21.1 | 64.7 | 5.81 | 30.5 | 13.3 | 18 |
| M2 | 57.3 | 58.8 | 35.2 | 57.5 | 23.1 | 17.3 | 61.3 | 4.24 | 21.2 |
| M5 | 50.4 | 67.7 | 27.9 | 37.3 | 57.4 | 8.24 | 33.5 | 16.7 | 29.7 |
| M8 | 40.1 | 64 | 23.4 | 53.1 | 21.5 | 6.06 | 72.5 | 14.3 | 25.8 |
| M14 | 50.6 | 59.8 | 33.9 | 28.4 | 47.5 | 5.95 | 47.9 | 11.6 | 6.9 |
| M18 | 53.8 | 69.9 | 20 | 42.6 | 45.5 | 4.97 | 48.7 | 7.63 | 7.25 |
| M22 | 70.5 | 61 | 31.2 | 37.4 | 44.4 | 5.99 | 47.8 | 17.4 | 8.09 |
| M23 | 73.3 | 63.1 | 30.2 | 54.9 | 26.8 | 1.74 | 66.3 | 1.58 | 0.89 |
| M1 | 68.6 | 79.2 | 14.8 | 28 | 53.9 | 9.58 | 35.9 | 25.9 | 26.2 |
| M17 | 45.3 | 65.4 | 24.1 | 25.6 | 65.3 | 3.79 | 31.3 | 30.5 | 14.3 |
| | Source of value: Flow Cytometry—T cell panel | | | | | | | | |
| | EM | Naive | Mem | NE | CD8 + | CD4 + | CD8 + | CD4 + | CD3 + |
| patient | CD4 | CD4 | CD4 | CD4 | PD1+ | PD1+ | CTLA4+ | CTLA4+ | PD1+ |
| M3 | 41.3 | 26.2 | 56 | 43.8 | 17.9 | 8.9 | 0.74 | 3.61 | 14.5 |
| M4 | 52.2 | 17.3 | 66.3 | 33 | 5.05 | 6.26 | 0.65 | 4.23 | 9.94 |
| M6 | 67.6 | 8.57 | 85.1 | 14.6 | 9.96 | 7.79 | 0.32 | 4.9 | 12.5 |
| M9 | 27.9 | 35.4 | 62.4 | 37.7 | 14.4 | 5.63 | 0.3 | 6 | 12.4 |
| M12 | 31.9 | 22.8 | 66.7 | 32.8 | 20.9 | 5.76 | 0.75 | 6.5 | 13.8 |
| M15 | 31.6 | 28.6 | 57.6 | 41.4 | 0.45 | 0.27 | 1.03 | 7.28 | 1.03 |
| M20 | 30.5 | 25.2 | 69.6 | 30.1 | 10.4 | 4.04 | 0.69 | 6.86 | 9.51 |
| M7 | 87.1 | 0.25 | 95.3 | 0.61 | 22.8 | 27.1 | 0.04 | 0.23 | 36.7 |
| M13 | 65.2 | 6.15 | 85.8 | 14.1 | 12.1 | 9.14 | 0.76 | 4.98 | 17.9 |
| M2 | 53.7 | 14.5 | 72.5 | 26.8 | 20.9 | 9.99 | 0.42 | 6.97 | 18.6 |
| M5 | 57.6 | 8.77 | 89.3 | 10.4 | 1.96 | 0.66 | 0.6 | 6.37 | 2.91 |
| M8 | 67.3 | 4.18 | 94.3 | 5.15 | 20.3 | 7.38 | 1.04 | 8.72 | 16.6 |
| M14 | 58.4 | 6.08 | 67.2 | 32.5 | 3.03 | 4 | 0.44 | 6.41 | 5.96 |
| M18 | 73.5 | 1.74 | 83.2 | 16.5 | 20.5 | 13.4 | 0.57 | 5.76 | 21.4 |
| M22 | 68.8 | 4.36 | 78.7 | 20.9 | 18.5 | 11.1 | 0.48 | 5.05 | 18.9 |
| M23 | 79.2 | 0.45 | 86.5 | 13.3 | 18.6 | 8.94 | 0.33 | 3.53 | 17 |
| M1 | 34.3 | 24.5 | 65.7 | 33.9 | 15.7 | 11 | 0.53 | 5.35 | 18.4 |
| M17 | 36.2 | 30.2 | 52.1 | 47.6 | 20.6 | 11.3 | 0.67 | 6.51 | 17.7 |
| | Flow Cyto—B cell panel | | | | | | | | |
| | CS_ | | | TCR Features | | | | | |
| | mem | Naive | Transitional | Gini | Shannon | | | cdr3 | sumOfSq |
| patient | B % | B_% | B_% | Coeff | Emt | unqAA | DE50 | Length | uares |
| M3 | 9.36 | 77.2 | 12.1 | 0.5818 | 16.5 | 221908 | 0.1098 | 14.291 | 0.000092 |
| M4 | 3.13 | 82.2 | 3.26 | 0.6268 | 16 | 156304 | 0.101 | 14.248 | 0.000063 |
| M6 | 10.3 | 63.1 | 2.23 | 0.7254 | 13.1 | 37026 | 0.0566 | 14.201 | 0.001065 |
| M9 | 8.14 | 82.4 | 3.5 | 0.7127 | 14.7 | 115918 | 0.0649 | 14.282 | 0.002029 |
| M12 | 21.2 | 62.6 | 2.59 | 0.6691 | 15.9 | 197380 | 0.0743 | 14.306 | 0.000181 |
| M15 | 12.9 | 72 | 3.56 | 0.6604 | 16 | 208586 | 0.0794 | 14.229 | 0.000376 |
| M20 | 4.99 | 84.2 | 5.03 | 0.6915 | 15.3 | 137215 | 0.0712 | 14.239 | 0.000337 |
| M7 | 7.68 | 78.2 | 3.46 | 0.8365 | 10.7 | 49624 | 0.0084 | 14.372 | 0.033006 |
| M13 | 36.8 | 11.2 | 2.19 | 0.8134 | 11.5 | 52350 | 0.0114 | 14.407 | 0.016289 |
| M2 | 16.7 | 50.6 | 0.8 | 0.6313 | 16 | 189476 | 0.0904 | 14.29 | 0.00029 |
| M5 | 15.7 | 50.6 | 1.69 | 0.7709 | 12.9 | 42689 | 0.0462 | 14.134 | 0.003686 |
| M8 | 6.82 | 82.4 | 7.57 | 0.7823 | 12.5 | 27259 | 0.0465 | 14.3 | 0.000966 |
| M14 | 6.11 | 68.1 | 3.16 | 0.783 | 12.3 | 76767 | 0.0216 | 14.304 | 0.013668 |
| M18 | 14.4 | 69.6 | 3.25 | 0.7492 | 13.6 | 147874 | 0.0325 | 14.532 | 0.013974 |
| M22 | 28.5 | 49.9 | 3.07 | 0.7075 | 13.1 | 30199 | 0.083 | 14.234 | 0.000895 |
| M23 | 17.7 | 69.9 | 3.05 | 0.7477 | 14.5 | 162039 | 0.0325 | 14.481 | 0.001017 |
| M1 | 3.57 | 87.1 | 5.09 | 0.6586 | 15.6 | 165122 | 0.0777 | 14.32 | 0.000531 |
| M17 | 9.96 | 80.3 | 15.3 | 0.7004 | 15.3 | 166844 | 0.0677 | 14.694 | 0.000524 |

What is claimed is:

1. A method of treating a patient having a tumor comprising:
(a) determining if a sample collected from the patient is positive or negative for a biomarker which predicts that the patient is likely to have an anti-tumor response to a first therapeutic agent comprising (i) a one or more peptides comprising a neoepitope of a protein, (ii) a polynucleotide encoding the one or more peptides, (iii) one or more APCs comprising the one or more peptides or the polynucleotide encoding the one or more peptides, or (iv) a T cell receptor (TCR) specific for a neoepitope of the one or more peptides in complex with an HLA protein, and
(b) treating the patient with a therapeutic regimen that comprises the first therapeutic agent if the biomarker is present; or treating the patient with a therapeutic regimen that does not include the first therapeutic agent if the biomarker is absent, wherein the biomarker comprises a tumor microenvironment (TME) signature.

2. The method of claim 1, wherein the TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, an MHC class II signature or a functional Ig CDR3 signature.

3. The method of claim 1 or 2, wherein the B-cell signature comprises expression of a gene comprising CD20, CD21, CD3, CD22, CD24, CD27, CD38, CD40, CD72, CD79a, IGKC, IGHD, MZB1, MS4A1, CD138, BLK, CD19, FAM30A, FCRL2, MS4A1, PNOC, SPIB, TCL1A, TNFRSF17 or combinations thereof.

4. The method of claim 1 or 2, wherein the TLS signature indicates formation of tertiary lymphoid structures.

5. The method of claim 1 or 2, wherein the tertiary lymphoid structure represents aggregates of lymphoid cells.

6. The method of claim 1 or 2, wherein the TLS signature comprises expression of a gene comprising CCL18, CCL19, CCL21, CXCL13, LAMP3, LTB, MS4A1, or combinations thereof.

7. The method of claim 1 or 2, wherein the TIS signature comprises an inflammatory gene, a cytokine, a chemokine, a growth factor, a cell surface interaction protein, a granulation factor, or a combination thereof.

8. The method of claim 1 or 2, wherein the TIS signature comprises CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT or a combination thereof.

9. The method of claim 1 or 2, wherein the effector/memory-like CD8+ T cell signature comprises expression of a gene comprising CCR7, CD27, CD45RO, CCR7, FLT3LG, GRAP2, IL16, IL7R, LTB, S1PR1, SELL, TCF7, CD62L, or any combination thereof.

10. The method of claim 1 or 2, wherein the HLA-E/CD94 signature comprises expression of a gene CD94 (KLRD1), CD94 ligand, HLA-E, KLRC1 (NKG2A), KLRB1 (NKG2C) or any combination thereof.

11. The method of claim 1 or 2, wherein the HLA-E/CD94 signature further comprises an HLA-E:CD94 interaction level.

12. The method of claim 1 or 2, wherein the NK cell signature comprises expression of a gene CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2 or a combination thereof.

13. The method of claim 1 or 2, wherein the MHC class II signature comprises expression of a gene that is an HLA comprising HLA-DMA, HLA-DOA, HLA-DPA1, HLA-DPB1, HLA-DQB1, HLA-DRA, HLA-DRB1, HLA-DRB5 or a combination thereof.

14. The method of claim 1 or 2, wherein the biomarker comprises a subset of TME gene signature comprising a Tertiary Lymphoid Structures (TLS) signature; wherein the TLS signature comprises a gene CCL18, CCL19, CCL21, CXCL13, LAMP3, LTB, MS4A1, or combinations thereof.

15. The method of claim 1 or 2, wherein the functional Ig CDR3 signature comprises an abundance of functional Ig CDR3s.

16. The method of claim 15, wherein the abundance of functional Ig CDR3s is determined by RNA-seq.

17. The method of claim 15 or 16, wherein the abundance of functional Ig CDR3s is an abundance of functional Ig CDR3s from cells of a TME sample from a subject.

18. The method of any one of claims 15-17, wherein the abundance of functional Ig CDR3s is 2\hat{ }7 or more functional Ig CDR3s.

19. The method of any one of the claims 1-18, wherein the method further comprises: administering to the biomarker positive patient the first therapeutic agent, an altered dose or time interval of the first therapeutic agent, or a second therapeutic agent.

20. The method of any one of the claims 1-18, wherein the method further comprises: not administering to the biomarker negative patient the first therapeutic agent or a second therapeutic agent.

21. The method of any one of the claims 1-18, wherein the method further comprises administering to the biomarker positive patient, an increased dose of the first therapeutic agent.

22. The method of any one of the claims 1-18, wherein the method further comprises modifying a time interval of administration of the first therapeutic agent to the biomarker positive or negative patient.

23. A method for testing a patient having a tumor for the presence or absence of a baseline biomarker that predicts that the patient is likely to have an anti-tumor response to a treatment with a therapeutic agent comprising (i) one or more peptides comprising a neoepitope of a protein, (ii) a polynucleotide encoding the one or more peptides, (iii) one or more APCs comprising the one or more peptides or the polynucleotide encoding the one or more peptides, or (iv) a T cell receptor (TCR) specific for a neoepitope of the one or more peptides in complex with an HLA protein, the method comprising:
(a) obtaining a baseline sample that has been isolated from the tumor of the patient; measuring the baseline expression level of each gene in a tumor microenvironment (TME) gene or a subset of said genes;
(b) normalizing the measured baseline expression levels; calculating a baseline signature score for the TME gene signature from the normalized expression levels;
(c) comparing the baseline signature score to a reference score for the TME gene signature; and,
(d) classifying the patient as biomarker positive or biomarker negative for an outcome related to a durable clinical benefit (DCB) from the therapeutic agent.

24. The method of claim 23, wherein the TME signature comprises a signature of one or more of claims 2-18, or a subset thereof.

25. A pharmaceutical composition for use in treating cancer in a patient who tests positive for a biomarker, wherein the composition the therapeutic agent comprises (a) one or more peptides comprising a neoepitope of a protein, (b) a polynucleotide encoding the one or more peptides, (c) one or more APCs comprising the one or more peptides or the polynucleotide encoding the one or more peptides, or (d) a T cell receptor (TCR) specific for a neoepitope of the one or more peptides in complex with an HLA protein; and at least one pharmaceutically acceptable excipient; and wherein the biomarker is an on-treatment biomarker which comprises a gene signature selected from the group consisting of TME gene signature comprises a B-cell signature, a Tertiary Lymphoid Structures (TLS) signature, a Tumor Inflammation Signature (TIS), an effector/memory-like CD8+ T cell signature, an HLA-E/CD94 signature, a NK cell signature, and an MHC class II signature.

26. The pharmaceutical composition of claim 25, wherein the TME signature comprises a signature of any one or more of claims 2-18, or a subset thereof.

27. A method of treating cancer in a subject in need thereof, comprising: administering a therapeutically effective amount of a cancer therapeutic agent, wherein the subject has an increased likelihood of responding to the cancer therapeutic agent, wherein the subject's increased likelihood of responding to the cancer therapeutic agent is associated with the presence of one or more peripheral blood mononuclear cell signatures prior to treatment with the cancer therapeutic agent; and wherein at least one of the one or more peripheral blood mononuclear cell signatures comprises a threshold value for a ratio of cell counts of a first mononuclear cell type to a second mononuclear cell type in the peripheral blood of the subject.

28. The method of claim 27, wherein the cancer is melanoma.

29. The method of claim 27, wherein the cancer is non-small cell lung cancer.

30. The method of claim 27, wherein the cancer is bladder cancer.

31. The method of claim 27, wherein the cancer therapeutic comprises a neoantigen peptide vaccine.

32. The method of claim 27, wherein the cancer therapeutic comprises an anti-PD1 antibody.

33. The method of claim 27, wherein the cancer therapeutic comprises a combination of the neoantigen vaccine and the anti-PD1 antibody, wherein the neoantigen vaccine is administered or co-administered after a period of administering anti-PD1 antibody alone.

34. The method of claim 32 or 33, wherein the anti-PD1 antibody is nivolumab.

35. The method of claim 27, wherein the threshold value is a maximum threshold value.

36. The method of claim 27, wherein the threshold value is a minimum threshold value.

37. The method of claim 27, wherein at least one of the one or more peripheral blood mononuclear cell signatures comprises a maximum threshold value for a ratio of naïve CD8+ T cells to total CD8+ T cells in a peripheral blood sample from the subject.

38. The method of claim 37, wherein the maximum threshold value for the ratio of naïve CD8+ T cells to total CD8+ T cells in the peripheral blood sample from the subject is about 20:100.

39. The method of claim 37 or 38, wherein the peripheral blood sample from the subject has a ratio of naïve CD8+ T cells to total CD8+ T cells that is 20:100 or less or less than 20:100.

40. The method of claim 27, wherein at least one of the one or more peripheral blood mononuclear cell signatures comprises a minimum threshold value for a ratio of effector memory CD8+ T cells to total CD8+ T cells in a peripheral blood sample from the subject.

41. The method of claim 40, wherein the minimum threshold value for the ratio of effector memory CD8+ T cells to total CD8+ T cells in the peripheral blood sample from the subject is about 40:100.

42. The method of claim 40 or 41, wherein the peripheral blood sample from the subject has a ratio of effector memory CD8+ T cells to total CD8+ T cells that is 40:100 or more or more than 40:100.

43. The method of claim 27, wherein at least one of the one or more peripheral blood mononuclear cell signatures comprises a minimum threshold value for a ratio of class-switched memory B cells to total CD19+ B cells in a peripheral blood sample from the subject.

44. The method of claim 43, wherein the minimum threshold value for the ratio of class-switched memory B cells to total CD19+ B cells in the peripheral blood sample from the subject is about 10:100.

45. The method of claim 43 or 44, wherein the peripheral blood sample from the subject has a ratio of class-switched memory B cells to total CD19+ B cells that is 10:100 or more or more than 10:100.

46. The method of claim 27, wherein at least one of the one or more peripheral blood mononuclear cell signatures comprises a maximum threshold value for a ratio of naïve B cells to total CD19+ B cells in a peripheral blood sample from the subject.

47. The method of claim 46, wherein the maximum threshold value for the ratio of naïve B cells to total CD19+ B cells in the peripheral blood sample from the subject is about 70:100.

48. The method of claim 46 or 47, wherein the peripheral blood sample from the subject has a ratio of naïve B cells to total CD19+ B cells that is 70:100 or less or less than 70:100.

49. The method of any one of the claims 37-48, wherein the cancer is a melanoma.

50. The method of claim 27, wherein at least one of the one or more peripheral blood mononuclear cell signatures comprises a maximum threshold value for a ratio of plasmacytoid dendritic cells to total Lin−/CD11c− cells in a peripheral blood sample from the subject.

51. The method of claim 50, wherein the maximum threshold value for the ratio of plasmacytoid dendritic cells to total Lin−/CD11c− cells in the peripheral blood sample from the subject is about 3:100.

52. The method of claim 50 or 51, wherein the peripheral blood sample from the subject has a ratio of plasmacytoid dendritic cells to total Lin−/CD11c− cells that is 3:100 or less or less than 3:100.

53. The method of claim 27, wherein at least one of the one or more peripheral blood mononuclear cell signatures comprises a maximum threshold value for a ratio of CTLA4+ CD4 T cells to total CD4+ T cells in a peripheral blood sample from the subject.

54. The method of claim 50, wherein the maximum threshold value for the ratio of CTLA4+ CD4 T cells to total CD4+ T cells in the peripheral blood sample from the subject is about 9:100.

55. The method of claims 50 and 51, wherein the peripheral blood sample from the subject has a ratio of CTLA4+ CD4 T cells to total CD4+ T cells that is 9:100 or less or less than 9:100.

56. The method of any one of the claims 50-55, wherein the cancer is a non-small cell lung cancer.

57. The method of claim 27, wherein at least one of the one or more peripheral blood mononuclear cell signatures comprises a minimum threshold value for a ratio of memory CD8+ T cells to total CD8+ T cells in a peripheral blood sample from the subject.

58. The method of claim 57, wherein the minimum threshold value for the ratio of memory CD8+ T cells to total CD8+ T cells in the peripheral blood sample from the subject is about 40:100 or about 55:100.

59. The method of claims 57 and 58, wherein the peripheral blood sample from the subject has a ratio of memory CD8+ T cells to total CD8+ T cells that is 40:100 or more or more than 40:100.

60. The method of claims 57 and 58, wherein the peripheral blood sample from the subject has a ratio of memory CD8+ T cells to total CD8+ T cells that is 55:100 or more or more than 55:100.

61. The method of any one of the claims 57-60, wherein the cancer is a bladder cancer.

62. A method of treating cancer in a subject in need thereof, comprising: administering to the subject a therapeutically effective amount of a cancer therapeutic agent, wherein the subject has an increased likelihood of responding to the cancer therapeutic agent, and wherein the subject's increased likelihood of responding to the cancer therapeutic agent is associated with a clonal composition characteristic of TCR repertoires analyzed from peripheral blood sample of the subject at least at a timepoint prior to administering the cancer therapeutic agent.

63. The method of claim 62, wherein the clonal composition characteristic of TCR repertoires in a prospective patient is defined by a relatively low TCR diversity versus the TCR diversity in healthy donors.

64. The method of claim 62 or 63, wherein the clonal composition characteristic is analyzed by a method comprising sequencing the TCRs or fragments thereof.

65. The method of claim 62, wherein the clonal composition characteristic of TCR repertoires is defined by the clonal frequency distribution of the TCRs.

66. The method of any one of the claims 62-65, wherein the clonal composition characteristic of the TCR repertoires is further analyzed by calculating the frequency distribution pattern of TCR clones.

67. The method of claim 66, wherein the frequency distribution pattern of TCR clones is analyzed using one or more of: Gini Coefficient, Shannon entropy, DE50, Sum of Squares, and Lorenz curve.

68. The method of claim 62, wherein the subject's increased likelihood of responding to the cancer therapeutic agent is associated with increased clonality of the TCRs.

69. The method of claim 62, wherein the subject's increased likelihood of responding to the cancer therapeutic agent is associated with increased frequency of medium and/or large and/or hyperexpanded sized TCR clones.

70. The method of claim 62, wherein the subject's increased likelihood of responding to the cancer therapeutic agent is associated with a clonal composition characteristic of TCR repertoires according to any one of claims 63-69, wherein the clonal composition characteristic is analyzed from peripheral blood sample of the subject prior to administering a therapeutically effective amount of a cancer therapeutic agent.

71. The method of claim 62, wherein a clonal composition characteristic of TCR repertoires comprises a measure of the clonal stability of the TCRs.

72. The method of claim 70 or 71, wherein the clonal stability of the TCRs is analyzed as TCR turnover between a first and a second timepoints, wherein the first timepoint is prior to administering the cancer therapeutic agent and the second timepoint is a timepoint during the duration of the treatment.

73. The method of claim 71, wherein the second timepoint is prior to administering the vaccine.

74. The method of claim 70, wherein the clonal stability of TCRs is analyzed using a Jensen-Shannon Divergence.

75. The method of claim 70, wherein the subject's increased likelihood of responding to the cancer therapeutic agent is associated with higher TCR stability.

76. The method of claim 70, wherein the subject's increased likelihood of responding to the cancer therapeutic agent is associated with reduced turnover of T cell clones between the first timepoint and the second timepoint.

77. A method of treating cancer in a subject in need thereof, comprising: administering a therapeutically effective amount of a cancer therapeutic agent to the subject, wherein the subject has an increased likelihood of responding to the cancer therapeutic agent, wherein the subject's increased likelihood of responding to the cancer therapeutic agent is associated with the presence of one or more genetic variations in the subject, wherein the subject has been tested for a presence of the one or more genetic variations with an assay and has been identified as having the one or more genetic variations, wherein the one or more genetic variations comprise an ApoE allele genetic variation comprising (i) an ApoE2 allele genetic variation comprising a sequence encoding a R158C ApoE protein or (ii) an ApoE4 allele genetic variation comprising a sequence encoding a C112R ApoE protein.

78. The method of claim 77, wherein the cancer therapeutic agent comprises a neoantigen peptide vaccine.

79. The method of claim 77, wherein the cancer therapeutic agent further comprises an anti-PD1 antibody.

80. The method of claim 77, wherein the cancer therapeutic agent does not comprise an anti-PD1 antibody monotherapy.

81. The method of claim 77, wherein the cancer is melanoma.

82. The method of claim 77, wherein the subject is homozygous for the ApoE2 allele genetic variation.

83. The method of claim 77, wherein the subject is heterozygous for the ApoE2 allele genetic variation.

84. The method of claim 77, wherein the subject is homozygous for the ApoE4 allele genetic variation.

85. The method of claim 77, wherein the subject is heterozygous for the ApoE4 allele genetic variation.

86. The method of claim 77, wherein the subject comprises an ApoE allele comprising a sequence encoding a ApoE protein that is not a R158C ApoE protein or a C112R ApoE protein.

87. The method of claim 77, wherein the subject has rs7412-T and rs449358-T.

88. The method of claim 77, wherein the subject has rs7412-C and rs449358-C.

89. The method of claim 77, wherein a reference subject that is homozygous for the ApoE3 allele has a decreased likelihood of responding to the cancer therapeutic agent.

90. The method of claim 77, wherein the assay is a genetic assay.

91. The method of claim 77, wherein the cancer therapeutic agent comprises one or more peptides comprising a cancer epitope.

92. The method of claim 77, wherein the cancer therapeutic agent comprises (i) a polynucleotide encoding the one or more peptides of claim 91,
a. or, (ii) one or more APCs comprising the one or more peptides or the polynucleotide encoding the one or more peptides,
b. or (iii) a T cell receptor (TCR) specific for a cancer epitope of the one or more peptides in complex with an HLA protein.

93. The method of any one of the claims 77-92, wherein the cancer therapeutic agent further comprises an immunomodulatory agent.

94. The method of claim 93, wherein the immunotherapeutic agent is an anti-PD1 antibody.

95. The method of claim 77, wherein the cancer therapeutic agent is not nivolumab alone or pembrolizumab alone.

96. The method of claim 77, wherein the one or more genetic variations comprises chr19:44908684 T>C; wherein chromosome positions of the one or more genetic variations are defined with respect to UCSC hg38.

97. The method of claim 77, wherein the one or more genetic variations comprises chr19:44908822 C>T; wherein chromosome positions of the one or more genetic variations are defined with respect to UCSC hg38.

98. The method of claim 77, wherein the method further comprises testing the subject for the presence of the one or more genetic variations with the assay prior to the administering.

99. The method of claim 77, wherein the ApoE2 allele genetic variation is a germline variation.

100. The method of claim 77, wherein the ApoE4 allele genetic variation is a germline variation.

101. The method of claim 77, wherein the method comprises administering to the subject a cancer therapeutic agent comprising one or more peptides comprising a cancer epitope; wherein the subject is determined as having the germline ApoE4 allelic variant.

102. The method of claim 101, wherein the therapeutic agent further comprises one or more of: an adjuvant therapy, a cytokine therapy, or an immunomodulator therapy.

103. The method of claim 101 or 102, wherein the immunomodulator therapy is a PD1 inhibitor, such as an anti-PD1 antibody.

104. The method of any one of the claims 101-103, wherein the therapeutic agent does not comprise a PD1 inhibitor monotherapy.

105. The method of claim 77, wherein the method further comprises administering an agent that promotes ApoE activity or comprises ApoE activity.

106. The method of claim 77, wherein the method further comprises administering an agent that inhibits ApoE activity.

107. The method of any one of the preceding claims, where the cancer is a pancreatic cell cancer.

108. The method of any one of the preceding claims, wherein the therapeutic agent comprises a vaccine.

109. The method of any one of the preceding claims, wherein the therapeutic agent comprises a peptide vaccine, comprising at least one, two, three or four antigenic peptides.

110. The method of any one of the preceding claims, wherein the therapeutic agent comprises a peptide vaccine, comprising at least one, two, three or four neoantigenic peptides.

111. The method of any one of the preceding claims, wherein the therapeutic agent comprises a nucleic acid encoding a peptide, wherein the peptide is a neoantigen peptide.

112. The method of any one of the preceding claims, wherein the therapeutic agent comprises a combination therapy comprising one or more checkpoint inhibitor antibodies, and a vaccine comprising a neoantigen peptide, or a nucleic acid encoding the neoantigenic peptide.

113. The method of claim 70, wherein the clonal composition characteristic is analyzed from peripheral blood sample of the subject prior to administering a vaccine, wherein the vaccine comprises at least one peptide or a polynucleotide encoding a peptide, wherein the cancer therapeutic agent comprises a combination of a neoantigen vaccine and an anti-PD1 antibody, wherein the neoantigen vaccine is administered or co-administered after a period of administering anti-PD1 antibody alone.
